(19) United States
(12) Patent Application Publication
(10) Pub. No.: US 2010/0323442 A1
(43) Pub. Date: Dec. 23, 2010

(54) MODULATION OF THE PHOSPHATIDYLINOSITOL-3-KINASE PATHWAY IN THE DIFFERENTIATION OF HUMAN EMBRYONIC STEM CELLS

(76) Inventors: Emmanuel Edward Baetge, Encinitas, CA (US); Kevin D'Amour, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/446,149

(22) PCT Filed: Oct. 17, 2007
(86) PCT No.: PCT/US07/22182

§ 371 Date: Apr. 17, 2009

Related U.S. Application Data
(60) Provisional application No. 60/829,853, filed on Oct. 17, 2006.

(51) Int. Cl. C12N 5/071

(52) U.S. Cl. 435/375

(57) ABSTRACT

Disclosed herein are cell cultures comprising differentiated human cells and methods of producing the same. The present invention provides compositions and methods for the production of differentiated human cells from human pluripotent cells. Preferably, the differentiated cells are selected from the group consisting of mesendoderm cells, definitive endoderm cells, ectoderm cells, trophectoderm cells, and extraembryonic endoderm cells.

### FIELD OF THE INVENTION

The present invention relates to the fields of medicine and cell biology. In particular, the present invention relates to compositions and methods for differentiating and culturing pluripotent stem cells, the cells created by these methods and the uses thereof.

### BACKGROUND

Human pluripotent stem cells, such as embryonic stem (ES) cells and embryonic germ (EG) cells, were first isolated in culture without fibroblast feeders in 1994 (Bongso et al., 1994) and with fibroblast feeders (Hogan, 1997). Later, Thomson, Reubinoff and Shamblott established continuous cultures of human ES and EG cells using mitotically inactivated mouse feeder layers (Reubinoff et al., 2000; Shamblott et al., 1998; Thomson et al., 1998).

Human ES and EG cells (hESCs) offer unique opportunities for investigating early stages of human development as well as for therapeutic intervention in several disease states. Two properties that make hESCs uniquely suited to cell therapy applications are pluripotency and the ability to maintain these cells in culture for prolonged periods without accumulation of genetic changes. Pluripotency is defined by the ability of hESCs to differentiate to derivatives of all 3 primary germ layers (endoderm, mesoderm, ectoderm) which, in turn, form all cell somatic types of the mature organism in addition to extraembryonic tissues (e.g. placenta) and germ cells. Each primary germ layer has the potential to produce a readily-available supply of mature cell types which can be used in numerous therapeutic applications. For example, the use of insulin-producing β-cells derived from hESCs would offer a vast improvement over current cell therapy procedures which utilize cells from donor pancreases. As another example, cell therapy with neurons derived from hESCs would greatly benefit patients suffering from a variety of neurological disorders, such as amyotrophic lateral sclerosis, Alzheimer's disease and Parkinson's disease. Non-neural ectoderm-derived skin cells would be useful in the treatment of skin disorders. Also, a readily-available supply of cells of any of the primary lineages and tissues derived therefrom is would be extremely useful for the screening and development of therapeutic molecules as well as for in vitro toxicity screening assays. However, for any of the above-mentioned therapeutic or screening applications, substantial quantities of high quality differentiated cells are needed.

Although pluripotency imparts extraordinary utility upon hESCs, this property also poses unique challenges for the study and manipulation of these cells and their derivatives. Each of the mature cell types found in the human body pass through multiple differentiation steps along the path to maturity. At each step, multiple cell fates are possible, and as a result, the vast majority of cell types are produced at very low efficiencies. Accordingly, if differentiation is not directed along a particular path during early stages of differentiation, the end yield of any desired cell type will be low. Thus, achieving efficient, directed differentiation of the primary descendants of hESCs as well as other early precursor cells is of great importance for both therapeutic and industrial applications of cell products derived from hESCs.

In order to use hESCs as a starting material to generate cells that are useful in medical and industrial applications, such as cell therapy and cell screening, it would be advantageous to increase the initial production yield of cells of the desired primary cell lineage. As such, it would be advantageous to efficiently direct hESCs toward the desired primary cell lineage at the very earliest stages of differentiation.

### SUMMARY OF THE INVENTION

Some embodiments of the present invention relate to methods of differentiating human pluripotent cells. In such embodiments, the method comprises the steps of obtaining a population of human pluripotent cells; providing the population of human pluripotent cells with at least one differentiation factor in an amount sufficient to promote differentiation of said human pluripotent cells to cells of a cell lineage selected from the group consisting of ectoderm, trophectoderm and extraembryonic endoderm; contacting the population of human pluripotent cells with a culture medium that limits phosphotidylinositol-3-kinase (PI-3-kinase) signaling; and incubating the population of human embryonic stem cells in the culture medium for a sufficient time to allow the pluripotent cells to differentiate into cells of a cell lineage selected from the group consisting of ectoderm, trophectoderm and extraembryonic endoderm. In certain embodiments, the step of providing said human pluripotent cells with said differentiation factor occurs at about the same time or subsequent to the step of contacting said human pluripotent cells with said culture medium.

In some embodiments, the differentiation factor can be follistatin, noggin, bone morphogenic proteins (BMPs), SU5402 and combinations thereof. In certain embodiments, the culture medium includes less than about 10% serum and lacks serum replacement. In certain embodiments, the culture medium includes less than about 2 μg/ml insulin or insulin analogs, less than about 10 ng/ml of an insulin-like growth factor or insulin-like growth factor analogs and less than about 2 μg/ml of insulin-mimetic compounds. In such embodiments, the insulin-like growth factor can be insulin-like growth factor-1 (IGF-1) or insulin-like growth factor-2 (IGF-2). Further, the insulin-mimetic compounds can be, for example, vanadium(IV) oxo-bis(maltolato) (BMOV), ZnCl₂, bis(maltolato)zinc(II), zinc(II) complexes and vanadyl(IV) complexes.

In certain embodiments, the trophectoderm cells express a marker selected from the group consisting of CDX2, HAND1, Eomes, MASH2, ESXL1, HCG, KRT18, PSG3, SFXN5, DLX3, PSX1, ETS2 and ERRB. In some embodiments, the trophectoderm cells do not substantially express SOX17 or CXCR4.

In certain embodiments, the extraembryonic endoderm cells express a marker selected from the group consisting of SOX7, alpha-fetoprotein (AFP), SPARC and Thrombomodulin (TM). In some embodiments, the extraembryonic endoderm cells do not substantially express CXCR4.

In certain embodiments, the ectoderm cells express a marker selected from the group consisting of PAX6, SOX1 and ZIC1, cytokeratin, FGF5, HOXB1, LHX5, MASH1, MEIS1 and OTX1. In some embodiments, the ectoderm cells do not substantially express SOX17.

In still further embodiments, the human pluripotent cells are human embryonic stem cells (hESCs), and can be derived from, for example, the morula, the inner cell mass (ICM) of an embryo and the gonadal ridges of an embryo. In some embodiments, hESCs are derived from a preimplantation embryo.

Other embodiments relate to cell cultures comprising human cells, wherein at least 10% of said human cells are multipotent extraembryonic endoderm cells that can differentiate into cells of the visceral endoderm or parietal endoderm. In such embodiments, the amount of extraembryonic endoderm cells can range from about 10% to about 95% of the cells present in the cell culture.

Still other embodiments relate to cell cultures comprising human cells, wherein at least 10% of said human cells are multipotent trophectoderm cells that can differentiate into cells of the mural or polar trophoblast. In such embodiments, the amount of trophectoderm cells can range from about 10% to about 95% of the cells present in the cell culture.

Still further embodiments relate to cell cultures comprising human cells, wherein at least 10% of said human cells are multipotent ectoderm cells that can differentiate into cells of neural ectoderm or non-neural ectoderm. In such embodiments, the amount of ectoderm cells can range from about 10% to about 95% of the cells present in the cell culture.

Such embodiments may further comprise a medium which comprises less than about 10% serum and lacks serum replacement. In some embodiments the medium lacks serum. Such embodiments may further comprise a medium which comprises less than about 2 μg/ml insulin or insulin analogs, less than about 10 ng/ml of an insulin-like growth factor or insulin-like growth factor analogs and less than about 2 μg/ml of insulin-mimetic compounds. The insulin-like growth factor can be, for example insulin-like growth factor-1 (IGF-1) or insulin-like growth factor-1 (IGF-2), and the insulin-mimetic compounds can be, for example, vanadium(IV) oxo-bis(maltolato) (BMOV), ZnCl₂, bis(maltolato)zinc(II), zinc(II) complexes and vanadyl(IV) complexes. Such embodiments may further comprise at least one differentiation factor selected from the group consisting of follistatin, noggin, BMP, SU5402 and combinations thereof.

Other embodiments of the present inventions are described with reference to the numbered paragraphs below:

1. A method of differentiating human pluripotent cells, said method comprising the steps of obtaining a population of human pluripotent cells, providing said population of human pluripotent cells with at least one differentiation factor in an amount sufficient to promote differentiation of said human pluripotent cells to cells of a cell lineage selected from the group consisting of ectoderm, trophectoderm and extraembryonic endoderm, contacting said population of human pluripotent cells with a culture medium that decreases or limits phosphotidylinositol-3-kinase (PI-3-kinase) signaling, and incubating said population of human pluripotent cells in said culture medium for a sufficient time to allow said pluripotent cells to differentiate into cells of a cell lineage selected from the group consisting of ectoderm, trophectoderm and extraembryonic endoderm.

2. The method of Paragraph 1, wherein said at least about 10% of the human pluripotent cells differentiate into cells of a cell lineage selected from the group consisting of trophectoderm, ectoderm and extraembryonic endoderm.

3. The method of Paragraph 1, wherein said at least about 50% of the human pluripotent cells differentiate into cells of a cell lineage selected from the group consisting of trophectoderm, ectoderm and extraembryonic endoderm.

4. The method of Paragraph 1, wherein said at least about 80% of the human pluripotent cells differentiate into cells of a cell lineage selected from the group consisting of trophectoderm, ectoderm and extraembryonic endoderm.

5. The method of Paragraph 1, wherein said at least one differentiation factor is selected from the group consisting of follistatin, noggin, bone morphogenetic protein (BMP), a fibroblast growth factor receptor (FGFR) inhibitor and combinations thereof.

6. The method of Paragraph 5, wherein said at least one differentiation factor is provided in said culture medium at a concentration ranging from about 1 ng/ml to about 1000 ng/ml.

7. The method of Paragraph 1, wherein said culture medium comprises less than about 10% serum and lacks serum replacement.

8. The method of Paragraph 7, wherein said culture medium comprises less than about 5% serum.

9. The method of Paragraph 7, wherein said culture medium comprises less than about 2% serum.

10. The method of Paragraph 7, wherein said culture medium comprises less than about 1% serum.

11. The method of Paragraph 7, wherein said culture medium comprises less than about 0.5% serum.

12. The method of Paragraph 7, wherein said culture medium comprises less than about 0.2% serum.

13. The method of Paragraph 7, wherein said culture medium lacks serum.

14. The method of Paragraph 13, wherein after about 1 day from initiation of said contacting step about 0.2% serum is present in the culture medium.

15. The method of Paragraph 13, wherein after about 2 days from initiation of said contacting step from about 0.2% to about 2% serum is present in the culture medium.

16. The method of Paragraph 1, wherein said culture medium comprises less than about 2 μg/ml insulin or insulin analogs, less than about 10 ng/ml of an insulin-like growth factor or insulin-like growth factor analogs and less than about 2 μg/ml of insulin-mimetic compounds.

17. The method of Paragraph 1, wherein said culture medium comprises less than about 1 μg/ml insulin or insulin analogs, less than about 5 ng/ml of an insulin-like growth factor or insulin-like growth factor analogs and less than about 1 μg/ml of insulin-mimetic compounds.

18. The method of Paragraph 1, wherein said culture medium comprises less than about 500 ng/ml insulin or insulin analogs, less than about 2 ng/ml of an insulin-like growth factor or insulin-like growth factor analogs and less than about 500 ng/ml of insulin-mimetic compounds.

19. The method of Paragraph 1, wherein said culture medium comprises less than about 100 ng/ml insulin or insulin analogs, less than about 1 ng/ml of an insulin-like growth factor or insulin-like growth factor analogs and less than about 100 ng/ml of insulin-mimetic compounds.

20. The method of Paragraph 1, wherein said culture medium lacks a substantial concentration of a molecule selected from the group consisting of insulin, insulin analogs, insulin-like growth factors, insulin-like growth factor analogs, insulin-mimetic compounds and combinations thereof.

21. The method of Paragraph 1, wherein said culture medium lacks a substantial concentration of insulin, insulin analogs, insulin-like growth factors, insulin-like growth factor analogs, insulin-mimetic compounds and combinations thereof.

22. The method of any one of Paragraphs 16, 17, 18, 19, 20 or 21, wherein said insulin-like growth factor is insulin-like growth factor-1 (IGF-1).

23. The method of any one of Paragraphs 16, 17, 18, 19, 20 or 21, wherein said insulin-like growth factor is insulin-like growth factor-2 (IGF-2).

24. The method of any one of Paragraphs 16, 17, 18, 19, 20 or 21, wherein said insulin-mimetic compounds are selected from the group consisting of vanadium(IV) oxo-bis(maltolato) (BMOV), ZnCl₂, bis(maltolato)zinc(II), zinc(II) complexes and vanadyl(IV) complexes.

25. The method of Paragraph 1, wherein said trophectoderm cells express a marker selected from the group consisting of CDX2, HAND1, Eomes, MASH2, ESXL1, HCG, KRT18, PSG3, SFXN5, DLX3, PSX1, ETS2 and ERRB.

26. The method of Paragraph 25, wherein said trophectoderm cells express CDX2.

27. The method of Paragraph 26, wherein said trophectoderm cells do not substantially express SOX17 or CXCR4.

28. The method of Paragraph 1, wherein said extraembryonic endoderm cells express a marker selected from the group consisting of SOX7, alpha-fetoprotein (AFP), SPARC and Thrombomodulin (TM).

29. The method of Paragraph 28, wherein said extraembryonic endoderm cells express SOX7.

30. The method of Paragraph 29, wherein said extraembryonic endoderm cells do not substantially express CXCR4.

31. The method of Paragraph 1, wherein said ectoderm cells express a marker selected from the group consisting of PAX6, SOX1 and ZIC1, cytokeratin, FGF5, HOXB1, LHX5, MASH1, MEIS1 and OTX1.

32. The method of Paragraph 31, wherein said ectoderm cells express PAX6 and SOX1.

33. The method of Paragraph 32, wherein said ectoderm cells do not substantially express SOX17.

34. The method of Paragraph 1, wherein said human pluripotent cells are human embryonic stem cells (hESCs).

35. The method of Paragraph 34, wherein said hESCs are derived from a tissue selected from the group consisting of the morula, the inner cell mass (ICM) of an embryo and the gonadal ridges of an embryo.

36. The method of Paragraph 1, wherein said step of providing said human pluripotent cells with said differentiation factor occurs at about the same time or subsequent to the step of contacting said human pluripotent cells with said culture medium.

37. A cell culture comprising human cells, wherein at least 10% of said human cells are trophectoderm cells, said trophectoderm cells being multipotent cells that can differentiate into cells of the mural or polar trophoblast.

38. The cell culture of Paragraph 37, wherein at least about 50% of said human cells are trophectoderm cells.

39. The cell culture of Paragraph 37, wherein at least about 80% of said human cells are trophectoderm cells.

40. The cell culture of Paragraph 37, wherein at least about 90% of said human cells are trophectoderm cells.

41. The cell culture of Paragraph 37, wherein at least about 95% of said human cells are trophectoderm cells.

42. The cell culture of Paragraph 37, wherein human feeder cells are present in said culture, and wherein at least about 10% of human cells other than said human feeder cells are trophectoderm cells.

43. The cell culture of Paragraph 37, wherein human feeder cells are present in said culture, and wherein at least about 50% of human cells other than said human feeder cells are trophectoderm cells.

44. The cell culture of Paragraph 37, wherein said trophectoderm cells express a marker selected from the group consisting of CDX2, HAND1, Eomes, MASH2, ESXL1, HCG, KRT18, PSG3, SFXN5, DLX3, PSX1, ETS2, and ERRB.

45. The cell culture of Paragraph 44, wherein said trophectoderm cells express CDX2.

46. The cell culture of Paragraph 45, wherein said trophectoderm do not substantially express SOX17 or CXCR4.

47. The cell culture of Paragraph 37, wherein said cell culture further comprises human pluripotent cells.

48. The cell culture of Paragraph 47, wherein at least about 2 trophectoderm cells are present for about every 1 pluripotent cell in said cell culture.

49. A cell culture comprising human cells, wherein at least 10% of said human cells are extraembryonic endoderm cells, said extraembryonic endoderm cells being multipotent cells that can differentiate into cells of the visceral endoderm or parietal endoderm.

50. The cell culture of Paragraph 49, wherein at least about 50% of said human cells are extraembryonic endoderm cells.

51. The cell culture of Paragraph 49, wherein at least about 80% of said human cells are extraembryonic endoderm cells.

52. The cell culture of Paragraph 49, wherein at least about 90% of said human cells are extraembryonic endoderm cells.

53. The cell culture of Paragraph 49, wherein at least about 95% of said human cells are extraembryonic endoderm cells.

54. The cell culture of Paragraph 49, wherein human feeder cells are present in said culture, and wherein at least about 10% of human cells other than said human feeder cells are extraembryonic endoderm cells.

55. The cell culture of Paragraph 49, wherein human feeder cells are present in said culture, and wherein at least about 50% of human cells other than said human feeder cells are extraembryonic endoderm cells.

56. The cell culture of Paragraph 49, wherein said extraembryonic endoderm cells express a marker selected from the group consisting of SOX7, alpha-fetoprotein (AFP), SPARC and Thrombomodulin (TM).

57. The cell culture of Paragraph 56, wherein said extraembryonic endoderm cells express SOX7.

58. The cell culture of Paragraph 57, wherein said extraembryonic endoderm cells do not substantially express CXCR4.

59. The cell culture of Paragraph 49, wherein said cell culture further comprises human pluripotent cells.

60. The cell culture of Paragraph 59, wherein at least about 2 extraembryonic endoderm cells are present for about every 1 pluripotent cell in said cell culture.

61. A cell culture comprising human cells, wherein at least 10% of said human cells are ectoderm cells, said ectoderm cells being multipotent cells that can differentiate into cells of neural ectoderm or non-neural ectoderm.

62. The cell culture of Paragraph 61, wherein at least about 50% of said human cells are ectoderm cells.

63. The cell culture of Paragraph 61, wherein at least about 80% of said human cells are ectoderm cells.

64. The cell culture of Paragraph 61, wherein at least about 90% of said human cells are ectoderm cells.

65. The cell culture of Paragraph 61, wherein at least about 95% of said human cells are ectoderm cells.

66. The cell culture of Paragraph 61, wherein human feeder cells are present in said culture, and wherein at least about 10% of human cells other than said human feeder cells are ectoderm cells.

67. The cell culture of Paragraph 61, wherein human feeder cells are present in said culture, and wherein at least about 50% of human cells other than said human feeder cells are ectoderm cells.

68. The cell culture of Paragraph 61, wherein said ectoderm cells express a marker selected from the group consisting of PAX6, SOX1 and ZIC1, cytokeratin, FGF5, HOXB1, LHX5, MASH1, MEIS1 and OTX1.

69. The cell culture of Paragraph 68, wherein said ectoderm cells express a marker selected from the group consisting of PAX6, SOX1 and ZIC1.

70. The cell culture of Paragraph 68, wherein said ectoderm cells express PAX6, SOX1 and ZIC1.

71. The cell culture of Paragraph 69 or Paragraph 70, wherein said ectoderm cells do not substantially express SOX17.

72. The cell culture of Paragraph 61, wherein said cell culture further comprises human pluripotent cells.

73. The cell culture of Paragraph 72, wherein at least about 2 ectoderm cells are present for about every 1 pluripotent cell in said cell culture.

74. The cell culture of any one of Paragraphs 37, 49 or 61, wherein said human cells comprises embryonic stem cells.

75. The cell culture of Paragraph 74, wherein said embryonic stem cells are derived from a tissue selected from the group consisting of the morula, the inner cell mass (ICM) of an embryo and the gonadal ridges of an embryo.

76. The cell culture of any of Paragraphs 37, 49 or 61 further comprising a medium which comprises less than about 10% serum and lacks serum replacement.

77. The cell culture of Paragraph 76, wherein said medium comprises less than about 5% serum.

78. The cell culture of Paragraph 76, wherein said medium comprises less than about 2% serum.

79. The cell culture of Paragraph 76, wherein said medium comprises less than about 1% serum.

80. The cell culture of Paragraph 76, wherein said medium comprises less than about 0.5% serum.

81. The cell culture of Paragraph 76, wherein said medium comprises less than about 0.2% serum.

82. The cell culture of Paragraph 76, wherein said medium lacks serum.

83. The cell culture of any one of Paragraphs 37, 49 or 61 further comprising a medium which comprises less than about 2 μg/ml insulin or insulin analogs, less than about 10 ng/ml of an insulin-like growth factor or insulin-like growth factor analogs and less than about 2 μg/ml of insulin-mimetic compounds.

84. The cell culture of Paragraph 83 further comprising a medium which comprises less than about 1 μg/ml insulin or insulin analogs, less than about 5 ng/ml of an insulin-like growth factor or insulin-like growth factor analogs and less than about 1 μg/ml of insulin-mimetic compounds.

85. The cell culture of any of Paragraphs 83 further comprising a medium which comprises less than about 500 ng/ml insulin or insulin analogs, less than about 2 ng/ml of an insulin-like growth factor or insulin-like growth factor analogs and less than about 500 ng/ml of insulin-mimetic compounds.

86. The cell culture of any of Paragraphs 83 further comprising a medium which comprises less than about 100 ng/ml insulin or insulin analogs, less than about 1 ng/ml of an insulin-like growth factor or insulin-like growth factor analogs and less than about 100 ng/ml of insulin-mimetic compounds.

87. The cell culture of Paragraph 83, wherein said culture medium lacks a substantial concentration of a molecule selected from the group consisting of insulin, insulin analogs, insulin-like growth factors, insulin-like growth factor analogs, insulin-mimetic compounds and combinations thereof.

88. The method of Paragraph 83, wherein said culture medium lacks a substantial concentration of insulin, insulin analogs, insulin-like growth factors, insulin-like growth factor analogs, insulin-mimetic compounds and combinations thereof.

89. The cell culture of Paragraph 83, wherein said insulin-like growth factor is insulin-like growth factor-1 (IGF-1).

90. The cell culture of Paragraph 83, wherein said insulin-like growth factor is insulin-like growth factor-1 (IGF-2).

91. The cell culture of Paragraph 83, wherein said insulin-mimetic compounds are selected from the group consisting of: vanadium(IV) oxo-bis(maltolato) (BMOV), ZnCl₂, bis(maltolato)zinc(II), zinc(II) complexes and vanadyl(IV) complexes.

92. The cell culture of any of Paragraphs 37, 49 or 61 further comprising at least one differentiation factor selected from the group consisting of follistatin, noggin, BMP, a fibroblast growth factor receptor (FGFR) inhibitor and combinations thereof.

93. The cell culture of Paragraph 92, wherein said at least one differentiation factor is present in said culture medium at a concentration ranging from about 1 ng/ml to about 1000 ng/ml.

94. The cell culture of Paragraph 92, wherein said FGFR inhibitor is present in said culture medium at a concentration ranging from about 0.05 μM to about 50 μm.

95. The cell culture of Paragraph 94, wherein said FGFR inhibitor is SU5402.

96. The cell culture of any one of Paragraphs 37, 49 or 61, wherein said human cells comprises embryonic stem cells that are derived from a preimplantation embryo.

97. A method of identifying a differentiation factor capable of promoting the differentiation of a human cell type in a cell population comprising human cells, said method comprising the steps of obtaining a cell population comprising a human cell type, providing a candidate differentiation factor to said cell population, determining expression of a marker in said cell population at a first time point, determining expression of the same marker in said cell population at a second time point, wherein said second time point is subsequent to said first time point and wherein said second time point is subsequent to providing said cell population with said candidate differentiation factor, and determining if expression of the marker in said cell population at said second time point is increased or decreased as compared to the expression of the marker in said cell population at said first time point, wherein an increase or decrease in expression of said marker in said cell population indicates that said candidate differentiation factor is capable of promoting the differentiation of said human cell type, and wherein said human cell type is selected from the group consisting of trophectoderm cells, extraembryonic endoderm cells and ectoderm cells.

98. The method of Paragraph 97, wherein the cells of said human cell type differentiate into a cell selected from the group consisting of a neural ectoderm cell, a non-neural ectoderm cell, a neuron, a visceral endoderm cell, a parietal endoderm cell, a mural trophoblast cell or a polar trophoblast cell, in response to said candidate differentiation factor.

99. The method of Paragraph 97, wherein the cells of said human cell type differentiate into a precursor cell selected from the group consisting of a precursor of a neural ectoderm cell, a precursor of a non-neural ectoderm cell, a precursor of a neuron, a precursor of a visceral endoderm cell, a precursor of a parietal endoderm cell, a precursor of a mural trophoblast cell or a precursor of a polar trophoblast cell, in response to said candidate differentiation factor.

100. The method of Paragraph 97, wherein the cells of said human cell type are contacted with a culture medium that limits phosphotidylinositol-3-kinase (PI-3-kinase) signaling.

101. The method of Paragraph 97, wherein said culture medium comprises an effective amount of a phosphatidylinositol-3-kinase (PI-3-K) pathway inhibitor.

102. The method of Paragraph 101, wherein said PI-3-K pathway inhibitor is selected from the group consisting of rapamycin, LY 294002, wortmannin, lithium chloride, Akt inhibitor 1, Akt inhibitor II (SH-5), Akt inhibitor III (SH-6), NL-71-101 and combinations thereof.

103. The method of Paragraph 97, wherein said culture medium lacks a substantial concentration or an effective amount of a PI-3-K pathway activator.

104. The method of Paragraph 103, wherein said PI-3-K pathway activator is selected from the group consisting of serum, insulin, insulin analogs, insulin-like growth factors, insulin-like growth factor analogs, insulin mimetics and combinations thereof.

105. The method of Paragraph 1, wherein said culture medium comprises an effective amount of a phosphatidylinositol-3-kinase (PI-3-K) pathway inhibitor.

106. The method of Paragraph 105, wherein said PI-3-K pathway inhibitor is selected from the group consisting of rapamycin, LY 294002, wortmannin, lithium chloride, Akt inhibitor 1, Akt inhibitor II (SH-5), Akt inhibitor III (SH-6), NL-71-101 and combinations thereof.

107. The method of Paragraph 105, wherein said effective amount of said PI-3-K inhibitor ranges from about 0.1 nM to about 500 μM.

108. The method of Paragraph 105, wherein said effective amount of said PI-3-K inhibitor ranges from about 1 nM to about 100 μM.

109. The method of Paragraph 105, wherein said effective amount of said PI-3-K inhibitor ranges from about 10 nM to about 10 μM.

110. The method of Paragraph 105, wherein said effective amount of said PI-3-K inhibitor, ranges from about 100 nM to about 1 μM.

111. The method of Paragraph 1, wherein sufficient time to allow the pluripotent cells to differentiate into cells of a cell lineage is determined by detecting the presence of the cell lineage in the population of human pluripotent cells, wherein detecting the presence of the cell lineage in the cell population comprises detecting the expression of at least one marker selected from the group consisting of PAX6, SOX1, SOX7 and CDX2.

112. The method of Paragraph 111, wherein expression of said at least one marker is determined by quantitative polymerase chain reaction (Q-PCR).

113. The method of Paragraph 111, wherein expression of said at least one marker is determined by immunocytochemistry.

114. The method of Paragraph 5, wherein said FGFR inhibitor is SU5402.

115. The method of Paragraph 34, wherein said hESC is derived from a preimplantation embryo.

116. A method of differentiating human pluripotent cells, said method comprising the steps of obtaining a population of human pluripotent cells, providing said population of human pluripotent cells with at least one differentiation factor in an amount sufficient to promote differentiation of said human pluripotent cells to cells of a cell lineage selected from the group consisting of mesendoderm and definitive endoderm, contacting said population of human pluripotent cells with a culture medium that decreases or limits phosphotidylinositol-3-kinase (PI-3-kinase) signaling, and incubating said population of human pluripotent cells in said culture medium for a sufficient time to allow said pluripotent cells to differentiate into cells of a cell lineage selected from the group consisting of ectoderm, trophectoderm and extraembryonic endoderm.

117. The method of Paragraph 116, wherein the differentiation factor is selected from the group consisting of Activin A, noggin, follistatin, bone morphogenetic protein (BMP) and a fibroblast growth factor receptor (FGFR) inhibitor and a combination thereof.

118. The method of claim 117, wherein the differentiation factor is a combination of follistatin and noggin.

119. The method of Paragraph 117, wherein the differentiation factor is Activin A.

120. The method of Paragraph 117, wherein the FGFR inhibitor is SU5402.

121. The method of Paragraph 116, wherein sufficient time to allow the pluripotent cells to differentiate into cells of a cell lineage is determined by detecting the presence of the cell lineage in the population of human pluripotent cells, wherein detecting the presence of the cell lineage in the cell population comprises detecting the expression of at least one marker selected from the group consisting of Brachyury, Wnt3, SOX17 and GSC.

122. The method of Paragraph 121, wherein detecting the presence of mesendoderm cells in the population of human pluripotent cells comprises detecting the expression of at least Brachyury or Wnt3.

123. The method of Paragraph 121, wherein detecting the presence of definitive endoderm in the population of human pluripotent cells comprises detecting the expression of at least SOX17 and GSC.

124. The method of Paragraph 121, wherein the expression of at least one of said markers is determined by Q-PCR.

125. The method of Paragraph 121, wherein the expression of at least one of said markers is determined by immunocytochemistry.

It will be appreciated that the methods and compositions described above relate to cells cultured in vitro. However, the above-described in vitro differentiated cell compositions may be used for in vivo applications.

Additional embodiments of the present invention may also be found in U.S. Provisional Patent Application No. 60/532,004, entitled DEFINITIVE ENDODERM, filed Dec. 23, 2003; U.S. Provisional Patent Application No. 60/586,566, entitled CHEMOKINE CELL SURFACE RECEPTOR FOR THE ISOLATION OF DEFINITIVE ENDODERM, filed Jul. 9, 2004; U.S. Provisional Patent Application No. 60/587,942, entitled CHEMOKINE CELL SURFACE RECEPTOR FOR THE ISOLATION OF DEFINITIVE ENDODERM, filed Jul. 14, 2004; U.S. patent application Ser. No. 11/021,618, entitled DEFINITIVE ENDODERM, filed Dec. 23, 2004; U.S. patent application Ser. No. 11/165,305, entitled METHODS FOR IDENTIFYING FACTORS FOR DIFFERENTIATING DEFINITIVE ENDODERM, filed Jun. 23, 2005; U.S. Provisional Patent Application No. 60/736,598, entitled MARKERS OF DEFINITIVE ENDODERM, filed Nov. 14, 2005 and U.S. patent application Ser. No. 11/474,211, entitled PREPRIMITIVE STREAK AND MESENDODERM CELLS, filed Jun. 23, 2006, the disclosures of which are incorporated herein by reference in their entireties.

### BRIEF DESCRIPTION OF THE DRAWINGS

FIG. 1 is a schematic of the early cell fate choices available to a pluripotent ESC. Four initial cell fates that an ESC may give rise to are shown: trophectoderm (TE), extraembryonic endoderm (ExE), mesendoderm (ME) and ectoderm (E). Also indicated on the schematic are differentiation factors (which include, but are not limited to bone morphogenic protein 4 (BMP4) SU5402 (SU), follistatin (FST), noggin (NOG) and Activin A (ActA)) that create unique signaling environments that control hESC lineage specification to the four initial fates. Exemplary markers for defining each cell fate are CDX2, SOX7, BRA, Wnt3A, PAX6 and SOX1.

FIGS. 2A-H are bar charts which demonstrate the effect of growth factor conditions on cell fate as evidenced by specific patterns of gene expression of markers for mesendoderm (Brachyury and Wnt3, panels A and B), ectoderm (PAX6 and SOX1, panels C and D), extraembryonic endoderm (SOX7, panel E), trophectoderm (CDX2, panel F) and definitive endoderm (SOX17 and GSC, panels G and H). Treatment of hES cells with either activin A (ActA), noggin (Nog)+follistatin (Fol), or BMP4 (BMP)+SU5402 (SU) results in drastically different populations. BMP+SU treatment results in significant differentiation to extra-embryonic endoderm as indicated by SOX7 expression. Noggin+follistatin treatment results in neural ectoderm differentiation as indicated by PAX6 expression. Additionally, the presence of high levels of insulin (INS) in the media, which signals through the phosphotidylinosito1-3-kinase (PI-3-kinase or PI-3-K) signal transduction pathway, results in a decrease in the efficiency with which the hESCs differentiate down each of the 4 primary lineages as indicated by the decreased expression of each marker in the presence of insulin.

FIGS. 3A-D are bar charts showing that treatment of hESCs with the fibroblast growth factor receptor (FGFR) inhibitor SU5402 (SU) provides a generalized signaling environment whereby the ESCs make all available fate choices as shown by elevated expression of brachyury (panel A), SOX1 (panel B), SOX7 (panel C) and CDX2 (panel D). This generalized differentiation is also inhibited strongly by the presence of insulin (SU+1) as shown in panels A-D.

FIGS. 4A-B are fluorescent micrographs showing immunocytochemical analysis of pluripotent cells treated with follistatin and noggin, either without (Panel A) or with (Panel B) the addition of insulin. Following treatment, cells were stained with antibodies against SOX17 and PAX6.

FIG. 5A-D are fluorescent micrographs showing immunocytochemical analysis of pluripotent cells treated with Activin A (Panels A and C) or a noggin and follistatin combination (Panels B and D), either with (Panels A and B) or without (Panels C and D) the addition of insulin. Following treatment, cells were stained with antibodies against SOX17 and OCT4.

### DETAILED DESCRIPTION

Human embryonic stem cells (hESCs) hold great promise for cell therapeutic application to a variety of degenerative disease states. Therapeutic application involves the directed differentiation of hESCs to yield a more mature cell type that has the capacity to replace the functions of the cell type(s) lost during disease. Available data suggests that ESCs are approximately equivalent to either the inner cell mass of the blastocyst or the epiblast of the preimplantation stage mammalian embryo. Studies of spontaneous (non-directed) differentiation of ESCs suggest that ESCs appear to progress through the same stages of cellular specialization that are known to occur during mammalian embryogenesis. In view of the complexity of the developmental processes involved in embryogenesis, the hESC is very far removed from the mature cell types of the body. Accordingly, directing the differentiation of ESCs to more mature cell types is a multi-step process that involves sequential stages of cellular specialization to multiple intermediate progenitor cell phenotypes prior to the acquisition of the more mature phenotypes and functions that are useful in the treatment of disease.

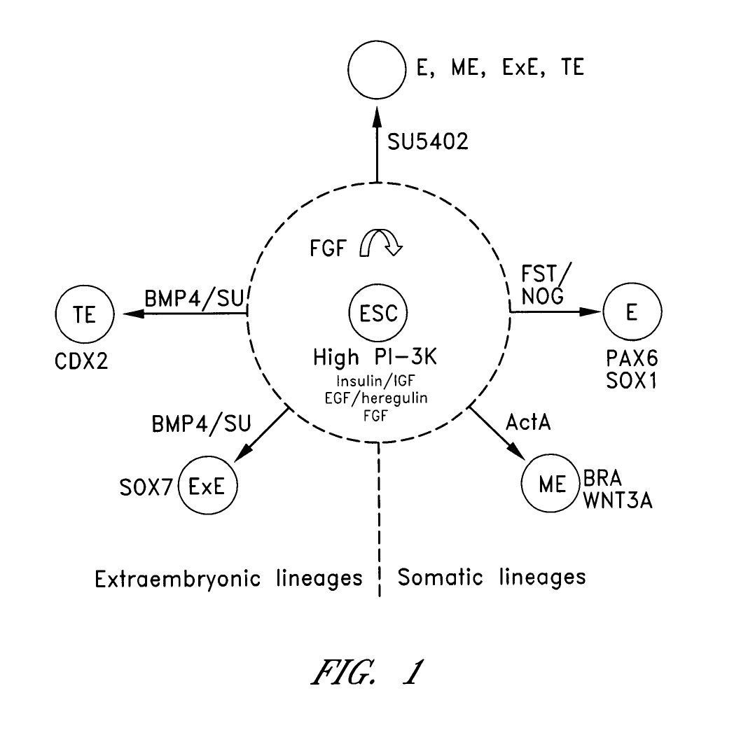

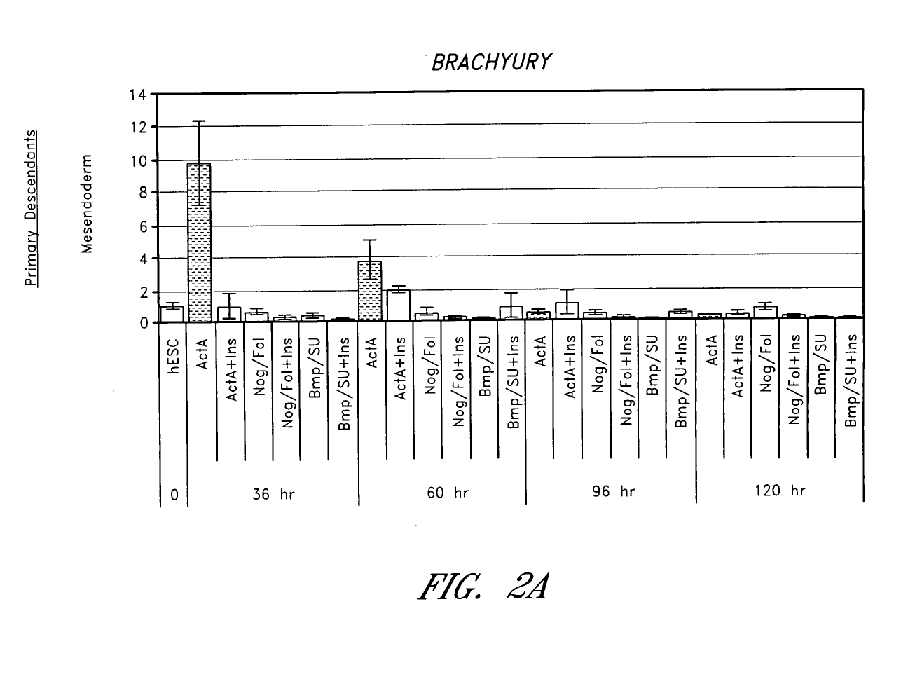

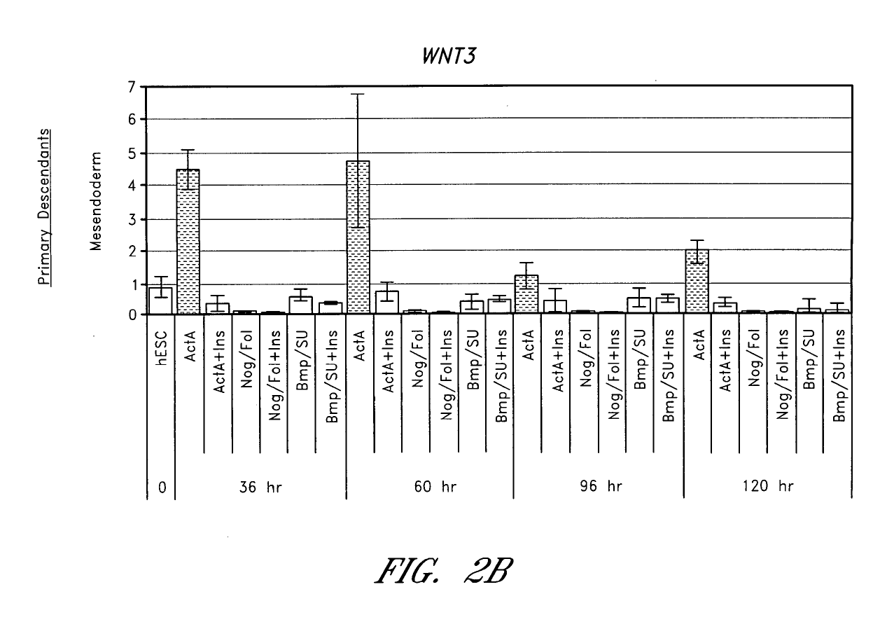

Harnessing the differentiation potential of hESCs through directed differentiation to specific mature cell type(s) has thus far been a very difficult task. In order to efficiently direct hESC differentiation to various therapeutically useful cell types it is desirable to exert exquisite control over the first fate choices available to the ESC. Since ESCs are approximately equivalent to the inner cell mass and based on the currently understood paradigms of developmental biology, it follows that the ESC has four differentiation events immediately available to it. As graphically represented in FIG. 1, the four cell fates that an ESC may give rise to are: trophectoderm, primitive endoderm, mesendoderm and ectoderm. These early cell fates choices are also represented in Table 1.

TABLE 1
List of cell fates available to differentiating ESC
and lineage relationship of more mature derivatives

| Source | Primary Descendant | Secondary Descendant |
|---|---|---|
| ESC | Trophectoderm | Mural trophoblast |
| | | Polar trophoblast |
| | Primitive endoderm | Visceral endoderm |
| | | Parietal Endoderm |
| | Ectoderm | Neural ectoderm |
| | | Non-neural ectoderm |
| | Mesendoderm | Definitive endoderm |
| | | Mesoderm |
| | | Extra-embryonic mesoderm |

Subsequent to these initial steps in lineage specification, the secondary descendants transition through multiple intermediate phenotypes before differentiating to cell types having a high therapeutic value. During embryogenesis, these transitions are orchestrated through dynamic changes in the cellular environment. Recapitulation of these dynamic environments in vitro requires that the differentiation events are reasonably rapid and synchronous, ultimately producing cell populations of similar progenitor cell phenotype that can then respond to the next set of signals applied to the culture. Therefore, it is extremely useful to develop methods for synchronous and efficient differentiation of ESCs in vitro in order to effectively facilitate multi-step directed cell differentiation.

A crucial stage in early human development termed gastrulation occurs 2-3 weeks after fertilization. Gastrulation is extremely significant because it is at this time that the three primary germ layers are first specified and organized (Lu et al., 2001; Schoenwolf and Smith, 2000). The ectoderm is responsible for the eventual formation of the outer coverings of the body and the entire nervous system whereas the heart, blood, bone, skeletal muscle and other connective tissues are derived from the mesoderm. Definitive endoderm is defined as the germ layer that is responsible for formation of the entire gut tube which includes the esophagus, stomach and small and large intestines, and the organs which derive from the gut tube such as the lungs, liver, thymus, parathyroid and thyroid glands, gall bladder and pancreas (Grapin-Botton and Melton, 2000; Kimelman and Griffin, 2000; Tremblay et al., 2000; Wells and Melton, 1999; Wells and Melton, 2000). A very important distinction should be made between the definitive endoderm and the completely separate lineage of cells termed primitive endoderm. The primitive endoderm is primarily'responsible for formation of extra-embryonic tissues, mainly the parietal and visceral endoderm portions of the placental yolk sac and the extracellular matrix material of Reichert's membrane.

In vivo analyses of the formation of definitive endoderm, such as the studies in Zebrafish and Xenopus by Conlon et al., 1994; Feldman et al., 1998; Zhou et al., 1993; Aoki et al., 2002; Dougan et al., 2003; Tremblay et al., 2000; Vincent et al., 2003; Alexander et al., 1999; Alexander and Stainier, 1999; Kikuchi et al., 2001; Hudson et al., 1997 and in mouse by Kanai-Azuma et al., 2002 lay a foundation for how one might attempt to approach the development of a specific germ layer cell type in the culture dish using human embryonic stem cells.

There are at least two aspects associated with in vitro ESC culture that pose major obstacles in the attempt to recapitulate development in the culture dish. First, organized germ layer or organ structures are not produced. Second, the timing of gene expression patterns dictates the movement down a specific developmental pathway. Regarding the first obstacles, the majority of germ layer and organ specific genetic markers are expressed in a heterogeneous fashion in the differentiating hESC culture system. As such, it is difficult to evaluate formation of a specific tissue or cell type due to this lack of organ specific boundaries. Almost all genes expressed in one cell type within a particular germ layer or tissue type are expressed in other cells of different germ layer or tissue types as well. Without specific boundaries there is considerably less means to assign gene expression specificity with a small sample of 1-3 genes. Therefore, one typically needs to examine considerably more genes, some of which should be present as well as some that should not be substantially expressed in the particular cell type of the organ or tissue of interest.

To further complicate matters, it should be noted that stem cell differentiation in vitro is rather asynchronous, likely considerably more so than in vivo. As such, one group of cells may be expressing genes associated with gastrulation, while another group may be starting final differentiation. Furthermore, manipulation of hESC monolayers or embryoid bodies (EBs) with or without exogenous factor application may result in profound differences with respect to overall gene expression pattern and state of differentiation. For these reasons, the application of exogenous factors should be timed according to gene expression patterns within a heterogeneous cell mixture in order to efficiently move the culture down a specific differentiation pathway. It is also beneficial to consider the morphological association of the cells in the culture vessel. The ability to uniformly influence hESCs when formed into so called embryoid bodies may be less optimal than hESCs grown and differentiated as monolayers and or hESC colonies in the culture vessel.

As an effective way to deal with the above-mentioned problems of heterogeneity and asynchrony, some embodiments of the present invention contemplate methods for producing enriched cell populations of primary descendants and other early stage precursor cells derived from hESCs.

### DEFINITIONS

It will be appreciated that the numerical ranges expressed herein include the endpoints set forth and describe all integers between the endpoints of the stated numerical range.

As used herein, “multipotent” or “multipotent cell” refers to a cell type that can give rise to a limited number of other particular cell types. Multipotent cells are committed to one or more embryonic cell fates, and thus, in contrast to pluripotent cells, cannot give rise to each of the three embryonic cell lineages as well as extraembryonic cells.

In some embodiments, “pluripotent cells” are used as the starting material for pancreatic islet hormone-expressing cell differentiation. By “pluripotent” is meant that the cell can give rise to each of the three embryonic cell lineages as well as extraembryonic cells. Pluripotent cells, however, may not be capable producing an entire organism.

In certain embodiments, the pluripotent cells used as starting material are stem cells, including human embryonic stem cells. As used herein, “embryonic” refers to a range of developmental stages of an organism beginning with a single zygote and ending with a multicellular structure that no longer comprises pluripotent or totipotent cells other than developed gametic cells. In addition to embryos derived by gamete fusion, the term “embryonic” refers to embryos derived by somatic cell nuclear transfer.

By “conditioned medium” is meant, a medium that is altered as compared to a base medium. For example, the conditioning of a medium may cause molecules, such as nutrients and/or growth factors, to be added to or depleted from the original levels found in the base medium. In some embodiments, a medium is conditioned by allowing cells of certain types to be grown or maintained in the medium under certain conditions for a certain period of time. For example, a medium can be conditioned by allowing hESCs to be expanded, differentiated or maintained in a medium of defined composition at a defined temperature for a defined number of hours. As will be appreciated by those of skill in the art, numerous combinations of cells, media types, durations and environmental conditions can be used to produce nearly an infinite array of conditioned media.

When used in connection with cell cultures and/or cell populations, the term “portion” means any non-zero amount of the cell culture or cell population, which ranges from a single cell to the entirety of the cell culture or cells population. In preferred embodiments, the term “portion” means at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94% or at least 95% of the cell culture or cell population.

With respect to cells in cell cultures or in cell populations, the term “substantially free of” means that the specified cell type of which the cell culture or cell population is free, is present in an amount of less than about 10%, less than about 9%, less than about 8%, less than about 7%, less than about 6%, less than about 5%, less than about 4%, less than about 3%, less than about 2% or less than about 1% of the total number of cells present in the cell culture or cell population.

As used herein, “produced from hESCs,” “derived from hESCs,” “differentiated from hESCs” and equivalent expressions refer to the production of a differentiated cell type from hESCs in vitro rather than in vivo.

As used herein, “primary descendant” refers to the immediate progeny derived after the initial differentiation event of hESCs. Primary descendants may include cells of the trophectoderm, primitive endoderm (extraembryonic endoderm), ectoderm, and mesendoderm cell fates.

As used herein “secondary descendant” refers to intermediate cell types derived from the differentiation events of primary descendants of hESCs, as defined above.

As used herein, the term “inhibitor of the PI-3-kinase pathway” or “PI-3-K pathway inhibitor” refers to any molecule or compound that limits, decreases or inhibits the activity of PI-3-kinase in a cell contacted with the inhibitor. In some embodiments, term “inhibitor of the PI-3-kinase pathway” or “PI-3-K pathway inhibitor” refers to any molecule or compound that limits, decreases or inhibits the activity of at least one molecule downstream of PI-3-kinase in a cell contacted with the inhibitor.

In some embodiments of the present invention, the pluripotent cells are contacted with an effective amount of the inhibitor of the PI-3-kinase pathway. As used in connection with PI-3-K inhibitors, the term “effective amount” refers to the concentration of inhibitor that is sufficient to decrease the activity of PI-3-kinase or at least one molecule downstream of PI-3-kinase in a pluripotent cell that has been contacted with the inhibitor and a differentiation factor so as to effect differentiation of a pluripotent cell towards a trophoblast, extraembryonic endoderm, or ectoderm cell fate.

As used herein, the term “activator of the PI-3-kinase pathway” or “PI-3-K pathway activator” refers to any molecule or compound that promotes, increases or stimulates the activity of PI-3-kinase in a cell contacted with the activator. In some embodiments, term “activator of the PI-3-kinase pathway” or “PI-3-K pathway activator” refers to any molecule or compound that promotes, increases or stimulates the activity of at least one molecule downstream of PI-3-kinase in a cell contacted with the activator.

As used in connection with PI-3-K activators, an term “effective amount” refers to the concentration of activator that is sufficient to increase the activity of PI-3-kinase or at least one molecule downstream of PI-3-kinase in a pluripotent cell that has been contacted with the activator. In some embodiments of the present invention, human pluripotent cells, such as hESCs, are maintained, grown or differentiated in a medium that limits, decreases or inhibit PI-3-K signaling. In such embodiments, the medium in which the cells are maintained, grown or differentiated, lacks an “effective amount” of a PI-3-K activator such as, serum, insulin, insulin analogs, insulin-like growth factors, insulin-like growth factor analogs and/or insulin mimetics.

As used herein, “substantial concentration,” “substantial amount,” and equivalent expressions refer to a concentration of a molecule that is sufficient to produce effective cell signaling. For example, in some embodiments, a substantial concentration with regard to PI-3-K signaling is a concentration of a molecule, such as a PI-3-K activator, that is sufficient to produce levels of PI-3-K signaling in a cell that cause a reduced level of differentiation of pluripotent cells to any particular cell fate. In some embodiments, the medium in which cells are maintained, grown or differentiated, lacks a “substantial concentration” or a “substantial amount” of an activator of the PI-3-kinase pathway such as, insulin, insulin analogs, insulin-like growth factors, insulin-like growth factor analogs and/or insulin mimetics.

As used herein, “differentiation factor,” “growth factor,” “differentiation signaling factor,” and equivalent expressions refer to any molecule that promotes growth or differentiation of a pluripotent or multipotent cell.

By “FGF family growth factor” or “member of the fibroblast growth factor family” is meant an FGF selected from the group consisting of FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22 and FGF23. In some embodiments, “FGF family growth factor” or “member of the fibroblast growth factor family” means any growth factor having homology and/or function similar to a known member of the fibroblast growth factor family.

As used herein, “expression” refers to the production of a material or substance as well as the level or amount of production of a material or substance. Thus, determining the expression of a specific marker refers to detecting either the relative or absolute amount of the marker that is expressed or simply detecting the presence or absence of the marker.

In some embodiments, hESCs can be derived from a “preimplantation embryo.” As used herein, “preimplantation embryo” refers to an embryo between the stages of fertilization and implantation. Thus, a preimplantation embryo has not progressed beyond the blastocyst stage. Implantation generally takes place 7-8 days after fertilization. However, implantation may take place about 5, about 6, about 7, about 8, about 9, about 10, about 11, about 12, about 13, about 14 or greater than about 14 days after fertilization.

As used herein, “marker” refers to any molecule that can be observed or detected. For example, a marker can include, but is not limited to, a nucleic acid, such as a transcript of a specific gene, a polypeptide product of a gene, a non-gene product polypeptide, a glycoprotein, a carbohydrate, a glycolipid, a lipid, a lipoprotein or a small molecule (for example, molecules having a molecular weight of less than 10,000 amu).

For most markers described herein, the official Human Genome Organisation (HUGO) gene symbol is provided. Such symbols, which are developed by the HUGO Gene Nomenclature Committee, provide unique abbreviations for each of the named human genes and gene products. These gene symbols are readily recognized and can easily be associated with a corresponding unique human gene and/or protein sequence by those of ordinary skill in the art.

In accordance with the HUGO designations, the following gene symbols are defined as follows: GHRL—ghrelin; IAPP—islet amyloid polypeptide; INS—insulin; GCG—glucagon; ISL1—ISL1 transcription factor; PAX6—paired box gene 6; PAX4—paired box gene 4; NEUROG3—neurogenin 3 (NGN3); NKX2-2—NKX2 transcription factor related, locus 2 (NKX2.2); NKX6-1—NKX6 transcription factor related, locus 1 (NKX6.1); IPF1—insulin promoter factor 1 (PDX1); ONECUT1—one cut domain, family member 1 (HNF6); HLXB9—homeobox B9 (HB9); TCF2—transcription factor 2, hepatic (HNF1b); FOXA1—forkhead box A1; HGF—hepatocyte growth factor; IGF1—insulin-like growth factor 1; POU5F1—POU domain, class 5, transcription factor 1 (OCT4); NANOG—Nanog homeobox; SOX2—SRY (sex determining region Y)-box 2; CDH1—cadherin 1, type 1, E-cadherin (ECAD); T—brachyury homolog (BRACH); FGF4—fibroblast growth factor 4; WNT3—wingless-type MMTV integration site family, member 3; SOX17—SRY (sex determining region Y)-box 17; GSC—goosecoid; CER)—(cerberus 1, cysteine knot superfamily, homolog (CER); CXCR4—chemokine (C-X-C motif) receptor 4; FGF17-fibroblast growth factor 17; FOXA2—forkhead box A2; SOX7—SRY (sex determining region Y)-box 7; SOX1—SRY (sex determining region Y)-box 1; AFP—alpha-fetoprotein; SPARC—secreted protein, acidic, cysteine-rich (osteonectin); and THBD thrombomodulin (TM); HAND1—heart and neural crest derivatives expressed 1; CDX2—caudal type homeobox transcription factor 2; EOMES—eomesodermin homolog; ESX1-extraembryonic, spermatogenesis, homeobox 1 homolog (ESXL1); KRT18—keratin 18; PSG3—pregnancy specific beta-1-glycoprotein 3; SFXN5—sideroflexin 5; DLX3—distal-less homeobox 3; SPARC—secreted protein, acidic, cysteine-rich (osteonectin); HOXB1—homeobox B1; LHX5—LIM homeobox 5; MEIS1—myeloid ecotropic viral integration site 1 homolog; OTX 1—orthodenticle homolog 1.

The following provides the full gene names corresponding to non-HUGO gene symbols as well as other abbreviations that may be used herein: SS—somatostatin (SOM); PP—pancreatic polypeptide; C-peptide—connecting peptide; Ex4—exendin 4; NIC—nicotinamide and DAPT—N-[N-(3,5-difluorophenacetyl)-L-alanyl]-S-phenylglycine t-butyl ester; RA—retinoic acid; RPMI—Roswell Park Memorial Institute mediumH CMRL—Connaught Medical Research Labs medium; FBS—fetal bovine serum.

### Human Embryonic Stem Cells

A preferred method for deriving trophectoderm, extraembryonic endoderm and ectoderm cells utilizes human embryonic stem cells (hESCs) as the starting material. Generally, hESCs can be derived from a human preimplantation embryo. In some processes, the hESCs can be derived from the morula, embryonic inner cell mass or the embryonic gonadal ridges. Human embryonic stem cells can be maintained in culture in a pluripotent state without substantial differentiation using methods that are known in the art. Such methods are described, for example, in U.S. Pat. Nos. 5,453,357, 5,670,372, 5,690,926 5,843,780, 6,200,806 and 6,251,671 the disclosures of which are incorporated herein by reference in their entireties.

In some processes, hESCs are maintained on a feeder layer. In such processes, any feeder layer which allows hESCs to be maintained in a pluripotent state can be used. One commonly used feeder layer for the cultivation of human embryonic stem cells is a layer of mouse fibroblasts. More recently, human fibroblast feeder layers have been developed for use in the cultivation of hESCs (see US Patent Application No. 2002/0072117, the disclosure of which is incorporated herein by reference in its entirety). Alternative processes permit the maintenance of pluripotent hESC without the use of a feeder layer. Methods of maintaining pluripotent hESCs under feeder-free conditions have been described in US Patent Application No. 2003/0175956, the disclosure of which is incorporated herein by reference in its entirety.

The human embryonic stem cells used herein can be maintained in culture either with or without serum. In some embryonic stem cell maintenance procedures, serum replacement is used. In others, serum free culture techniques, such as those described in US Patent Application No. 2003/0190748, the disclosure of which is incorporated herein by reference in its entirety, are used.

Stem cells are maintained in culture in a pluripotent state by routine passage until it is desired that they be differentiated into the desired primary descendant lineage, and then ultimately to more mature derivative cells.

Embodiments of the present invention relate to novel, defined processes for the production of trophectoderm, primitive endoderm or ectoderm in culture by differentiating pluripotent cells, such as stem cells, into multipotent, primary descendant cells such as trophectoderm, primitive endoderm or ectoderm cells. In certain preferred embodiments, the trophectoderm, primitive endoderm or ectoderm cells are derived from hESCs. Such processes can provide the basis for efficient production of human ectodermal derived tissues (such as neurons and skin cells), extraembryonic endoderm derived tissues (such as the parietal and visceral endoderm portions of the placental yolk sac and the extracellular matrix material of Reichert's membrane) and trophectoderm derived tissues (such as placenta). For example, production of ectoderm may be the first step in differentiation of a stem cell to a functional neuron. To obtain useful quantities of functional neurons, high efficiency of differentiation is desirable for each of the differentiation steps that occur prior to reaching the neural cell fate. Since differentiation of stem cells to ectoderm cells represents one of the earliest steps towards the production of functional neurons, high efficiency of differentiation at this step is particularly desirable.

In view of the desirability of efficient differentiation of pluripotent cells to the trophectoderm, primitive endoderm or ectoderm cells, some aspects of the present invention relate to in vitro methodology that results in approximately 5% to approximately 95% conversion of pluripotent cells to the trophectoderm, primitive endoderm or ectoderm cells. Typically, such methods encompass the application of culture and growth factor conditions in a defined and temporally specified fashion. Further enrichment of the cell population for the trophectoderm, primitive endoderm or ectoderm cells can be achieved by isolation and/or purification of the trophectoderm, primitive endoderm or ectoderm cells from other cells in the population by using a reagent that specifically binds to the trophectoderm, primitive endoderm or ectoderm cells. As such, aspects of the present invention relate to trophectoderm, primitive endoderm or ectoderm cells as well as methods for producing and isolating and/or purifying such cells.

In order to determine the amount of trophectoderm, primitive endoderm or ectoderm cells in a cell culture or cell population, a method of distinguishing that particular cell type from the other cells in the culture or in the population is desirable. Accordingly, certain embodiments of the present invention relate to cell markers whose presence, absence and/or relative expression levels are at least partially specific for trophectoderm, primitive endoderm or ectoderm and methods for detecting and determining the expression of such markers

In some embodiments of the present invention, the presence, absence and/or level of expression of a marker is determined by quantitative PCR (Q-PCR). For example, the amount of transcript produced by certain genetic markers, such as HAND1, Eomes, MASH2, ESXL1, HCG, KRTI8, PSG3, SFXN5, DLX3, PSX1, ETS2, ERRB, ZIC1, cytokeratin, FGF5, HOXB1, LHX5, MASH1, MEIS1, OTX1, SOX1, PAX6, SOX17, CXCR4, OCT4, SPARC, AFP, TM, SOX7 and other markers described herein is determined by quantitative Q-PCR. In other embodiments, immunohistochemistry is used to detect the proteins expressed by the above-mentioned genes. In still other embodiments, Q-PCR and immunohistochemical techniques are both used to identify and determine the amount or relative proportions of such markers.

By using methods, such as those described herein, to determine the expression of one or more appropriate markers, it is possible to identify cell cultures comprising trophectoderm, primitive endoderm and/or ectoderm. Furthermore, in some embodiments, it is possible to determine the proportion of trophectoderm, primitive endoderm or ectoderm cells in a cell culture or cell population.

Further aspects of the present invention relate to cell cultures comprising trophectoderm, primitive endoderm or ectoderm as well as cell populations enriched in trophectoderm, primitive endoderm or ectoderm cells. As such, certain embodiments relate to cell cultures which comprise trophectoderm, primitive endoderm or ectoderm cells, wherein at least about 5% to about 95% of the cells in culture are trophectoderm, primitive endoderm or ectoderm cells. A preferred embodiment relates to cell cultures comprising human cells, wherein at least about 5% to about 95% of the human cells in culture are trophectoderm, primitive endoderm or ectoderm cells. Because the efficiency of the differentiation procedure can be adjusted by modifying certain parameters, which include but are not limited to, cell growth conditions, differentiation factor concentrations and the timing of culture steps, the differentiation procedures described herein can result in at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or greater than about 95% conversion of pluripotent cells to trophectoderm, primitive endoderm or ectoderm. In other embodiments of the present invention, conversion of a pluripotent cell population, such as a stem cell population, to substantially pure trophectoderm, primitive endoderm or ectoderm cell populations is contemplated.

The compositions and methods described herein have several useful features. For example, the cell cultures and cell populations that differentiate from ESCs, such as trophectoderm, primitive endoderm or ectoderm have uses in various industrial fields including, but not limited to, drug discovery, drug development and testing, toxicology, and the production of cells for therapeutic purposes. Additionally, the methods for producing such cell cultures and cell populations are useful for modeling the early stages of human development. Since compositions comprising one or more of the primary cell fates described herein serves as the source for only a limited number of tissues, such compositions can be used in the development of pure tissue or cell types, which can then be used in cell therapy or drug screening applications. For example, the particular compositions and methods described herein can serve as the basis for the derivation of cells that are useful in therapeutic intervention in disease states, such as Parkinson's disease, amyotrophic lateral sclerosis, Alzheimer's disease, skin disorders and other disease types.

### Production of Trophectoderm From Pluripotent Cells

Human pluripotent cells are maintained in culture in a pluripotent state by routine passage until it is desired that they be differentiated into trophectoderm. In some embodiments, differentiation to trophectoderm is achieved by providing to the pluripotent cell culture a differentiation factor in an amount sufficient to promote differentiation to trophectoderm. In some embodiments, differentiation factors which are useful for the production of trophectoderm are selected from the BMP subgroup. In preferred embodiments of the differentiation methods described herein, the differentiation factor is BMP4. In certain embodiments of the present invention, a BMP differentiation factor in combination with one or more other differentiation factors can be used. In certain embodiments, the FGFR inhibitor SU5402 is provided alone or in combination with a BMP, such as BMP4, in order to further promote differentiation to the trophectoderm lineage.

With respect to some of the embodiments of differentiation methods described herein, one or more of the above-mentioned differentiation factors are provided to the cells so that the differentiation factors are present in the cultures at concentrations sufficient to promote differentiation of at least a portion of the human pluripotent cells to trophectoderm. In some embodiments of the present invention, the above-mentioned differentiation factors are present in the cell culture at a concentration of at least about 5 ng/ml, at least about 10 ng/ml, at least about 25 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, at least about 1000 ng/ml, at least about 2000 ng/ml, at least about 3000 ng/ml, at least about 4000 ng/ml, at least about 5000 ng/ml or more than about 5000 ng/ml. In certain embodiments, the FGFR inhibitor SU5402 is provided alone or in combination with a BMP differentiation factor and is present in the cell culture at a concentration of at least about 0.01 μM, at least about 0.1 μM, at least about 0.5 μM, at least about 1 μM, at least about 2 μM, at least about 5 μM, at least about 10 μM, at least about 20 μM, at least about 30 μM, at least about 40 μM, at least about 50 μM, at least about 100 μM, at least about 200 μM, at least about 500 μM or at least about 1 mM.

In certain embodiments of the present invention, the above-mentioned differentiation factors are removed from the cell culture subsequent to their addition. For example, the differentiation factors can be removed within about one day, about two days, about three days, about four days, about five days, about six days, about seven days; about eight days, about nine days or about ten days after their addition. In a preferred embodiment, the differentiation factors are removed from about three to about five days after their addition.

In some embodiments of the present invention, human pluripotent cells, such as hESCs, can be differentiated to trophectoderm cells containing reduced serum or no serum. The customary level of serum in culture medium for maintaining cell survival is 10% (v/v). Among other things, serum promotes signaling of the PI-3-K pathway. It has been surprisingly discovered that PI-3-kinase signaling can restrict the potential of hESCs to differentiate to certain primary cell lineages. As such, in certain embodiments, the level of serum in the culture medium is reduced below the customary concentration in order to reduce PI-3-K signaling and promote differentiation. In some embodiments, the culture medium comprises less than about 10% (v/v) serum and lacks serum replacement. In certain embodiments of the present invention, serum concentrations can range from about 0.01% v/v to about 10% v/v. For example, in certain embodiments, the serum concentration of the medium can be less than about 0.01% (v/v), less than about 0.05% (v/v), less than about 0.1% (v/v), less than about 0.2% (v/v), less than about 0.3% (v/v), less than about 0.4% (v/v), less than about 0.5% (v/v), less than about 0.6% (v/v), less than about 0.7% (v/v), less than about 0.8% (v/v), less than about 0.9% (v/v), less than about 1% (v/v), less than about 2% (v/v), less than about 3% (v/v), less than about 4% (v/v), less than about 5% (v/v), less than about 6% (v/v), less than about 7% (v/v), less than about 8% (v/v), less than about 9% (v/v) and less than about 10% (v/v). In some embodiments, the hESCs are differentiated to trophectoderm cells without serum and without serum replacement. In still other embodiments, the hESCs are differentiated to trophectoderm cells in the presence of about 2% serum or less.

In some embodiments, the serum concentration is increased over time to promote survival and growth of the differentiating cells in culture. Thus, even though human pluripotent cells, such as hESCs, can be contacted with a culture medium containing reduced serum or no serum initially in order to limit PI-3-kinase signaling and promote differentiation to trophectoderm lineage, in certain embodiments the serum concentration is increased over time. In such embodiments, the serum concentration is increased after about 1 day from initially contacting the cells with culture medium containing reduced serum or no serum. In other embodiments, the serum concentration can be increased after about 0.5 days, about 1 day, about 2 days, about 3 days, about 4 days, about 5 days, or after about 6 days after the initial contacting step. After about 1 day after contacting the cells with culture medium containing reduced serum or no serum, the concentration of serum present in the culture medium can be about 0.01% (v/v), about 0.05% (v/v), about 0.1% (v/v), about 0.2% (v/v), about 0.3% (v/v), about 0.4% (v/v), about 0.5% (v/v), about 0.6% (v/v), about 0.7% (v/v), about 0.8% (v/v), about 0.9% (v/v), about 1% (v/v), about 2% (v/v), about 3% (v/v), about 4% (v/v), about 5% (v/v), about 6% (v/v), about 7% (v/v), about 8% (v/v), about 9% (v/v), about 10% (v/v), about 15% (v/v) or about 20% (v/v). After about 2 days after contacting the cells with culture medium containing reduced serum or no serum, the concentration of serum present in the culture medium can be about 0.01% (v/v), about 0.05% (v/v), about 0.1% (v/v), about 0.2% (v/v), about 0.3% (v/v), about 0.4% (v/v), about 0.5% (v/v), about 0.6% (v/v), about 0.7% (v/v), about 0.8% (v/v), about 0.9% (v/v), about 1% (v/v), about 2% (v/v), about 3% (v/v), about 4% (v/v), about 5% (v/v), about 6% (v/v), about 7% (v/v), about 8% (v/v), about 9% (v/v), about 10% (v/v), about 15% (v/v) or about 20% (v/v).

In some embodiments of the present invention, cultures of human pluripotent cells, such as hESCs, can be differentiated to trophectoderm cells in medium that lacks a substantial concentration of a molecule that promotes PI-3-kinase signaling activity. Molecules that activate PI-3-kinase signaling activity are known in the art, and include, for example, insulin, insulin analogs, insulin-like growth factors, insulin-like growth factor analogs, insulin-mimetic compounds and combinations thereof. In certain embodiments, the culture medium comprises less than about 2 μg/ml insulin. In other embodiments, the culture medium comprises less than about 10 ng/ml, less than about 50 ng/ml, less than about 100 ng/ml, less than about 200 ng/ml, less than about 500 ng/ml, less than about 1 μg/ml, less than about 2 μg/ml, less than about 3 μg/ml, less than about 4 μg/ml, less than about 5 μg/ml, less than about 10 ng/ml, less than about 20 μg/ml, less than about 50 μg/ml, less than about 100 μg/ml or less than about 200 μg/ml insulin. In certain embodiments, the culture medium comprises less than about 2 μg/ml of an insulin analog. In other embodiments, the culture medium comprises less than about 10 ng/ml, less than about 50 ng/ml, less than about 100 ng/ml, less than about 200 ng/ml, less than about 500 ng/ml, less than about 1 μg/ml, less than about 2 μg/ml, less than about 3 μg/ml, less than about 4 μg/ml, less than about 5 μg/ml, less than about 10 μg/ml, less than about 20 μg/ml, less than about 50 μg/ml, less than about 100 μg/ml or less than about 200 μg/ml of an insulin analog.

In certain embodiments of the present invention, the culture medium lacks a substantial concentration of an insulin-like growth factor or insulin-like growth factor analogs. The insulin-like growth factor can be, for example, insulin-like growth factor-1 (IGF-1), insulin-like growth factor-2 (IGF-2) or any other insulin-like growth factor analogs. In certain embodiments, the culture medium comprises less than about 10 ng/ml of insulin-like growth factor-1 or insulin-like growth factor analogs. In other embodiments, the culture medium comprises less than about 0.1 ng/ml, less than about 1 ng/ml, less than about 2 ng/ml, less than about 3 ng/ml, less than about 4 ng/ml, less than about 5 ng/ml, less than about 6 ng/ml, less than about 7 ng/ml, less than about 8 ng/ml, less than about 9 ng/ml, less than about 10 ng/ml, less than about 20 ng/ml, less than about 50 ng/ml, less than about 100 ng/ml or less than about 200 ng/ml of insulin-like growth factor or insulin-like growth factor analogs.

In certain embodiments, the culture medium lacks a substantial concentration of an insulin mimetic compound. The insulin mimetic compound can be, for example vanadium(IV) oxo-bis(maltolato) (BMOV), ZnCl₂, bis(maltolato)zinc(II), zinc(II) complexes, vanadyl(IV) complexes, and the like. In certain embodiments, the culture medium comprises less than about 2 μg/ml of an insulin mimetic compound. In other embodiments, the culture medium comprises less than about 10 ng/ml, less than about 50 ng/ml, less than about 100 ng/ml, less than about 200 ng/ml, less than about 500 ng/ml, less than about 1 μg/ml, less than about 2 μg/ml, less than about 3 μg/ml, less than about 4 μg/ml, less than about 5 μg/ml, less than about 10 μg/ml, less than about 20 μg/ml, less than about 50 μg/ml, less than about 100 μg/ml or less than about 200 μg/ml of an insulin mimetic compound. Insulin mimetic compounds are known in the art and their synthesis, pharmacology, and activity have been described (Cocco et al., 2006; Sakurai and Adachi, 2005; Mehdi et al., 2006, each of which is hereby incorporated by reference in its entirety).

In a preferred embodiment of the present invention, hESCs are differentiated to trophectoderm cells in a medium comprising less than about 2% serum, less than about 2 μg/ml insulin, less than about 2 μg/ml of an insulin analog, less than less than about 10 ng/ml of an insulin-like growth factor, less than 10 ng/ml of an insulin-like growth factor analog and/or less than 2 μg/ml of an insulin mimetic.

In some embodiments, the pluripotent cells are treated with an effective amount of an inhibitor of the PI-3-kinase pathway. Examples of PI-3-kinase pathway inhibitors include PI-3-kinase antagonists, antagonists of the PI-3-kinase signal transduction cascade, compounds that decrease the synthesis or expression of endogenous PI-3-kinase, compounds that decrease release of endogenous PI-3-kinase, and compounds that inhibit activators of PI-3-kinase activity. In certain embodiments of the foregoing, the inhibitor is selected from the group consisting of Rapamycin, LY 294002, wortmannin, lithium chloride, Akt inhibitor I, Akt inhibitor II (SH-5), Akt inhibitor III (SH-6), NL-71-101, and mixtures of the foregoing. Akt inhibitor I, II, Akt III, and NL-71-101 are commercially available from Calbiochem. In other embodiments, the inhibitor is selected from the group consisting of Rapamycin and LY 294002. In a further embodiment, the inhibitor comprises LY 294002. In another embodiment, the inhibitor comprises Akt1-II. In other embodiments, the inhibitor is a molecule that inhibits an upstream component of the PI-3-kinase signaling pathway. In particular embodiments of the foregoing, the inhibitor is an inhibitor of an IGF or FGF receptor. It will also be understood that combinations of inhibitors may be used to elicit the desired effect.

In one embodiment, the inhibitor is Rapamycin. In certain embodiments, Rapamycin is initially present at a concentration of approximately 0.1 nM to approximately 500 nM, approximately 0.5 nM to approximately 250 nM, approximately 1.0 nM to approximately 150 nM, or approximately 1.5 nM to approximately 30 nM. In another embodiment, the inhibitor is LY 294002. In certain embodiments, LY 294002 is initially present at a concentration of approximately 1 μM to approximately 500 μM, approximately 2.5 μM to approximately 400 μM, approximately 5 μM to approximately 250 μM, approximately 10 μM to approximately 200 μM or approximately 20 μM to approximately 163 μM. In another embodiment, the inhibitor is Akt1-II. In certain embodiments, Akt1-II is initially present at a concentration of approximately 0.1 μM to approximately 500 μM, approximately 1 μM to approximately 250 approximately 5 μM to approximately 20 μM, approximately 10 μM to approximately 100 μM or approximately 40 μM.

It will be appreciated that the aforementioned inhibitors of PI-3-kinase can be added to the cells under conditions where levels of serum, insulin, insulin analogs, insulin-like growth factors, insulin-like growth factor analogs or insulin-mimetic compounds are reduced or eliminated. In other words, in certain embodiments, inhibitors of PI-3-kinase can be added to a medium that lacks a substantial concentration or effective amount of one or more PI-3-kinase activators such as, serum, insulin, insulin analogs, insulin-like growth factors, insulin-like growth factor analogs or insulin-mimetic compounds. Alternatively, inhibitors of PI-3-kinase can be added to the cells under conditions where levels of serum, insulin, insulin analogs, insulin-like growth factors, insulin-like growth factor analogs, insulin-mimetic compounds have not been reduced or eliminated.

In some embodiments, a cell differentiating medium or environment may be utilized to partially, terminally, or reversibly differentiate the pluripotent cells of the present invention, either prior to, during, or after contacting the pluripotent cells with at least one differentiation factor and with a culture medium that limits PI-3-kinase signaling. In accordance with one embodiment of the present invention, the medium of the cell differentiation environment may contain a variety of components including, for example, KODMEM medium (Knockout Dulbecco's Modified Eagle's Medium), DMEM, Ham's F12 medium, FBS (fetal bovine serum), FGF2 (fibroblast growth factor 2), KSR or bLIF (human leukemia inhibitory factor). The cell differentiation environment can also contain supplements such as L-Glutamine, NEAA (non-essential amino acids), P/S (penicillin/streptomycin), N2 and β-mercaptoethanol (β-ME). It is contemplated that additional factors may be added to the cell differentiation environment including, but not limited to, fibronectin, laminin, heparin, heparin sulfate, retinoic acid, members of the epidermal growth factor family (EGFs), members of the fibroblast growth factor family (FGFs) including FGF2 and/or FGF8, members of the platelet derived growth factor family (PDGFs), transforming growth factor (TGF)/bone morphogenetic protein (BMP)/growth and differentiation factor (GDF) factor family antagonists including, but not limited to, noggin, follistatin, chordin, gremlin, cerberus/DAN family proteins, ventropin, high dose activin, and amnionless. TGF/BMP/GDF antagonists could also be added in the form of TGF/BMP/GDF receptor-Fc chimeras. Other factors that may be added include molecules that can activate or inactivate signaling through Notch receptor family, including but not limited to proteins of the Delta-like and Jagged families as well as inhibitors of Notch processing or cleavage. Other growth factors may include members of the insulin like growth factor family (IGF), insulin, the wingless related (WNT) factor family, and the hedgehog factor family. Additional factors may be added to promote trophectoderm stem/progenitor proliferation and survival as well as survival and differentiation of derivatives of these progenitors.

In other embodiments, the cell differentiation environment comprises plating the cells in an adherent culture. As used herein, the terms “plated” and “plating” refer to any process that allows a cell to be grown in adherent culture. As used herein, the term “adherent culture” refers to a cell culture system whereby cells are cultured on a solid surface, which may in turn be coated with a solid substrate that may in turn be coated with another surface coat of a substrate, such as those listed below, or any other chemical or biological material that allows the cells to proliferate or be stabilized in culture. The cells may or may not tightly adhere to the solid surface or to the substrate. In one embodiment, the cells are plated on matrigel coated plates. The substrate for the adherent culture may comprise anyone or combination of polyornithine, laminin, poly-lysine, purified collagen, gelatin, extracellular matrix, fibronectin, tenascin, vitronectin, entactin, heparin sulfate proteoglycans, poly glycolytic acid (PGA), poly lactic acid (PLA), poly lactic-glycolic acid (PLGA) and feeder layers such as, but not limited to, primary fibroblasts or fibroblast cells lines. Furthermore, the substrate for the adherent culture may comprise the extracellular matrix laid down by a feeder layer, or laid down by the pluripotent human cell or cell culture.

### Monitoring the Production of Trophectoderm From Pluripotent Cells

The progression of a pluripotent human cell culture, such as an hESC culture, to trophectoderm can be monitored by determining the expression of markers characteristic of trophectoderm. In some embodiments, the expression of certain markers is determined by detecting the presence or absence of the marker. Alternatively, the expression of certain markers can determined by measuring the level at which the marker is present in the cells of the cell culture or cell population. In such embodiments, the measurement of marker expression can be qualitative or quantitative. One method of quantitating the expression markers that are produced by marker genes is through the use of quantitative PCR (Q-PCR). Methods of performing Q-PCR are well known in the art. Other methods which are known in the art can also be used to quantitate marker gene expression. For example, the expression of a marker gene product can be detected by using antibodies specific for the marker gene product of interest. In some embodiments of the present invention, the expression of marker genes characteristic of trophectoderm as well as the lack of significant expression of marker genes characteristic of hESCs and other cell types is determined.

As described further in the Examples below, a reliable marker of trophectoderm is the CDX2 gene. As such, the trophectoderm cells produced by the methods described herein express the CDX2 marker, thereby producing the CDX2 gene product. Other markers of trophectoderm are HAND1, Eomes, MASH2, ESXL1, HCG, KRT18, PSG3, SFXN5, DLX3, PSX1, ETS2, and ERRB. In some embodiments of the present invention, trophectoderm cells express the CDX2 marker at a level higher than that of the SOX17 marker, which is characteristic of definitive endoderm (see Table 2) and expressed in extraembryonic cell types. Additionally, in some embodiments, expression of the CDX2 marker is higher than the expression of the OCT4 marker, which is characteristic of hESCs. In other embodiments of the present invention, trophectoderm cells express the CDX2 marker at a level higher than that of the AFP, SPARC or Thrombomodulin (TM) markers.

It will be appreciated that CDX2 marker expression is induced over a range of different levels in trophectoderm cells depending on the differentiation conditions. As such, in some embodiments of the present invention, the expression of the CDX2 marker in trophectoderm cells or cell populations is at least about 2-fold higher to at least about 10,000-fold higher than the expression of the CDX2 marker in non-trophectoderm cells or cell populations, for example pluripotent stem cells. In other embodiments of the present invention, the expression of the CDX2 marker in trophectoderm cells or cell populations is at least about 4-fold higher, at least about 6-fold higher, at least about 8-fold higher, at least about 10-fold higher, at least about 15-fold higher, at least about 20-fold higher, at least about 40-fold higher, at least about 80-fold higher, at least about 100-fold higher, at least about 150-fold higher, at least about 200-fold higher, at least about 500-fold higher, at least about 750-fold higher, at least about 1000-fold higher, at least about 2500-fold higher, at least about 5000-fold higher, at least about 7500-fold higher or at least about 10,000-fold higher than the expression of the CDX2 marker in non-trophectoderm cells or cell populations, for example pluripotent stem cells. In some embodiments, the expression of the CDX2 marker in trophectoderm cells or cell populations is infinitely higher than the expression of the CDX2 marker in non-trophectoderm cells or cell populations, for example pluripotent stem cells.

It will be appreciated that in some embodiments of the present invention, the expression of markers selected from the group consisting of HANDL Eomes, MASH2, ESXL1, HCG, KRT18, PSG3, SFXN5, DLX3, PSX1, ETS2, and ERRB in trophectoderm cells or cell populations is increased as compared to the expression of HAND1, Eomes, MASH2, ESXL1, HCG, KRT18, PSG3, SFXN5, DLX3, PSX1, ETS2, and ERRB in non-trophectoderm cells or cell populations.

It will also be appreciated that there is a range of differences between the expression level of the CDX2 marker and the expression levels of the OCT4, SPARC, AFP, TM and/or SOX7 markers in trophectoderm cells. As such, in some embodiments of the present invention, the expression of the CDX2 marker is at least about 2-fold higher to at least about 10,000-fold higher than the expression of OCT4, SPARC, AFP, TM and/or SOX7 markers. In other embodiments of the present invention, the expression of the CDX2 marker is at least about 4-fold higher, at least about 6-fold higher, at least about 8-fold higher, at least about 10-fold higher, at least about 15-fold higher, at least about 20-fold higher, at least about 40-fold higher, at least about 80-fold higher, at least about 100-fold higher, at least about 150-fold higher, at least about 200-fold higher, at least about 500-fold higher, at least about 750-fold higher, at least about 1000-fold higher, at least about 2500-fold higher, at least about 5000-fold higher, at least about 7500-fold higher or at least about 10,000-fold higher than the expression of OCT4, SPARC, AFP, TM and/or SOX7 markers. In some embodiments, OCT4, SPARC, AFP, TM and/or SOX7 markers are not significantly (substantially) expressed in trophectoderm cells.

### Compositions Comprising Trophectoderm

Some aspects of the present invention relate to compositions, such as cell populations and cell cultures, that comprise both pluripotent cells, such as stem cells, and multipotent trophectoderm cells that can differentiate into cells of the mural or polar trophoblast. For example, using the methods described herein, compositions comprising mixtures of hESCs and trophectoderm cells can be produced. In some embodiments, compositions comprising at least about 5 trophectoderm cells for about every 95 pluripotent cells are produced. In other embodiments, compositions comprising at least about 95 trophectoderm cells for about every 5 pluripotent cells are produced. Additionally, compositions comprising other ratios of trophectoderm cells to pluripotent cells are contemplated. For example, compositions comprising at least about 1 trophectoderm cell for about every 1,000,000 pluripotent cells, at least about 1 trophectoderm cell for about every 100,000 pluripotent cells, at least about 1 trophectoderm cell for about every 10,000 pluripotent cells, at least about 1 trophectoderm cell for about every 1000 pluripotent cells, at least about 1 trophectoderm cell for about every 500 pluripotent cells, at least about 1 trophectoderm cell for about every 100 pluripotent cells, at least about 1 trophectoderm cell for about every 10 pluripotent cells, at least about 1 trophectoderm cell for about every 5 pluripotent cells, at least about 1 trophectoderm cell for about every 2 pluripotent cells, at least about 2 trophectoderm cells for about every 1 pluripotent cell, at least about 5 trophectoderm cells for about every 1 pluripotent cell, at least about 10 trophectoderm cells for about every 1 pluripotent cell, at least about 20 trophectoderm cells for about every 1 pluripotent cell, at least about 50 trophectoderm cells for about every 1 pluripotent cell, at least about 100 trophectoderm cells for about every 1 pluripotent cell, at least about 1000 trophectoderm cells for about every 1 pluripotent cell, at least about 10,000 trophectoderm cells for about every 1 pluripotent cell, at least about 100,000 trophectoderm cells for about every 1 pluripotent cell and at least about 1,000,000 trophectoderm cells for about every 1 pluripotent cell are contemplated. In some embodiments of the present invention, the pluripotent cells are human embryonic stem cells. In certain embodiments the stem cells are derived from a morula, the inner cell mass of an embryo or the gonadal ridges of an embryo. In certain other embodiments, the pluripotent cells are derived from the gonadal or germ tissues of a multicellular structure that has developed past the embryonic stage. In other embodiments, the stem cells are derived from a preimplantation embryo.

Some aspects of the present invention relate to cell cultures or cell populations comprising from at least about 5% trophectoderm cells to at least about 95% trophectoderm cells. In some embodiments the cell cultures or cell populations comprise mammalian cells. In preferred embodiments, the cell cultures or cell populations comprise human cells. For example, certain specific embodiments relate to cell cultures comprising human cells, wherein from at least about 5% to at least about 95% of the human cells are trophectoderm cells. Other embodiments of the present invention relate to cell cultures comprising human cells, wherein at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, about 95%, or greater than 95% of the human cells are trophectoderm cells. In embodiments where the cell cultures or cell populations comprise human feeder cells, the above percentages are calculated without respect to the human feeder cells in the cell cultures or cell populations.

Further embodiments of the present invention relate to compositions, such as cell cultures or cell populations, comprising human cells, such as human trophectoderm cells, wherein the expression of the CDX2 marker is greater than the expression of the OCT 4, SPARC, alpha-fetoprotein (AFP), Thrombomodulin (TM) and/or SOX7 marker in at least about 5% of the human cells. In other embodiments, the expression of the CDX2 marker is greater than the expression of the OCT4, SPARC, AFP, TM and/or SOX7 marker in at least about 10% of the human cells, in at least about 15% of the human cells, in at least about 20% of the human cells, in at least about 25% of the human cells, in at least about 30% of the human cells, in at least about 35% of the human cells, in at least about 40% of the human cells, in at least about 45% of the human cells, in at least about 50% of the human cells, in at least about 55% of the human cells, in at least about 60% of the human cells, in at least about 65% of the human cells, in at least about 70% of the human cells, in at least about 75% of the human cells, in at least about 80% of the human cells, in at least about 85% of the human cells, in at least about 90% of the human cells, in at least about 95% of the human cells or in greater than 95% of the human cells. In embodiments where the cell cultures or cell populations comprise human feeder cells, the above percentages are calculated without respect to the human feeder cells in the cell cultures or cell populations.

It will be appreciated that some embodiments of the present invention relate to compositions, such as cell cultures or cell populations, comprising human cells, such as human trophectoderm cells, wherein the expression of one or more markers selected from the group consisting of HAND1, Eomes, MASH2, ESXL1, HCG, KRT18, PSG3, SFXN5, DLX3, PSX1, ETS2, and ERRB is greater than the expression of the OCT4, SPARC, AFP, TM and/or SOX7 markers in from at least about 5% to greater than at least about 95% of the human cells. In embodiments where the cell cultures or cell populations comprise human feeder cells, the above percentages are calculated without respect to the human feeder cells in the cell cultures or cell populations.

It will be appreciated that some embodiments of the present invention relate to compositions, such as cell cultures or cell populations, comprising human cells, such as human trophectoderm cells, wherein the expression of the HAND1, Eomes, MASH2, ESXL1, HCG, KRT18, PSG3, SFXN5, DLX3, PSX1, ETS2, and ERRB markers is greater than the expression of the SOX17, CXCR4, OCT4, SPARC, AFP, TM and/or SOX7 markers in from at least about 5% to greater than at least about 95% of the human cells. In embodiments where the cell cultures or cell populations comprise human feeder cells, the above percentages are calculated without respect to the human feeder cells in the cell cultures or cell populations.

It will be appreciated that some embodiments of the present invention relate to compositions, such as cell cultures or cell populations, comprising human cells, such as human trophectoderm cells, wherein the trophectoderm cells do not substantially express SOX17 or CXCR4.

Using the methods described herein, compositions comprising trophectoderm cells substantially free of other cell types can be produced. In some embodiments of the present invention, the trophectoderm cell populations or cell cultures produced by the methods described herein are substantially free of cells that significantly express the SOX17, CXCR4, OCT4, SPARC, AFP, TM and/or SOX7 markers.

Further embodiments of the present invention relate to compositions, such as cell cultures or cell populations, comprising human cells, such as human trophectoderm cells, further comprising a culture medium which comprises less than about 10% serum and lacks serum replacement. In certain embodiments of the present invention, serum concentrations can range from about 0.01% v/v to about 10% v/v. For example, in certain embodiments, the serum concentration of the medium can be less than about 0.01% (v/v), less than about 0.05% (v/v), less than about 0.1% (v/v), less than about 0.2% (v/v), less than about 0.3% (v/v), less than about 0.4% (v/v), less than about 0.5% (v/v), less than about 0.6% (v/v), less than about 0.7% (v/v), less than about 0.8% (v/v), less than about 0.9% (v/v), less than about 1% (v/v), less than about 2% (v/v), less than about 3% (v/v), less than about 4% (v/v), less than about 5% (v/v), less than about 6% (v/v), less than about 7% (v/v), less than about 8% (v/v), less than about 9% (v/v) or less than about 10% (v/v). In certain embodiments, the culture medium lacks serum and lacks serum replacement.

Further embodiments of the present invention relate to compositions, such as cell cultures or cell populations comprising human trophectoderm cells, further comprising a culture medium which comprises less than about 2 μg/ml insulin. In other embodiments, the culture medium comprises less than about 10 ng/ml, less than about 50 ng/ml, less than about 100 ng/ml, less than about 200 ng/ml, less than about 500 ng/ml, less than about 1 μg/ml, less than about 2 μg/ml, less than about 3 μg/ml, less than about 4 μg/ml, less than about 5 μg/ml, less than about 10 μg/ml, less than about 20 μg/ml, less than about 50 μg/ml, less than about 100 μg/ml or less than about 200 μg/ml insulin. In certain embodiments, the culture medium comprises less than about 2 μg/ml of an insulin analog. In other embodiments, the culture medium comprises less than about 10 ng/ml, less than about 50 ng/ml, less than about 100 ng/ml, less than about 200 ng/ml, less than about 500 ng/ml, less than about 1 μg/ml, less than about 2 μg/ml, less than about 3 μg/ml, less than about 4 μg/ml, less than about 5 μg/ml, less than about 10 μg/ml, less than about 20 μg/ml, less than about 50 μg/ml, less than about 100 μg/ml or less than about 200 μg/ml of an insulin analog.

In certain embodiments, cell cultures or cell populations comprising human trophectoderm cells comprise a culture medium that lacks a substantial concentration of an insulin-like growth factor or insulin-like growth factor analogs. The insulin-like growth factor can be, for example, insulin-like growth factor-1 (IGF-1), insulin-like growth factor-2 (IGF-2) or insulin-like growth factor analogs. In certain embodiments, the culture medium comprises less than about 10 ng/ml of insulin-like growth factor-1 or insulin-like growth factor analogs. In other embodiments, the culture medium comprises less than about 0.1 ng/ml, less than about 1 ng/ml, less than about 2 ng/ml, less than about 3 ng/ml, less than about 4 ng/ml, less than about 5 ng/ml, less than about 6 ng/ml, less than about 7 ng/ml, less than about 8 ng/ml, less than about 9 ng/ml, less than about 10 ng/ml, less than about 20 ng/ml, less than about 50 ng/ml, less than about 100 ng/ml or less than about 200 ng/ml of insulin-like growth factor or insulin-like growth factor analogs.

In other embodiments, cell cultures or cell populations comprising human trophectoderm cells comprise a culture medium that lacks a substantial concentration of an insulin mimetic compound. The insulin mimetic compound can be, for example vanadium(IV) oxo-bis(maltolato) (BMOV), ZnCl₂, bis(maltolato)zinc(II), zinc(II) complexes, vanadyl(IV) complexes, and the like. In certain embodiments, the culture medium comprises less than about 2 μg/ml of an insulin mimetic compound. In other embodiments, the culture medium comprises less than about 10 ng/ml, less than about 50 ng/ml, less than about 100 ng/ml, less than about 200 ng/ml, less than about 500 ng/ml, less than about 1 μg/ml, less than about 2 μg/ml, less than about 3 μg/ml, less than about 4 μg/ml, less than about 5 μg/ml, less than about 10 μg/ml, less than about 20 μg/ml, less than about 50 μg/ml, less than about 100 μg/ml or less than about 200 μg/ml of an insulin mimetic compound.

### Production of Ectoderm From Pluripotent Cells

Human pluripotent cells are maintained in culture in a pluripotent state by routine passage until it is desired that they be differentiated into ectoderm. In some embodiments, differentiation to ectoderm is achieved by providing to the pluripotent cell culture a differentiation factor in an amount sufficient to promote differentiation to ectoderm. Differentiation factors which are useful for the production of ectoderm are selected from the group consisting of noggin and follistatin. In some embodiments of the differentiation methods described herein, the differentiation factor is noggin. In other embodiments of the differentiation methods described herein, the differentiation factor is follistatin. In certain embodiments of the present invention, combinations of follistatin and noggin with or without other differentiation factors may be used. In certain embodiments, the FGFR inhibitor SU5402 is provided alone or in combination with follistatin and/or noggin, in order to further promote differentiation to the ectoderm lineage.

With respect to some of the embodiments of differentiation methods described herein, one or more of the above-mentioned differentiation factors are provided to the cells so that the differentiation factors are present in the cultures at concentrations sufficient to promote differentiation of at least a portion of the human pluripotent cells to ectoderm. In some embodiments of the present invention, the above-mentioned differentiation factors are present in the cell culture at a concentration of at least about 5 ng/ml, at least about 10 ng/ml, at least about 25 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, at least about 1000 ng/ml, at least about 2000 ng/ml, at least about 3000 ng/ml, at least about 4000 ng/ml, at least about 5000 ng/ml or more than about 5000 ng/ml. In certain embodiments, the FGFR inhibitor SU5402 is provided alone or in combination with follistatin and/or noggin and is present in the cell culture at a concentration of at least about 0.01 μM, at least about 0.1 μM, at least about 0.5 μM, at least about 1 μM, at least about 2 μM, at least about 5 μM, at least about 10 μM, at least about 20 μM, at least about 30 μM, at least about 40 μM, at least about 50 μM, at least about 100 μM, at least about 200 μM, at least about 500 μM or at least about 1 mM.

In certain embodiments of the present invention, the above-mentioned growth factors are removed from the cell culture subsequent to their addition. For example, the growth factors can be removed within about one day, about two days, about three days, about four days, about five days, about six days, about seven days, about eight days, about nine days or about ten days after their addition. In a preferred embodiment, the differentiation factors are removed about four days after their addition.

In some embodiments of the present invention, human pluripotent cells, such as hESCs, can be differentiated to ectoderm cells containing reduced serum or no serum. The customary level of serum in culture medium for maintaining cell survival is 10% (v/v). Among other things, serum promotes signaling of the PI-3-K pathway. It has been surprisingly discovered that PI-3-kinase signaling can restrict the potential of hESCs to differentiate to certain primary cell lineages. As such, in certain embodiments, the level of serum in the culture medium is reduced below the customary concentration in order to reduce PI-3-K signaling and promote differentiation. In some embodiments, the culture medium comprises less than about 10% (v/v) serum and lacks serum replacement. In certain embodiments of the present invention, serum concentrations can range from about 0.01% v/v to about 10% v/v. For example, in certain embodiments, the serum concentration of the medium can be less than about 0.01% (v/v), less than about 0.05% (v/v), less than about 0.1% (v/v), less than about 0.2% (v/v), less than about 0.3% (v/v), less than about 0.4% (v/v), less than about 0.5% (v/v), less than about 0.6% (v/v), less than about 0.7% (v/v), less than about 0.8% (v/v), less than about 0.9% (v/v), less than about 1% (v/v), less than about 2% (v/v), less than about 3% (v/v), less than about 4% (v/v), less than about 5% (v/v), less than about 6% (v/v), less than about 7% (v/v), less than about 8% (v/v), less than about 9% (v/v) and less than about 10% (v/v). In some embodiments, the hESCs are differentiated to ectoderm cells without serum and without serum replacement. In still other embodiments, the hESCs are differentiated to ectoderm cells in the presence of about 2% serum or less.

In some embodiments, the serum concentration is increased over time to promote survival and growth of the differentiating cells in culture. Thus, even though human pluripotent cells, such as hESCs, can be contacted with a culture medium containing reduced serum or no serum initially in order to limit PI-3-kinase signaling and promote differentiation to ectoderm lineage, in certain embodiments the serum concentration is increased over time. In such embodiments, the serum concentration is increased after about 1 day from initially contacting the cells with culture medium containing reduced serum or no serum. In other embodiments, the serum concentration can be increased after about 0.5 days, about 1 day, about 2 days, about 3 days, about 4 days, about 5 days, or after about 6 days after the initial contacting step. After about 1 day after contacting the cells with culture medium containing reduced serum or no serum, the concentration of serum present in the culture medium can be about 0.01% (v/v), about 0.05% (v/v), about 0.1% (v/v), about 0.2% (v/v), about 0.3% (v/v), about 0.4% (v/v), about 0.5% (v/v), about 0.6% (v/v), about 0.7% (v/v), about 0.8% (v/v), about 0.9% (v/v), about 1% (v/v), about 2% (v/v), about 3% (v/v), about 4% (v/v), about 5% (v/v), about 6% (v/v), about 7% (v/v), about 8% (v/v), about 9% (v/v), about 10% (v/v), about 15% (v/v) or about 20% (v/v). After about 2 days after contacting the cells with culture medium containing reduced serum or no serum, the concentration of serum present in the culture medium can be about 0.01% (v/v), about 0.05% (v/v), about 0.1% (v/v), about 0.2% (v/v), about 0.3% (v/v), about 0.4% (v/v), about 0.5% (v/v), about 0.6% (v/v), about 0.7% (v/v), about 0.8% (v/v), about 0.9% (v/v), about 1% (v/v), about 2% (v/v), about 3% (v/v), about 4% (v/v), about 5% (v/v), about 6% (v/v), about 7% (v/v), about 8% (v/v), about 9% (v/v), about 10% (v/v), about 15% (v/v) or about 20% (v/v).

In some embodiments of the present invention, cultures of human pluripotent cells, such as hESCs, can be differentiated to ectoderm cells in medium that lacks a substantial concentration of a molecule that promotes PI-3-kinase signaling activity. Molecules that activate PI-3-kinase signaling activity are known in the art, and include, for example, insulin, insulin analogs, insulin-like growth factors, insulin-like growth factor analogs, insulin-mimetic compounds and combinations thereof. In certain embodiments, the culture medium comprises less than about 2 μg/ml insulin. In other embodiments, the culture medium comprises less than about 10 ng/ml, less than about 50 ng/ml, less than about 100 ng/ml, less than about 200 ng/ml, less than about 500 ng/ml, less than about 1 μg/ml, less than about 2 μg/ml, less than about 3 μg/ml, less than about 4 μg/ml, less than about 5 μg/ml, less than about 10 μg/ml, less than about 20 μg/ml, less than about 50 μg/ml, less than about 100 μg/ml or less than about 200 μg/ml insulin. In certain embodiments, the culture medium comprises less than about 2 μg/ml of an insulin analog. In other embodiments, the culture medium comprises less than about 10 ng/ml, less than about 50 ng/ml, less than about 100 ng/ml, less than about 200 ng/ml, less than about 500 ng/ml, less than about 1 μg/ml, less than about 2 μg/ml, less than about 3 μg/ml, less than about 4 μg/ml, less than about 5 μg/ml, less than about 10 μg/ml, less than about 20 μg/ml, less than about 50 μg/ml, less than about 100 μg/ml or less than about 200 μg/ml of an insulin analog.

In certain embodiments of the present invention, the culture medium lacks a substantial concentration of an insulin-like growth factor or insulin-like growth factor analogs. The insulin-like growth factor can be, for example, insulin-like growth factor-1 (IGF-1), insulin-like growth factor-2 (IGF-2) or any other insulin-like growth factor analogs. In certain embodiments, the culture medium comprises less than about 10 ng/ml of insulin-like growth factor-1 or insulin-like growth factor analogs. In other embodiments, the culture medium comprises less than about 0.1 ng/ml, less than about 1 ng/ml, less than about 2 ng/ml, less than about 3 ng/ml, less than about 4 ng/ml, less than about 5 ng/ml, less than about 6 ng/ml, less than about 7 ng/ml, less than about 8 ng/ml, less than about 9 ng/ml, less than about 10 ng/ml, less than about 20 ng/ml, less than about 50 ng/ml, less than about 100 ng/ml or less than about 200 ng/ml of insulin-like growth factor or insulin-like growth factor analogs.

In certain embodiments, the culture medium lacks a substantial concentration of an insulin mimetic compound. The insulin mimetic compound can be, for example vanadium(IV) oxo-bis(maltolato) (BMOV), ZnCl₂, bis(maltolato)zinc(II), zinc(II) complexes, vanadyl(IV) complexes, and the like. In certain embodiments, the culture medium comprises less than about 2 μg/ml of an insulin mimetic compound. In other embodiments, the culture medium comprises less than about 10 ng/ml, less than about 50 ng/ml, less than about 100 ng/ml, less than about 200 ng/ml, less than about 500 ng/ml, less than about 1 μg/ml, less than about 2 μg/ml, less than about 3 μg/ml, less than about 4 μg/ml, less than about 5 μg/ml, less than about 10 μg/ml, less than about 20 μg/ml, less than about 50 μg/ml, less than about 100 μg/ml or less than about 200 μg/ml of an insulin mimetic compound. Insulin mimetic compounds are known in the art and their synthesis, pharmacology, and activity have been described (Cocco et al., 2006; Sakurai and Adachi, 2005; Mehdi et al., 2006, each of which is hereby incorporated by reference in its entirety).

In a preferred embodiment of the present invention, hESCs are differentiated to ectoderm cells in a medium comprising less than about 2% serum, less than about 2 μg/ml insulin, less than about 2 μg/ml of an insulin analog, less than less than about 10 ng/ml of an insulin-like growth factor, less than 10 ng/ml of an insulin-like growth factor analog and/or less than 2 μg/ml of an insulin mimetic.

In some embodiments, the pluripotent cells are treated with an effective amount of an inhibitor of the PI-3-kinase pathway. Examples of PI-3-kinase pathway inhibitors include P-13-kinase antagonists, antagonists of the PI-3-kinase signal transduction cascade, compounds that decrease the synthesis or expression of endogenous PI-3-kinase, compounds that decrease release of endogenous PI-3-kinase, and compounds that inhibit activators of PI-3-kinase activity. In certain embodiments of the foregoing, the inhibitor is selected from the group consisting of Rapamycin, LY 294002, wortmannin, lithium chloride, Akt inhibitor I, Akt inhibitor II (SH-5), Akt inhibitor III (SH-6), NL-7′-101, and mixtures of the foregoing. Akt inhibitor I, II, Akt III, and NL-71-101 are commercially available from Calbiochem. In other embodiments, the inhibitor is selected from the group consisting of Rapamycin and LY 294002. In a further embodiment, the inhibitor comprises LY 294002.

In another embodiment, the inhibitor comprises Akt1-II. In other embodiments, the inhibitor is a molecule that inhibits an upstream component of the PI-3-kinase signaling pathway. In particular embodiments of the foregoing, the inhibitor is an inhibitor of an IGF or FGF receptor. It is understood that combinations of inhibitors may be used to elicit the desired effect.

In one embodiment, the inhibitor is Rapamycin. In certain embodiments, Rapamycin is initially present at a concentration of approximately 0.1 nM to approximately 500 nM, approximately 0.5 nM to approximately 250 nM, approximately 1.0 nM to approximately 150 nM, or approximately 1.5 nM to approximately 30 nM. In another embodiment, the inhibitor is LY 294002. In certain embodiments, LY 294002 is initially present at a concentration of approximately 1 μM to approximately 500 μM, approximately 2.5 μM to approximately 400 μM, approximately 5 μM to approximately 250 μM, approximately 10 μM to approximately 200 μM or approximately 20 μM to approximately 163 μM. In another embodiment, the inhibitor is Akt1-II. In certain embodiments, Akt1-II is initially present at a concentration of approximately 0.1 μM to approximately 500 μM, approximately 1 μM to approximately 250 μM, approximately 5 μM to approximately 20 μM, approximately 10 μM to approximately 100 μM or approximately 40 μM.

It will be appreciated that the aforementioned inhibitors of PI-3-kinase can be added to the cells under conditions where levels of serum, insulin, insulin analogs, insulin-like growth factors, insulin-like growth factor analogs or insulin-mimetic compounds are reduced or eliminated. In other words, in certain embodiments, inhibitors of PI-3-kinase can be added to a medium that lacks a substantial concentration or effective amount of one or more PI-3-kinase activators such as, serum, insulin, insulin analogs, insulin-like growth factors, insulin-like growth factor analogs or insulin-mimetic compounds. Alternatively, inhibitors of PI-3-kinase can be added to the cells under conditions where levels of serum, insulin, insulin analogs, insulin-like growth factors, insulin-like growth factor analogs, insulin-mimetic compounds have not been reduced or eliminated.

A cell differentiating medium or environment may be utilized to partially, terminally, or reversibly differentiate the pluripotent cells of the present invention, either prior to, during, or after contacting the pluripotent cells with at least one differentiation factor, and with a culture medium that limits PI-3-kinase signaling. In accordance with the invention the medium of the cell differentiation environment may contain a variety of components including, for example, KODMEM medium (Knockout Dulbecco's Modified Eagle's Medium), DMEM, F12 medium, FBS (fetal bovine serum), FGF2 (fibroblast growth factor 2), KSR or bLIF (human leukemia inhibitory factor). The cell differentiation environment can also contain supplements such as L-Glutamine, NEAA (non-essential amino acids), P/S (penicillin/streptomycin), N2 and β-mercaptoethanol (β-ME). It is contemplated that additional factors may be added to the cell differentiation environment, including, but not limited to, fibronectin, laminin, heparin, heparin sulfate, retinoic acid, members of the epidermal growth factor family (EGFs), members of the fibroblast growth factor family (FGFs) including FGF2 and/or FGF8, members of the platelet derived growth factor family (PDGFs), transforming growth factor (TGF)/bone morphogenetic protein (BMP)/growth and differentiation factor (GDF) factor family antagonists including but not limited to noggin, follistatin, chordin, gremlin, cerberus/DAN family proteins, ventropin, high dose activin, and amnionless. TGF/BMP/GDF antagonists could also be added in the form of TGF/BMP/GDF receptor-Fc chimeras. Other factors that may be added include molecules that can activate or inactivate signaling through Notch receptor family, including but not limited to proteins of the Delta-like and Jagged families as well as inhibitors of Notch processing or cleavage. Other growth factors may include members of the insulin like growth factor family (IGF), insulin, the wingless related (WNT) factor family, and the hedgehog factor family. Additional factors may be added to promote ectoderm stem/progenitor proliferation and survival as well as survival and differentiation of derivatives of these progenitors.

In other embodiments, the cell differentiation environment comprises plating the cells in an adherent culture. As used herein, the terms “plated” and “plating” refer to any process that allows a cell to be grown in adherent culture. As used herein, the term “adherent culture” refers to a cell culture system whereby cells are cultured on a solid surface, which may in turn be coated with a solid substrate that may in turn be coated with another surface coat of a substrate, such as those listed below, or any other chemical or biological material that allows the cells to proliferate or be stabilized in culture. The cells may or may not tightly adhere to the solid surface or to the substrate. In one embodiment, the cells are plated on matrigel coated plates. The substrate for the adherent culture may comprise anyone or combination of polyornithine, laminin, poly-lysine, purified collagen, gelatin, extracellular matrix, fibronectin, tenascin, vitronectin, entactin, heparin sulfate proteoglycans, poly glycolytic acid (PGA), poly lactic acid (PLA), poly lactic-glycolic acid (PLGA) and feeder layers such as, but not limited to, primary fibroblasts or fibroblast cells lines. Furthermore, the substrate for the adherent culture may comprise the extracellular matrix laid down by a feeder layer, or laid down by the pluripotent human cell or cell culture.

### Monitoring the Production of Ectoderm From Pluripotent Cells

The progression of the hESC culture to ectoderm can be monitored by determining the expression of markers characteristic of ectoderm. In some embodiments, the expression of certain markers is determined by detecting the presence or absence of the marker. Alternatively, the expression of certain markers can determined by measuring the level at which the marker is present in the cells of the cell culture or cell population. In such embodiments, the measurement of marker expression can be qualitative or quantitative. One method of quantitating the expression markers that are produced by marker genes is through the use of quantitative PCR (Q-PCR). Methods of performing Q-PCR are well known in the art. Other methods which are known in the art can also be used to quantitate marker gene expression. For example, the expression of a marker gene product can be detected by using antibodies specific for the marker gene product of interest. In some embodiments of the present invention, the expression of marker genes characteristic of ectoderm as well as the lack of significant expression of marker genes characteristic of hESCs and other cell types is determined.

As described further in the Examples below, a reliable marker of ectoderm is the PAX6 gene. As such, the ectoderm cells produced by the methods described herein express the PAX6 marker, thereby producing the PAX6 gene product. Another reliable marker of ectoderm is the SOX1 gene. As such, the ectoderm cells produced by the methods described herein express the SOX1 marker, thereby producing the SOX1 gene product. Other markers of ectoderm are ZIC1, cytokeratin, FGF5, HOXB1, LHX5, MASH1, MEIS1 and OTX1. In some embodiments of the present invention, ectoderm cells express the PAX6 marker and/or the SOX1 marker at a level higher than that of the SOX17 marker, which is characteristic of definitive endoderm (see Table 2). Additionally, in some embodiments, expression of the PAX6 marker and/or the SOX1 marker is higher than the expression of the OCT4 marker, which is characteristic of hESCs. In other embodiments of the present invention, ectoderm cells express the PAX6 marker and/or the SOX1 marker at a level higher than that of the AFP, SPARC or Thrombomodulin (TM) markers.

It will be appreciated that PAX6 marker and/or the SOX1 marker expression is induced over a range of different levels in ectoderm cells depending on the differentiation conditions. As such, in some embodiments of the present invention, the expression of the PAX6 marker and/or the SOX1 marker in ectoderm cells or cell populations is at least about 2-fold higher to at least about 10,000-fold higher than the expression of the PAX6 marker and/or the SOX1 marker in non-ectoderm cells or cell populations, for example pluripotent stem cells. In other embodiments of the present invention, the expression of the PAX6 marker and/or the SOX1 marker in ectoderm cells or cell populations is at least about 4-fold higher, at least about 6-fold higher, at least about 8-fold higher, at least about 10-fold higher, at least about 15-fold higher, at least about 20-fold higher, at least about 40-fold higher, at least about 80-fold higher, at least about 100-fold higher, at least about 150-fold higher, at least about 200-fold higher, at least about 500-fold higher, at least about 750-fold higher, at least about 1000-fold higher, at least about 2500-fold higher, at least about 5000-fold higher, at least about 7500-fold higher or at least about 10,000-fold higher than the expression of the PAX6 marker and/or the SOX1 marker in non-ectoderm cells or cell populations, for example pluripotent stem cells. In some embodiments, the expression of the PAX6 marker and/or the SOX1 marker in ectoderm cells or cell populations is infinitely higher than the expression of the PAX6 marker and/or the SOX1 marker in non-ectoderm cells or cell populations, for example pluripotent stem cells.

It will be appreciated that in some embodiments of the present invention, the expression of markers selected from the group consisting of ZIC1, cytokeratin, FGF5, HOXB1, LHX5, MASH1, MEIS1 and OTX1 in ectoderm cells or cell populations is increased as compared to the expression of ZIC1, cytokeratin, FGF5, HOXB1, LHX5, MASH 1, MEIS1 and OTX1 in non-ectoderm cells or cell populations.

It will also be appreciated that there is a range of differences between the expression level of the PAX6 marker and/or the SOX1 marker and the expression levels of the OCT4, SPARC, AFP, TM and/or SOX7 markers in ectoderm cells. As such, in some embodiments of the present invention, the expression of the PAX6 marker and/or the SOX1 marker is at least about 2-fold higher to at least about 10,000-fold higher than the expression of OCT4, SPARC, AFP, TM and/or SOX7 markers. In other embodiments of the present invention, the expression of the PAX6 marker and/or the SOX1 marker is at least about 4-fold higher, at least about 6-fold higher, at least about 8-fold higher, at least about 10-fold higher, at least about 15-fold higher, at least about 20-fold higher, at least about 40-fold higher, at least about 80-fold higher, at least about 100-fold higher, at least about 150-fold higher, at least about 200-fold higher, at least about 500-fold higher, at least about 750-fold higher, at least about 1000-fold higher, at least about 2500-fold higher, at least about 5000-fold higher, at least about 7500-fold higher or at least about 10,000-fold higher than the expression of OCT4, SPARC, AFP, TM and/or SOX7 markers. In some embodiments, OCT4, SPARC, AFP, TM and/or SOX7 markers are not significantly (substantially) expressed in ectoderm cells.

### Compositions Comprising Ectoderm

Some aspects of the present invention relate to compositions, such as cell populations and cell cultures, that comprise both pluripotent cells, such as stem cells, and multipotent ectoderm cells that can differentiate into cells of the neural ectoderm or non-neural ectoderm. For example, using the methods described herein, compositions comprising mixtures of hESCs and ectoderm cells can be produced. In some embodiments, compositions comprising at least about 5 ectoderm cells for about every 95 pluripotent cells are produced. In other embodiments, compositions comprising at least about 95 ectoderm cells for about every 5 pluripotent cells are produced. Additionally, compositions comprising other ratios of ectoderm cells to pluripotent cells are contemplated. For example, compositions comprising at least about 1 ectoderm cell for about every 1,000,000 pluripotent cells, at least about 1 ectoderm cell for about every 100,000 pluripotent cells, at least about 1 ectoderm cell for about every 10,000 pluripotent cells, at least about 1 ectoderm cell for about every 1000 pluripotent cells, at least about 1 ectoderm cell for about every 500 pluripotent cells, at least about 1 ectoderm cell for about every 100 pluripotent cells, at least about 1 ectoderm cell for about every 10 pluripotent cells, at least about 1 ectoderm cell for about every 5 pluripotent cells, at least about 1 ectoderm cell for about every 2 pluripotent cells, at least about 2 ectoderm cells for about every 1 pluripotent cell, at least about 5 ectoderm cells for about every 1 pluripotent cell, at least about 10 ectoderm cells for about every 1 pluripotent cell, at least about 20 ectoderm cells for about every 1 pluripotent cell, at least about 50 ectoderm cells for about every 1 pluripotent cell, at least about 100 ectoderm cells for about every 1 pluripotent cell, at least about 1000 ectoderm cells for about every 1 pluripotent cell, at least about 10,000 ectoderm cells for about every 1 pluripotent cell, at least about 100,000 ectoderm cells for about every 1 pluripotent cell and at least about 1,000,000 ectoderm cells for about every 1 pluripotent cell are contemplated. In some embodiments of the present invention, the pluripotent cells are human embryonic stem cells. In certain embodiments the stem cells are derived from a morula, the inner cell mass of an embryo or the gonadal ridges of an embryo. In certain other embodiments, the pluripotent cells are derived from the gonadal or germ tissues of a multicellular structure that has developed past the embryonic stage. In other embodiments, the stem cells are derived from preimplantation embryos.

Some aspects of the present invention relate to cell cultures or cell populations comprising from at least about 5% ectoderm cells to at least about 95% ectoderm cells. In some embodiments the cell cultures or cell populations comprise mammalian cells. In preferred embodiments, the cell cultures or cell populations comprise human cells. For example, certain specific embodiments relate to cell cultures comprising human cells, wherein from at least about 5% to at least about 95% of the human cells are ectoderm cells. Other embodiments of the present invention relate to cell cultures comprising human cells, wherein at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, about 95%, or greater than 95% of the human cells are ectoderm cells. In embodiments where the cell cultures or cell populations comprise human feeder cells, the above percentages are calculated without respect to the human feeder cells in the cell cultures or cell populations.

Further embodiments of the present invention relate to compositions, such as cell cultures or cell populations, comprising human cells, such as human ectoderm cells, wherein the expression of the PAX6 marker and/or the SOX1 marker is greater than the expression of the OCT 4, SPARC, alpha-fetoprotein (AFP), Thrombomodulin (TM) and/or SOX7 marker in at least about 5% of the human cells. In other embodiments, the expression of the PAX6 marker and/or the SOX1 marker is greater than the expression of the OCT4, SPARC, AFP, TM and/or SOX7 marker in at least about 10% of the human cells, in at least about 15% of the human cells, in at least about 20% of the human cells, in at least about 25% of the human cells, in at least about 30% of the human cells, in at least about 35% of the human cells, in at least about 40% of the human cells, in at least about 45% of the human cells, in at least about 50% of the human cells, in at least about 55% of the human cells, in at least about 60% of the human cells, in at least about 65% of the human cells, in at least about 70% of the human cells, in at least about 75% of the human cells, in at least about 80% of the human cells, in at least about 85% of the human cells, in at least about 90% of the human cells, in at least about 95% of the human cells or in greater than 95% of the human cells. In embodiments where the cell cultures or cell populations comprise human feeder cells, the above percentages are calculated without respect to the human feeder cells in the cell cultures or cell populations.

It will be appreciated that some embodiments of the present invention relate to compositions, such as cell cultures or cell populations, comprising human cells, such as human ectoderm cells, wherein the expression of one or more markers selected from the group consisting of ZIC1, cytokeratin, FGF5, HOXB1, LHX5, MASH1, MEIS1 and OTX1 is greater than the expression of the OCT4, SPARC, AFP, TM and/or SOX7 markers in from at least about 5% to greater than at least about 95% of the human cells. In embodiments where the cell cultures or cell populations comprise human feeder cells, the above percentages are calculated without respect to the human feeder cells in the cell cultures or cell populations.

It will be appreciated that some embodiments of the present invention relate to compositions, such as cell cultures or cell populations, comprising human cells, such as human ectoderm cells, wherein the expression of the ZIC1, cytokeratin, FGF5, HOXB1, LHX5, MASH1, MEIS1 and OTX1 markers is greater than the expression of the SOX17, OCT4, SPARC, AFP, TM and/or SOX7 markers in from at least about 5% to greater than at least about 95% of the human cells. In embodiments where the cell cultures or cell populations comprise human feeder cells, the above percentages are calculated without respect to the human feeder cells in the cell cultures or cell populations.

It will be appreciated that some embodiments of the present invention relate to compositions, such as cell cultures or cell populations, comprising human cells, such as human ectoderm cells, wherein the ectoderm cells do not substantially express SOX17.

Using the methods described herein, compositions comprising ectoderm cells substantially free of other cell types can be produced. In some embodiments of the present invention, the ectoderm cell populations or cell cultures produced by the methods described herein are substantially free of cells that significantly express the SOX17, OCT4, SOX7, AFP, SPARC and/or TM markers.

Further embodiments of the present invention relate to compositions, such as cell cultures or cell populations comprising human cells, further comprising a culture medium which comprises less than about 10% serum and lacks serum replacement. In certain embodiments of the present invention, serum concentrations can range from about 0.01% v/v to about 10% v/v. For example, in certain embodiments, the serum concentration of the medium can be less than about 0.01% (v/v), less than about 0.05% (v/v), less than about 0.1% (v/v), less than about 0.2% (v/v), less than about 0.3% (v/v), less than about 0.4% (v/v), less than about 0.5% (v/v), less than about 0.6% (v/v), less than about 0.7% (v/v), less than about 0.8% (v/v), less than about 0.9% (v/v), less than about 1% (v/v), less than about 2% (v/v), less than about 3% (v/v), less than about 4% (v/v), less than about 5% (v/v), less than about 6% (v/v), less than about 7% (v/v), less than about 8% (v/v), less than about 9% (v/v) or less than about 10% (v/v). In certain embodiments, the culture medium lacks serum and lacks serum replacement.

Further embodiments of the present invention relate to compositions, such as cell cultures or cell populations comprising human ectoderm cells, further comprising a culture medium which comprises less than about 2 μg/ml insulin. In other embodiments, the culture medium comprises less than about 10 ng/ml, less than about 50 ng/ml, less than about 100 ng/ml, less than about 200 ng/ml, less than about 500 ng/ml, less than about 1 μg/ml, less than about 2 μg/ml, less than about 3 μg/ml, less than about 4 μg/ml, less than about 5 μg/ml, less than about 10 μg/ml, less than about 20 μg/ml, less than about 50 μg/ml, less than about 100 μg/ml or less than about 200 μg/ml insulin. In certain embodiments, the culture medium comprises less than about 2 μg/ml of an insulin analog. In other embodiments, the culture medium comprises less than about 10 ng/ml, less than about 50 ng/ml, less than about 100 ng/ml, less than about 200 ng/ml, less than about 500 ng/ml, less than about 1 μg/ml, less than about 2 μg/ml, less than about 3 μg/ml, less than about 4 μg/ml, less than about 5 μg/ml, less than about 10 μg/ml, less than about 20 μg/ml, less than about 50 μg/ml, less than about 100 μg/ml or less than about 200 μg/ml of an insulin analog.

In certain embodiments, cell cultures or cell populations comprising human ectoderm cells comprise a culture medium that lacks a substantial concentration of an insulin-like growth factor or insulin-like growth factor analogs. The insulin-like growth factor can be, for example, insulin-like growth factor-1 (IGF-1), insulin-like growth factor-2 (IGF-2) or insulin-like growth factor analogs. In certain embodiments, the culture medium comprises less than about 10 ng/ml of insulin-like growth factor-1 or insulin-like growth factor analogs. In other embodiments, the culture medium comprises less than about 0.1 ng/ml, less than about 1 ng/ml, less than about 2 ng/ml, less than about 3 ng/ml, less than about 4 ng/ml, less than about 5 ng/ml, less than about 6 ng/ml, less than about 7 ng/ml, less than about 8 ng/ml, less than about 9 ng/ml, less than about 10 ng/ml, less than about 20 ng/ml, less than about 50 ng/ml, less than about 100 ng/ml or less than about 200 ng/ml of insulin-like growth factor or insulin-like growth factor analogs.

In other embodiments, cell cultures or cell populations comprising human ectoderm cells comprise a culture medium that lacks a substantial concentration of an insulin mimetic compound. The insulin mimetic compound can be, for example vanadium(IV) oxo-bis(maltolato) (BMOV), ZnCl₂, bis(maltolato)zinc(II), zinc(II) complexes, vanadyl(IV) complexes, and the like. In certain embodiments, the culture medium comprises less than about 2 μg/ml of an insulin mimetic compound. In other embodiments, the culture medium comprises less than about 10 ng/ml, less than about 50 ng/ml, less than about 100 ng/ml, less than about 200 ng/ml, less than about 500 ng/ml, less than about 1 μg/ml, less than about 2 μg/ml, less than about 3 μg/ml, less than about 4 μg/ml, less than about 5 μg/ml, less than about 10 μg/ml, less than about 20 μg/ml, less than about 50 μg/ml, less than about 100 μg/ml or less than about 200 μg/ml of an insulin mimetic compound.

### Production of Extraembryonic Endoderm From Pluripotent Cells

Human pluripotent cells are maintained in culture in a pluripotent state by routine passage until it is desired that they be differentiated into extraembryonic endoderm. In some embodiments, differentiation to extraembryonic endoderm is achieved by providing to the pluripotent cell culture a differentiation factor in an amount sufficient to promote differentiation to extraembryonic endoderm. In some embodiments, differentiation factors which are useful for the production of extraembryonic endoderm are selected from the BMP subgroup. In preferred embodiments of the differentiation methods described herein, the differentiation factor is BMP4. In certain embodiments of the present invention, a BMP differentiation factor in combination with one or more other differentiation factors can be used. In certain embodiments, the FGFR inhibitor SU5402 is provided alone or in combination with a BMP, such as BMP4, in order to further promote differentiation to the extraembryonic endoderm lineage.

With respect to some of the embodiments of differentiation methods described herein, one or more of the above-mentioned differentiation factors are provided to the cells so that the differentiation factors are present in the cultures at concentrations sufficient to promote differentiation of at least a portion of the human pluripotent cells to extraembryonic endoderm. In some embodiments of the present invention, the above-mentioned differentiation factors are present in the cell culture at a concentration of at least about 5 ng/ml, at least about 10 ng/ml, at least about 25 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, at least about 1000 ng/ml, at least about 2000 ng/ml, at least about 3000 ng/ml, at least about 4000 ng/ml, at least about 5000 ng/ml or more than about 5000 ng/ml. In certain embodiments, the FGFR inhibitor SU5402 is provided alone or in combination with a BMP differentiation factor and is present in the cell culture at a concentration of at least about 0.01 μM, at least about 0.1 μM, at least about 0.5 μM, at least about 1 μM, at least about 2 μM, at least about 5 μM, at least about 10 μM, at least about 20 μM, at least about 30 μM, at least about 40 μM, at least about 50 μM, at least about 100 μM, at least about 200 μM, at least about 500 μM or at least about 1 mM.

In certain embodiments of the present invention, the above-mentioned differentiation factors are removed from the cell culture subsequent to their addition. For example, the differentiation factors can be removed within about one day, about two days, about three days, about four days, about five days, about six days, about seven days, about eight days, about nine days or about ten days after their addition. In a preferred embodiment, the differentiation factors are removed from about three to about five days after their addition.

In some embodiments of the present invention, human pluripotent cells, such as hESCs, can be differentiated to extraembryonic endoderm cells containing reduced serum or no serum. The customary level of serum in culture medium for maintaining cell survival is 10% (v/v). Among other things, serum promotes the signaling of the PI-3-K pathway. It has been surprisingly discovered that PI-3-kinase signaling can restrict the potential of hESCs to differentiate to certain primary cell lineages. As such, in certain embodiments, the level of serum in the culture medium is reduced below the customary concentration in order to reduce PI-3-K signaling and promote differentiation. In some embodiments, the culture medium comprises less than about 10% (v/v) serum and lacks serum replacement. In certain embodiments of the present invention, serum concentrations can range from about 0.01% v/v to about 10% v/v. For example, in certain embodiments, the serum concentration of the medium can be less than about 0.01% (v/v), less than about 0.05% (v/v), less than about 0.1% (v/v), less than about 0.2% (v/v), less than about 0.3% (v/v), less than about 0.4% (v/v), less than about 0.5% (v/v), less than about 0.6% (v/v), less than about 0.7% (v/v), less than about 0.8% (v/v), less than about 0.9% (v/v), less than about 1% (v/v), less than about 2% (v/v), less than about 3% (v/v), less than about 4% (v/v), less than about 5% (v/v), less than about 6% (v/v), less than about 7% (v/v), less than about 8% (v/v), less than about 9% (v/v) and less than about 10% (v/v). In some embodiments, the hESCs are differentiated to extraembryonic endoderm cells without serum and without serum replacement. In still other embodiments, the hESCs are differentiated to extraembryonic endoderm cells in the presence of about 2% serum or less.

In some embodiments, the serum concentration is increased over time to promote survival and growth of the differentiating cells in culture. Thus, even though human pluripotent cells, such as hESCs, can be contacted with a culture medium containing reduced serum or no serum initially in order to limit PI-3-kinase signaling and promote differentiation to extraembryonic endoderm lineage, in certain embodiments the serum concentration is increased over time. In such embodiments, the serum concentration is increased after about 1 day from initially contacting the cells with culture medium containing reduced serum or no serum. In other embodiments, the serum concentration can be increased after about 0.5 days, about 1 day, about 2 days, about 3 days, about 4 days, about 5 days, or after about 6 days after the initial contacting step. After about 1 day after contacting the cells with culture medium containing reduced serum or no serum, the concentration of serum present in the culture medium can be about 0.01% (v/v), about 0.05% (v/v), about 0.1% (v/v), about 0.2% (v/v), about 0.3% (v/v), about 0.4% (v/v), about 0.5% (v/v), about 0.6% (v/v), about 0.7% (v/v), about 0.8% (v/v), about 0.9% (v/v), about 1% (v/v), about 2% (v/v), about 3% (v/v), about 4% (v/v), about 5% (v/v), about 6% (v/v), about 7% (v/v), about 8% (v/v), about 9% (v/v), about 10% (v/v), about 15% (v/v) or about 20% (v/v). After about 2 days after contacting the cells with culture medium containing reduced serum or no serum, the concentration of serum present in the culture medium can be about 0.01% (v/v), about 0.05% (v/v), about 0.1% (v/v), about 0.2% (v/v), about 0.3% (v/v), about 0.4% (v/v), about 0.5% (v/v), about 0.6% (v/v), about 0.7% (v/v), about 0.8% (v/v), about 0.9% (v/v), about 1% (v/v), about 2% (v/v), about 3% (v/v), about 4% (v/v), about 5% (v/v), about 6% (v/v), about 7% (v/v), about 8% (v/v), about 9% (v/v), about 10% (v/v), about 15% (v/v) or about 20% (v/v).

In some embodiments of the present invention, cultures of human pluripotent cells, such as hESCs, can be differentiated to extraembryonic endoderm cells in medium that lacks a substantial concentration of a molecule that promotes PI-3-kinase signaling activity. Molecules that activate PI-3-kinase signaling activity are known in the art, and include, for example, insulin, insulin analogs, insulin-like growth factors, insulin-like growth factor analogs, insulin-mimetic compounds and combinations thereof. In certain embodiments, the culture medium comprises less than about 2 μg/ml insulin. In other embodiments, the culture medium comprises less than about 10 ng/ml, less than about 50 ng/ml, less than about 100 ng/ml, less than about 200 ng/ml, less than about 500 ng/ml, less than about 1 μg/ml, less than about 2 μg/ml, less than about 3 μg/ml, less than about 4 μg/ml, less than about 5 μg/ml, less than about 10 μg/ml, less than about 20 μg/ml, less than about 50 μg/ml, less than about 100 μg/ml or less than about 200 μg/ml insulin. In certain embodiments, the culture medium comprises less than about 2 μg/ml of an insulin analog. In other embodiments, the culture medium comprises less than about 10 ng/ml, less than about 50 ng/ml, less than about 100 ng/ml, less than about 200 ng/ml, less than about 500 ng/ml, less than about 1 μg/ml, less than about 2 μg/ml, less than about 3 μg/ml, less than about 4 μg/ml, less than about 5 μg/ml, less than about 10 μg/ml, less than about 20 μg/ml, less than about 50 μg/ml, less than about 100 μg/ml or less than about 200 μg/ml of an insulin analog.

In certain embodiments of the present invention, the culture medium lacks a substantial concentration of an insulin-like growth factor or insulin-like growth factor analogs. The insulin-like growth factor can be, for example, insulin-like growth factor-1 (IGF-1), insulin-like growth factor-2 (IGF-2) or any other insulin-like growth factor analogs. In certain embodiments, the culture medium comprises less than about 10 ng/ml of insulin-like growth factor-1 or insulin-like growth factor analogs. In other embodiments, the culture medium comprises less than about 0.1 ng/ml, less than about 1 ng/ml, less than about 2 ng/ml, less than about 3 ng/ml, less than about 4 ng/ml, less than about 5 ng/ml, less than about 6 ng/ml, less than about 7 ng/ml, less than about 8 ng/ml, less than about 9 ng/ml, less than about 10 ng/ml, less than about 20 ng/ml, less than about 50 ng/ml, less than about 100 ng/ml or less than about 200 ng/ml of insulin-like growth factor or insulin-like growth factor analogs.

In certain embodiments, the culture medium lacks a substantial concentration of an insulin mimetic compound. The insulin mimetic compound can be, for example vanadium(IV) oxo-bis(maltolato) (BMOV), ZnCl₂, bis(maltolato)zinc(II), zinc(II) complexes, vanadyl(IV) complexes, and the like. In certain embodiments, the culture medium comprises less than about 2 μg/ml of an insulin mimetic compound. In other embodiments, the culture medium comprises less than about 10 ng/ml, less than about 50 ng/ml, less than about 100 ng/ml, less than about 200 ng/ml, less than about 500 ng/ml, less than about 1 μg/ml, less than about 2 μg/ml, less than about 3 μg/ml, less than about 4 μg/ml, less than about 5 μg/ml, less than about 10 less than about 20 μg/ml, less than about 50 μg/ml, less than about 100 ng/ml or less than about 200 μg/ml of an insulin mimetic compound. Insulin mimetic compounds are known in the art and their synthesis, pharmacology, and activity have been described (Cocco et al., 2006; Sakurai and Adachi, 2005; Mehdi et al., 2006, each of which is hereby incorporated by reference in its entirety).

In a preferred embodiment of the present invention, hESCs are differentiated to extraembryonic endoderm cells in a medium comprising less than about 2% serum, less than about 2 μg/ml insulin, less than about 2 μg/ml of an insulin analog, less than less than about 10 ng/ml of an insulin-like growth factor, less than 10 ng/ml of an insulin-like growth factor analog and/or less than 2 μg/ml of an insulin mimetic.

In some embodiments, the pluripotent cells are treated with an effective amount of an inhibitor of the PI-3-kinase pathway. Examples of PI-3-kinase pathway inhibitors include P-13-kinase antagonists, antagonists of the PI-3-kinase signal transduction cascade, compounds that decrease the synthesis or expression of endogenous PI-3-kinase, compounds that decrease release of endogenous PI-3-kinase, and compounds that inhibit activators of PI-3-kinase activity. In certain embodiments of the foregoing, the inhibitor is selected from the group consisting of Rapamycin, LY 294002, wortmannin, lithium chloride, Akt inhibitor I, Akt inhibitor II (SH-5), Akt inhibitor III (SH-6), NL-71-101, and mixtures of the foregoing. Akt inhibitor I, II, Akt III, and NL-71-101 are commercially available from Calbiochem. In other embodiments, the inhibitor is selected from the group consisting of Rapamycin and LY 294002. In a further embodiment, the inhibitor comprises LY 294002. In another embodiment, the inhibitor comprises Akt1-II. In other embodiments, the inhibitor is a molecule that inhibits an upstream component of the PI-3-kinase signaling pathway. In particular embodiments of the foregoing, the inhibitor is an inhibitor of an IGF or FGF receptor. It is understood that combinations of inhibitors may be used to elicit the desired effect.

In one embodiment, the inhibitor is Rapamycin. In certain embodiments, Rapamycin is initially present at a concentration of approximately 0.1 nM to approximately 500 nM, approximately 0.5 nM to approximately 250 nM, approximately 1.0 nM to approximately 150 nM, or approximately 1.5 nM to approximately 30 nM. In another embodiment, the inhibitor is LY 294002. In certain embodiments, LY 294002 is initially present at a concentration of approximately 1 μM to approximately 500 μM, approximately 2.5 μM to approximately 400 μM, approximately 5 μM to approximately 250 μM, approximately 10 μM to approximately 200 μM or approximately 20 μM to approximately 163 μM. In another embodiment, the inhibitor is Akt1-II. In certain embodiments, Akt1-II is initially present at a concentration of approximately 0.1 μM to approximately 500 μM, approximately 1 μM to approximately 250 μM, approximately 5 μM to approximately 20 μM, approximately 10 μM to approximately 100 μM or approximately 40 μM.

It will be appreciated that the aforementioned inhibitors of PI-3-kinase can be added to the cells under conditions where levels of serum, insulin, insulin analogs, insulin-like growth factors, insulin-like growth factor analogs or insulin-mimetic compounds are reduced or eliminated. In other words, in certain embodiments, inhibitors of PI-3-kinase can be added to a medium that lacks a substantial concentration or effective amount of one or more PI-3-kinase activators such as, serum, insulin, insulin analogs, insulin-like growth factors, insulin-like growth factor analogs or insulin-mimetic compounds. Alternatively, inhibitors of PI-3-kinase can be added to the cells under conditions where levels of serum, insulin, insulin analogs, insulin-like growth factors, insulin-like growth factor analogs, insulin-mimetic compounds have not been reduced or eliminated.

A cell differentiating medium or environment may be utilized to partially, terminally, or reversibly differentiate the pluripotent cells of the present invention, either prior to, during, or after contacting the pluripotent cells with at least one differentiation factor, and with a culture medium that limits PI-3-kinase signaling. In accordance with the invention the medium of the cell differentiation environment may contain a variety of components including, for example, KODMEM medium (Knockout Dulbecco's Modified Eagle's Medium), DMEM, Ham's F12 medium, FBS (fetal bovine serum), FGF2 (fibroblast growth factor 2), KSR or bLIF (human leukemia inhibitory factor). The cell differentiation environment can also contain supplements such as L-Glutamine, NEAA (non-essential amino acids), P/S (penicillin/streptomycin), N2 and β-mercaptoethanol (β-ME). It is contemplated that additional factors may be added to the cell differentiation environment, including, but not limited to, fibronectin, laminin, heparin, heparin sulfate, retinoic acid, members of the epidermal growth factor family (EGFs), members of the fibroblast growth factor family (FGFs) including FGF2 and/or FGF8, members of the platelet derived growth factor family' (PDGFs), transforming growth factor (TGF)/bone morphogenetic protein (BMP)/growth and differentiation factor (GDF) factor family antagonists including but not limited to noggin, follistatin, chordin, gremlin, cerberus/DAN family proteins, ventropin, high dose activin, and amnionless. TGF/BMP/GDF antagonists could also be added in the form of TGF/BMP/GDF receptor-Fc chimeras. Other factors that may be added include molecules that can activate or inactivate signaling through Notch receptor family, including but not limited to proteins of the Delta-like and Jagged families as well as inhibitors of Notch processing or cleavage. Other growth factors may include members of the insulin like growth factor family (IGF), insulin, the wingless related (WNT) factor family, and the hedgehog factor family. Additional factors may be added to promote extraembryonic endoderm stem/progenitor proliferation and survival as well as survival and differentiation of derivatives of these progenitors.

In other embodiments, the cell differentiation environment comprises plating the cells in an adherent culture. As used herein, the terms “plated” and “plating” refer to any process that allows a cell to be grown in adherent culture. As used herein, the term “adherent culture” refers to a cell culture system whereby cells are cultured on a solid surface, which may in turn be coated with a solid substrate that may in turn be coated with another surface coat of a substrate, such as those listed below, or any other chemical or biological material that allows the cells to proliferate or be stabilized in culture. The cells may or may not tightly adhere to the solid surface or to the substrate. In one embodiment, the cells are plated on matrigel coated plates. The substrate for the adherent culture may comprise anyone or combination of polyornithine, laminin, poly-lysine, purified collagen, gelatin, extracellular matrix, fibronectin, tenascin, vitronectin, entactin, heparin sulfate proteoglycans, poly glycolytic acid (PGA), poly lactic acid (PLA), poly lactic-glycolic acid (PLGA) and feeder layers such as, but not limited to, primary fibroblasts or fibroblast cells lines. Furthermore, the substrate for the adherent culture may comprise the extracellular matrix laid down by a feeder layer, or laid down by the pluripotent human cell or cell culture.

### Monitoring the Production of Extraembryonic Endoderm From Pluripotent Cells

The progression of the hESC culture to extraembryonic endoderm can be monitored by determining the expression of markers characteristic of extraembryonic endoderm. In some embodiments, the expression of certain markers is determined by detecting the presence or absence of the marker. Alternatively, the expression of certain markers can determined by measuring the level at which the marker is present in the cells of the cell culture or cell population. In such embodiments, the measurement of marker expression can be qualitative or quantitative. One method of quantitating the expression markers that are produced by marker genes is through the use of quantitative PCR (Q-PCR). Methods of performing Q-PCR are well known in the art. Other methods which are known in the art can also be used to quantitate marker gene expression. For example, the expression of a marker gene product can be detected by using antibodies specific for the marker gene product of interest. In some embodiments of the present invention, the expression of marker genes characteristic of extraembryonic endoderm as well as the lack of significant expression of marker genes characteristic of hESCs and other cell types is determined.

As described further in the Examples below, a reliable marker of extraembryonic endoderm is the SOX7 gene. As such, the extraembryonic endoderm cells produced by the methods described herein express the SOX7 marker, thereby producing the SOX7 gene product. Other markers of extraembryonic endoderm are alpha-fetoprotein (AFP), SPARC and Thrombomodulin (TM). In some embodiments of the present invention, extraembryonic endoderm cells express the SOX7 marker at a level higher than that of the SOX17 or CXCR4 marker, each of which is characteristic of definitive endoderm. Additionally, in some embodiments, expression of the SOX7 marker is higher than the expression of the OCT4 marker, which is characteristic of hESCs. In other embodiments of the present invention, extraembryonic endoderm cells express the SOX7 marker at a level higher than that of the SOX17, CXCR4 or OCT4 markers.

It will be appreciated that SOX7 marker expression is induced over a range of different levels in extraembryonic endoderm cells depending on the differentiation conditions. As such, in some embodiments of the present invention, the expression of the SOX7 marker in extraembryonic endoderm cells or cell populations is at least about 2-fold higher to at least about 10,000-fold higher than the expression of the SOX7 marker in non-extraembryonic endoderm cells or cell populations, for example pluripotent stem cells. In other embodiments of the present invention, the expression of the SOX7 marker in extraembryonic endoderm cells or cell populations is at least about 4-fold higher, at least about 6-fold higher, at least about 8-fold higher, at least about 10-fold higher, at least about 15-fold higher, at least about 20-fold higher, at least about 40-fold higher, at least about 80-fold higher, at least about 100-fold higher, at least about 150-fold higher, at least about 200-fold higher, at least about 500-fold higher, at least about 750-fold higher, at least about 1000-fold higher, at least about 2500-fold higher, at least about 5000-fold higher, at least about 7500-fold higher or at least about 10,000-fold higher than the expression of the SOX7 marker in non-extraembryonic endoderm cells or cell populations, for example pluripotent stem cells. In some embodiments, the expression of the SOX7 marker in extraembryonic endoderm cells or cell populations is infinitely higher than the expression of the SOX7 marker in non-extraembryonic endoderm cells or cell populations, for example pluripotent stem cells.

It will be appreciated that in some embodiments of the present invention, the expression of markers selected from the group consisting of SOX7, alpha-fetoprotein (AFP), SPARC and Thrombomodulin (TM) in extraembryonic endoderm cells or cell populations is increased as compared to the expression of SOX7, alpha-fetoprotein (AFP), SPARC and Thrombomodulin (TM) in non-extraembryonic endoderm cells or cell populations.

It will also be appreciated that there is a range of differences between the expression level of the SOX7 marker and the expression levels of the SOX17, CXCR4 or OCT4 markers in extraembryonic endoderm cells. As such, in some embodiments of the present invention, the expression of the SOX7 marker is at least about 2-fold higher to at least about 10,000-fold higher than the expression of SOX17, CXCR4 or OCT4 markers. In other embodiments of the present invention, the expression of the SOX7 marker is at least about 4-fold higher, at least about 6-fold higher, at least about 8-fold higher, at least about 10-fold higher, at least about 15-fold higher, at least about 20-fold higher, at least about 40-fold higher, at least about 80-fold higher, at least about 100-fold higher, at least about 150-fold higher, at least about 200-fold higher, at least about 500-fold higher, at least about 750-fold higher, at least about 1000-fold higher, at least about 2500-fold higher, at least about 5000-fold higher, at least about 7500-fold higher or at least about 10,000-fold higher than the expression of the SOX17, CXCR4 or OCT4 markers. In some embodiments, the CXCR4 or OCT4 markers are not significantly (substantially) expressed in extraembryonic endoderm cells.

### Compositions Comprising Extraembryonic Endoderm

Some aspects of the present invention relate to compositions, such as cell populations and cell cultures, that comprise both pluripotent cells, such as stem cells, and multipotent extraembryonic endoderm cells that can differentiate into cells of the visceral endoderm or parietal endoderm. For example, using the methods described herein, compositions comprising mixtures of hESCs and extraembryonic endoderm cells can be produced. In some embodiments, compositions comprising at least about 5 extraembryonic endoderm cells for about every 95 pluripotent cells are produced. In other embodiments, compositions comprising at least about 95 extraembryonic endoderm cells for about every 5 pluripotent cells are produced. Additionally, compositions comprising other ratios of extraembryonic endoderm cells to pluripotent cells are contemplated. For example, compositions comprising at least about 1 extraembryonic endoderm cell for about every 1,000,000 pluripotent cells, at least about 1 extraembryonic endoderm cell for about every 100,000 pluripotent cells, at least about 1 extraembryonic endoderm cell for about every 10,000 pluripotent cells, at least about 1 extraembryonic endoderm cell for about every 1000 pluripotent cells, at least about 1 extraembryonic endoderm cell for about every 500 pluripotent cells, at least about 1 extraembryonic endoderm cell for about every 100 pluripotent cells, at least about 1 extraembryonic endoderm cell for about every 10 pluripotent cells, at least about 1 extraembryonic endoderm cell for about every 5 pluripotent cells, at least about 1 extraembryonic endoderm cell for about every 2 pluripotent cells, at least about 2 extraembryonic endoderm cells for about every 1 pluripotent cell, at least about 5 extraembryonic endoderm cells for about every 1 pluripotent cell, at least about 10 extraembryonic endoderm cells for about every 1 pluripotent cell, at least about 20 extraembryonic endoderm cells for about every 1 pluripotent cell, at least about 50 extraembryonic endoderm cells for about every 1 pluripotent cell, at least about 100 extraembryonic endoderm cells for about every 1 pluripotent cell, at least about 1000 extraembryonic endoderm cells for about every 1 pluripotent cell, at least about 10,000 extraembryonic endoderm cells for about every 1 pluripotent cell, at least about 100,000 extraembryonic endoderm cells for about every 1 pluripotent cell and at least about 1,000,000 extraembryonic endoderm cells for about every 1 pluripotent cell are contemplated. In some embodiments of the present invention, the pluripotent cells are human embryonic stem cells. In certain embodiments the stem cells are derived from a morula, the inner cell mass of an embryo or the gonadal ridges of an embryo. In certain other embodiments, the pluripotent cells are derived from the gonadal or germ tissues of a multicellular structure that has developed past the embryonic stage. In other embodiments, the stem cells are derived from preimplantation embryos.

Some aspects of the present invention relate to cell cultures or cell populations comprising from at least about 5% extraembryonic endoderm cells to at least about 95% extraembryonic endoderm cells. In some embodiments the cell cultures or cell populations comprise mammalian cells. In preferred embodiments, the cell cultures or cell populations comprise human cells. For example, certain specific embodiments relate to cell cultures comprising human cells, wherein from at least about 5% to at least about 95% of the human cells are extraembryonic endoderm cells. Other embodiments of the present invention relate to cell cultures comprising human cells, wherein at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, about 95%, or greater than 95% of the human cells are extraembryonic endoderm cells. In embodiments where the cell cultures or cell populations comprise human feeder cells, the above percentages are calculated without respect to the human feeder cells in the cell cultures or cell populations.

Further embodiments of the present invention relate to compositions, such as cell cultures or cell populations, comprising human cells, such as human extraembryonic endoderm cells, wherein the expression of the SOX7 marker is greater than the expression of the SOX17, CXCR4 or OCT4 marker in at least about 5% of the human cells. In other embodiments, the expression of the SOX7 marker is greater than the expression of the SOX17, CXCR4 or OCT4 marker in at least about 10% of the human cells, in at least about 15% of the human cells, in at least about 20% of the human cells, in at least about 25% of the human cells, in at least about 30% of the human cells, in at least about 35% of the human cells, in at least about 40% of the human cells, in at least about 45% of the human cells, in at least about 50% of the human cells, in at least about 55% of the human cells, in at least about 60% of the human cells, in at least about 65% of the human cells, in at, least about 70% of the human cells, in at least about 75% of the human cells, in at least about 80% of the human cells, in at least about 85% of the human cells, in at least about 90% of the human cells, in at least about 95% of the human cells or in greater than 95% of the human cells. In embodiments where the cell cultures or cell populations comprise human feeder cells, the above percentages are calculated without respect to the human feeder cells in the cell cultures or cell populations.

It will be appreciated that some embodiments of the present invention relate to compositions, such as cell cultures or cell populations, comprising human cells, such as human extraembryonic endoderm cells, wherein the expression of one or more markers selected from the group consisting of SOX7, alpha-fetoprotein (AFP), SPARC and Thrombomodulin (TM) is greater than the expression of the SOX17, CXCR4 or OCT4 markers in from at least about 5% to greater than at least about 95% of the human cells. In embodiments where the cell cultures or cell populations comprise human feeder cells, the above percentages are calculated without respect to the human feeder cells in the cell cultures or cell populations.

It will be appreciated that some embodiments of the present invention relate to compositions, such as cell cultures or cell populations, comprising human cells, such as human extraembryonic endoderm cells, wherein the expression of the SOX7, alpha-fetoprotein (AFP), SPARC and Thrombomodulin (TM) markers is greater than the expression of the SOX17, CXCR4 or OCT4 markers in from at least about 5% to greater than at least about 95% of the human cells. In embodiments where the cell cultures or cell populations comprise human feeder cells, the above percentages are calculated without respect to the human feeder cells in the cell cultures or cell populations.

It will be appreciated that some embodiments of the present invention relate to compositions, such as cell cultures or cell populations, comprising human cells, such as human extraembryonic endoderm cells, wherein the extraembryonic endoderm cells do not substantially express CXCR4.

Using the methods described herein, compositions comprising extraembryonic endoderm cells substantially free of other cell types can be produced. In some embodiments of the present invention, the extraembryonic endoderm cell populations or cell cultures produced by the methods described herein are substantially free of cells that significantly express the CXCR4 and/or OCT4 markers.

Further embodiments of the present invention relate to compositions, such as cell cultures or cell populations comprising human cells, further comprising a culture medium which comprises less than about 10% serum and lacks serum replacement. In certain embodiments of the present invention, serum concentrations can range from about 0.01% v/v to about 10% v/v. For example, in certain embodiments, the serum concentration of the medium can be less than about 0.01% (v/v), less than about 0.05% (v/v), less than about 0.1% (v/v), less than about 0.2% (v/v), less than about 0.3% (v/v), less than about 0.4% (v/v), less than about 0.5% (v/v), less than about 0.6% (v/v), less than about 0.7% (v/v), less than about 0.8% (v/v), less than about 0.9% (v/v), less than about 1% (v/v), less than about 2% (v/v), less than about 3% (v/v), less than about 4% (v/v), less than about 5% (v/v), less than about 6% (v/v), less than about 7% (v/v), less than about 8% (v/v), less than about 9% (v/v) or less than about 10% (v/v). In certain embodiments, the culture medium lacks serum and lacks serum replacement.

Further embodiments of the present invention relate to compositions, such as cell cultures or cell populations comprising human extraembryonic endoderm cells, further comprising a culture medium which comprises less than about 2 μg/ml insulin. In other embodiments, the culture medium comprises less than about 10 ng/ml, less than about 50 ng/ml, less than about 100 ng/ml, less than about 200 ng/ml, less than about 500 ng/ml, less than about 1 μg/ml, less than about 2 μg/ml, less than about 3 μg/ml, less than about 4 μg/ml, less than about 5 μg/ml, less than about 10 μg/ml, less than about 20 μg/ml, less than about 50 μg/ml, less than about 100 μg/ml or less than about 200 μg/ml insulin. In certain embodiments, the culture medium comprises less than about 2 μg/ml of an insulin analog. In other embodiments, the culture medium comprises less than about 10 ng/ml, less than about 50 ng/ml, less than about 100 ng/ml, less than about 200 ng/ml, less than about 500 ng/ml, less than about 1 μg/ml, less than about 2 μg/ml, less than about 3 μg/ml, less than about 4 μg/ml, less than about 5 μg/ml, less than about 10 μg/ml, less than about 20 μg/ml, less than about 50 μg/ml, less than about 100 μg/ml or less than about 200 μg/ml of an insulin analog.

In certain embodiments, cell cultures or cell populations comprising human extraembryonic endoderm cells comprise a culture medium that lacks a substantial concentration of an insulin-like growth factor or insulin-like growth factor analogs. The insulin-like growth factor can be, for example, insulin-like growth factor-1 (IGF-1), insulin-like growth factor-2 (IGF-2) or insulin-like growth factor analogs. In certain embodiments, the culture medium comprises less than about 10 ng/ml of insulin-like growth factor-1 or insulin-like growth factor analogs. In other embodiments, the culture medium comprises less than about 0.1 ng/ml, less than about 1 ng/ml, less than about 2 ng/ml, less than about 3 ng/ml, less than about 4 ng/ml, less than about 5 ng/ml, less than about 6 ng/ml, less than about 7 ng/ml, less than about 8 ng/ml, less than about 9 ng/ml, less than about 10 ng/ml, less than about 20 ng/ml, less than about 50 ng/ml, less than about 100 ng/ml or less than about 200 ng/ml of insulin-like growth factor or insulin-like growth factor analogs.

In other embodiments, cell cultures or cell populations comprising human extraembryonic endoderm cells comprise a culture medium that lacks a substantial concentration of an insulin mimetic compound. The insulin mimetic compound can be, for example vanadium(IV) oxo-bis(maltolato) (BMOV), ZnCl₂, bis(maltolato)zinc(II), zinc(II) complexes, vanadyl(IV) complexes, and the like. In certain embodiments, the culture medium comprises less than about 2 μg/ml of an insulin mimetic compound. In other embodiments, the culture medium comprises less than about 10 ng/ml, less than about 50 ng/ml, less than about 100 ng/ml, less than about 200 ng/ml, less than about 500 ng/ml, less than about 1 ng/ml, less than about 2 μg/ml, less than about 3 μg/ml, less than about 4 μg/ml, less than about 5 μg/ml, less than about 10 μg/ml, less than about 20 μg/ml, less than about 50 μg/ml, less than about 100 μg/ml or less than about 200 μg/ml of an insulin mimetic compound.

Enrichment, Isolation and/or Purification of Trophectoderm, Ectoderm and/or Extraembryonic Endoderm Cells

With respect to additional aspects of the processes described herein, trophectoderm, ectoderm and/or extraembryonic endoderm cells can be enriched, isolated and/or purified. In some embodiments, cell populations enriched for trophectoderm, ectoderm and/or extraembryonic endoderm cells are produced by isolating such cells from cell cultures.

In some embodiments of the processes described herein, trophectoderm, ectoderm and/or extraembryonic endoderm cells are fluorescently labeled then isolated from non-labeled cells by using a fluorescence activated cell sorter (FACS). In such embodiments, a nucleic acid encoding a fluorescent protein, such as enhanced green fluorescent protein (EGFP) green fluorescent protein (GFP), luciferase or another nucleic acid encoding an expressible fluorescent marker, is used to label trophectoderm, ectoderm and/or extraembryonic endoderm cells. For example, in some embodiments, at least one copy of a nucleic acid encoding EGFP or a biologically active fragment thereof is introduced into a pluripotent cell, preferably a human embryonic stem cell, downstream of the CDX2 (trophectoderm), SOX1 (ectoderm) or SOX7 (extraembryonic endoderm) promoter such that the expression of the EGFP gene product or biologically active fragment thereof is under control of the CDX2, SOX1 or SOX7 promoter. In some embodiments, the entire coding region of the nucleic acid, which encodes CDX2, SOX1 or SOX7, is replaced by a nucleic acid encoding EGFP or a biologically active fragment thereof. In other embodiments, the nucleic acid encoding EGFP or a biologically active fragment thereof is fused in frame with at least a portion of the nucleic acid encoding CDX2, SOX1 or SOX7, thereby generating a fusion protein. In such embodiments, the fusion protein retains a fluorescent activity similar to EGFP.

Fluorescently marked cells, such as the above-described pluripotent cells, are differentiated to trophectoderm, ectoderm and/or extraembryonic endoderm cells as described previously above. Because trophectoderm, ectoderm and/or extraembryonic endoderm cells express the fluorescent marker, whereas the other cell types present in the culture do not, the fluorescent cells can be separated from the non-fluorescent cells. In some embodiments, cell suspensions comprising a mixture of fluorescently-labeled trophectoderm, ectoderm and/or extraembryonic endoderm cells and unlabeled cell types are sorted using a FACS. Trophectoderm, ectoderm and/or extraembryonic endoderm cells are collected separately from unlabeled cells, thereby resulting in the isolation of such cell types. If desired, the isolated cell compositions can be further purified by additional rounds of sorting using the same or different markers that are specific for trophectoderm, ectoderm and/or extraembryonic endoderm cells.

In addition to the procedures just described, trophectoderm, ectoderm and/or extraembryonic endoderm cells may also be isolated by other techniques for cell isolation. Additionally, trophectoderm, ectoderm and/or extraembryonic endoderm cells may also be enriched or isolated by methods of serial subculture in growth conditions which promote the selective survival or selective expansion of said trophectoderm, ectoderm and/or extraembryonic endoderm cells.

It will be appreciated that the above-described enrichment, isolation and purification procedures can be used with such cultures at any stage of differentiation.

Using the methods described herein, enriched, isolated and/or purified populations of trophectoderm, ectoderm and/or extraembryonic endoderm cells can be produced in vitro from hESC cultures or populations which have undergone at least some differentiation. In some embodiments, the cells undergo random differentiation. In a preferred embodiment, however, the cells are directed to differentiate primarily into trophectoderm, ectoderm and/or extraembryonic endoderm cells. Some preferred enrichment, isolation and/or purification methods relate to the in vitro production of trophectoderm, ectoderm and/or extraembryonic endoderm cells from human embryonic stem cells.

Using the methods described herein, cell populations or cell cultures can be enriched in trophectoderm, ectoderm and/or extraembryonic endoderm cell content by at least about 2- to about 1000-fold as compared to untreated cell populations or cell cultures. In some embodiments, trophectoderm, ectoderm and/or extraembryonic endoderm cells can be enriched by at least about 5- to about 500-fold as compared to untreated cell populations or cell cultures. In other embodiments, trophectoderm, ectoderm and/or extraembryonic endoderm cells can be enriched from at least about 10- to about 200-fold as compared to untreated cell populations or cell cultures. In still other embodiments, trophectoderm, ectoderm and/or extraembryonic endoderm cells can be enriched from at least about 20- to about 100-fold as compared to untreated cell populations or cell cultures. In yet other embodiments, trophectoderm, ectoderm and/or extraembryonic endoderm cells can be enriched from at least about 40- to about 80-fold as compared to untreated cell populations or cell cultures. In certain embodiments, trophectoderm, ectoderm and/or extraembryonic endoderm cells can be enriched from at least about 2- to about 20-fold as compared to untreated cell populations or cell cultures.

### Identification of Factors Capable of Promoting the Differentiation of Trophectoderm, Ectoderm or Extraembryonic Endoderm Cells

Certain screening methods described herein relate to methods for identifying at least one differentiation factor that is capable of promoting the differentiation of trophectoderm, ectoderm and/or extraembryonic endoderm cells. In some embodiments of these methods, cell populations comprising human trophectoderm, ectoderm or extraembryonic endoderm cells are obtained. The cell population is then provided with a candidate differentiation factor. At a first time point, which is prior to or at approximately the same time as providing the candidate differentiation factor, expression of a marker is determined. Alternatively, expression of the marker can be determined after providing the candidate differentiation factor. At a second time point, which is subsequent to the first time point and subsequent to the step of providing the candidate differentiation factor to the cell population, expression of the same marker is again determined. Whether the candidate differentiation factor is capable of promoting the differentiation of the trophectoderm, ectoderm or extraembryonic endoderm cells is determined by comparing expression of the marker at the first time point with the expression of the marker at the second time point. If expression of the marker at the second time point is increased or decreased as compared to expression of the marker at the first time point, then the candidate differentiation factor is capable of promoting the differentiation of, trophectoderm, ectoderm or extraembryonic endoderm cells.

Some embodiments of the screening methods described herein utilize cell populations or cell cultures which comprise human trophectoderm, ectoderm or extraembryonic endoderm cells. For example, the cell population can be a substantially purified population of human trophectoderm, ectoderm or extraembryonic endoderm cells. Alternatively, the cell population can be an enriched population of human trophectoderm, ectoderm or extraembryonic endoderm cells, wherein at least about 90%, at least about 91%, at least about 92%, at least about 93%, at least about 94%, at least about 95%, at least about 96%, at least about 97% or greater than at least about 97% of the human cells in the cell population are human trophectoderm, ectoderm or extraembryonic endoderm cells. In other embodiments described herein, the cell population comprises human cells wherein at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85% or greater than at least about 85% of the human cells are human trophectoderm, ectoderm or extraembryonic endoderm cells. In some embodiments, the cell population includes non-human cells such as non-human feeder cells. In other embodiments, the cell population includes human feeder cells. In such embodiments, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or greater than at least about 95% of the human cells, other than said feeder cells, are human trophectoderm, ectoderm or extraembryonic endoderm cells.

In embodiments of the screening methods described herein, the cell population is contacted or otherwise provided with a candidate (test) differentiation factor. The candidate differentiation factor can comprise any molecule that may have the potential to promote the differentiation of human trophectoderm, ectoderm or extraembryonic endoderm cells. In some embodiments described herein, the candidate differentiation factor comprises a molecule that is known to be a differentiation factor for one or more types of cells. In alternate embodiments, the candidate differentiation factor comprises a molecule that in not known to promote cell differentiation. In preferred embodiments, the candidate differentiation factor comprises molecule that is not known to promote the differentiation of human trophectoderm, ectoderm or extraembryonic endoderm cells.

In some embodiments of the screening methods described herein, the candidate differentiation factor comprises a small molecule. In preferred embodiments, a small molecule is a molecule having a molecular mass of about 10,000 amu or less. In some embodiments, the small molecule comprises a retinoid. In some embodiments, the small molecule comprises retinoic acid.

In other embodiments described herein, the candidate differentiation factor comprises a polypeptide. The polypeptide can be any polypeptide including, but not limited to, a glycoprotein, a lipoprotein, an extracellular matrix protein, a cytokine, a chemokine, a peptide hormone, an interleukin or a growth factor. Preferred polypeptides include growth factors. In some preferred embodiments, the candidate differentiation factors comprise one or more growth factors selected from the group consisting of FGF10, FGF4, FGF2 and Wnt3B.

In some embodiments of the screening methods described herein, the candidate differentiation factors comprise one or more growth factors selected from the group consisting of Amphiregulin, B-lymphocyte stimulator, IL-16, Thymopoietin, TRAIL/Apo-2, Pre B cell colony enhancing factor, Endothelial differentiation-related factor 1 (EDF1), Endothelial monocyte activating polypeptide II, Macrophage migration inhibitory factor (MIF), Natural killer cell enhancing factor (NKEFA), Bone mophogenetic protein 2, Bone mophogenetic protein 8 (osteogeneic protein 2), Bone morphogenic protein 6, Bone morphogenic protein 7, Connective tissue growth factor (CTGF), CGI-149 protein (neuroendocrine differentiation factor), Cytokine A3 (macrophage inflammatory protein 1-alpha), Gliablastoma cell differentiation-related protein (GBDR1), Hepatoma-derived growth factor, Neuromedin U-25 precursor, Vascular endothelial growth factor (VEGF), Vascular endothelial growth factor B (VEGF-B), T-cell specific RANTES precursor, thymic dendritic cell-derived factor 1, Transferrin, Interleukin-1 (IL 1), Interleukin-2 (IL 2), Interleukin-3 (IL 3), Interleukin-4 (IL 4), Interleukin-5 (IL 5), Interleukin-6 (IL 6), Interleukin-7 (IL 7), Interleukin-8 (IL 8), Interleukin-9 (IL 9), Interleukin-10 (IL 10), Interleukin-11 (IL 11), Interleukin-12 (IL 12), Interleukin-13 (IL 13), Granulocyte-colony stimulating factor (G-CSF), Granulocyte macrophage colony stimulating factor (GM-CSF), Macrophage colony stimulating factor (M-CSF), Erythropoietin, Thrombopoietin, Vitamin D₃, Epidermal growth factor (EGF), Brain-derived neurotrophic factor, Leukemia inhibitory factor, Thyroid hormone, Basic fibroblast growth factor (bFGF), aFGF, FGF-4, FGF-6, Keratinocyte growth factor (KGF), Platelet-derived growth factor (PDGF), Platelet-derived growth factor-BB, beta nerve growth factor, activin A, Transforming growth factor beta 1 (TGF-β1), Interferon-α, Interferon-β, Interferon-γ, Tumor necrosis factor-α, Tumor necrosis factor-β, Burst promoting activity (BPA), Erythroid promoting activity (EPA), PGE₂, insulin growth factor-1 (IGF-I), IGF-II, Neutrophin growth factor (NGF), Neutrophin-3, Neutrophin 4/5, Ciliary neurotrophic factor, Glial-derived nexin, Dexamethasone, β-mercaptoethanol, Retinoic acid, Butylated hydroxyanisole, 5-azacytidine, Amphotericin B, Ascorbic acid, Ascrorbate, isobutylxanthine, indomethacin, β-glycerolphosphate, nicotinamide, DMSO, Thiazolidinediones, TWS119, oxytocin, vasopressin, melanocyte-stimulating hormone, corticortropin, lipotropin, thyrotropin, growth hormone, prolactin, luteinizing hormone, human chorionic gonadotropin, follicle stimulating hormone, corticotropin-releasing factor, gonadotropin-releasing factor, prolactin-releasing factor, prolactin-inhibiting factor, growth-hormone releasing factor, somatostatin, thyrotropin-releasing factor, calcitonin gene-related peptide, parathyroid hormone, glucagon-like peptide 1, glucose-dependent insulinotropic polypeptide, gastrin, secretin, cholecystokinin, motilin, vasoactive intestinal peptide, substance P, pancreatic polypeptide, peptide tyrosine tyrosine, neuropeptide tyrosine, insulin, glucagon, placental lactogen, relaxin, angiotensin II, calctriol, atrial natriuretic peptide, and melatonin, thyroxine, triiodothyronine, calcitonin, estradiol, estrone, progesterone, testosterone, cortisol, corticosterone, aldosterone, epinephrine, norepinepherine, androstiene, calcitriol, collagen, Dexamethasone, β-mercaptoethanol, Retinoic acid, Butylated hydroxyanisole, 5-azacytidine, Amphotericin B, Ascorbic acid, Ascrorbate, isobutylxanthine, indomethacin, β-glycerolphosphate, nicotinamide, DMSO, Thiazolidinediones, and TWSI119.

In some embodiments of the screening methods described herein, the candidate differentiation factor is provided to the cell population in one or more concentrations. In some embodiments, the candidate differentiation factor is provided to the cell population so that the concentration of the candidate differentiation factor in the medium surrounding the cells ranges from about 0.1 ng/ml to about 10 μg/ml. In some embodiments, the concentration of the candidate differentiation factor in the medium surrounding the cells ranges from about 1 ng/ml to about 1 μg/ml. In other embodiments, the concentration of the candidate differentiation factor in the medium surrounding the cells ranges from about 10 ng/ml to about 100 μg/ml. In still other embodiments, the concentration of the candidate differentiation factor in the medium surrounding the cells ranges from about 100 ng/ml to about 10 μg/ml. In preferred embodiments, the concentration of the candidate differentiation factor in the medium surrounding the cells is about 5 ng/ml, about 25 ng/ml, about 50 ng/ml, about 75 ng/ml, about 100 ng/ml, about 125 ng/ml, about 150 ng/ml, about 175 ng/ml, about 200 ng/ml, about 225 ng/ml, about 250 ng/ml, about 275 ng/ml, about 300 ng/ml, about 325 ng/ml, about 350 ng/ml, about 375 ng/ml, about 400 ng/ml, about 425 ng/ml, about 450 ng/ml, about 475 ng/ml, about 500 ng/ml, about 525 ng/ml, about 550 ng/ml, about 575 ng/ml, about 600 ng/ml, about 625 ng/ml, about 650 ng/ml, about 675 ng/ml, about 700 ng/ml, about 725 ng/ml, about 750 ng/ml, about 775 ng/ml, about 800 ng/ml, about 825 ng/ml, about 850 ng/ml, about 875 ng/ml, about 900 ng/ml, about 925 ng/ml, about 950 ng/ml, about 975 ng/ml, about 1 μg/ml, about 2 μg/ml, about 3 μg/ml, about 4 μg/ml, about 5 μg/ml, about 6 μg/ml, about 7 μg/ml, about 8 μg/ml, about 9 μg/ml, about 10 μg/ml, about 11 μg/ml, about 12 μg/ml, about 13 μg/ml, about 14 μg/ml, about 15 μg/ml, about 16 μg/ml, about 17 μg/ml, about 18 μg/ml, about 19 μg/ml, about 20 μg/ml, about 25 μg/ml, about 50 μg/ml, about 75 μg/ml, about 100 μg/ml, about 125 μg/ml, about 150 μg/ml, about 175 μg/ml, about 200 μg/ml, about 250 μg/ml, about 300 μg/ml, about 350 μg/ml, about 400 μg/ml, about 450 μg/ml, about 500 μg/ml, about 550 μg/ml, about 600 μg/ml, about 650 μg/ml, about 700 μg/ml, about 750 μg/ml, about 800 μg/ml, about 850 μg/ml, about 900 μg/ml, about 950 μg/ml, about 1000 μg/ml or greater than about 1000 μg/ml.

In certain embodiments of the screening methods described herein, the cell population is provided with a candidate differentiation factor which comprises any molecule other than an FGF family growth factor, BMP, SU5402, follistatin, noggin, a growth factor of the TGFβ superfamily and/or a retinoid.

It will be appreciated that any of the steps of the screening methods described herein can take place under conditions where PI-3-kinase signaling is limited. Alternatively, any of the steps of the screening methods described herein can take place under conditions where PI-3-kinsase is not limited.

It will be appreciated that PI-3-kinase signaling may be limited in culture by any of the methods described herein, including, for example, cell culture conditions where levels of serum, insulin, insulin analogs, insulin-like growth factors, insulin-like growth factor analogs or insulin-mimetic compounds are provided in a maintenance, growth or differentiation medium at less than a substantial concentration or effective amount. As an alternative, PI-3-kinase signaling may be limited in culture by utilizing a maintenance, growth or differentiation medium that lacks serum, insulin, insulin analogs, insulin-like growth factors, insulin-like growth factor analogs and/or insulin-mimetic compounds. As another alternative, PI-3-kinase signaling may be limited by adding one or more inhibitors of PI-3-kinase to the cell culture medium. As previously described, examples of PI-3-kinase pathway inhibitors include P-13-kinase antagonists, antagonists of the PI-3-kinase signal transduction cascade, compounds that decrease the synthesis or expression of endogenous PI-3-kinase, compounds that decrease release of endogenous PI-3-kinase, and compounds that inhibit activators of PI-3-kinase activity.

In some embodiments, steps of the screening methods described herein comprise determining expression of at least one marker at a first time point and a second time point. In some of these embodiments, the first time point can be prior to or at approximately the same time as providing the cell population with the candidate differentiation factor. Alternatively, in some embodiments, the first time point is subsequent to providing the cell population with the candidate differentiation factor. In some embodiments, expression of a plurality of markers is determined at a first time point.

In addition to determining expression of at least one marker at a first time point, some embodiments of the screening methods described herein contemplate determining expression of at least one marker at a second time point, which is subsequent to the first time point and which is subsequent to providing the cell population with the candidate differentiation factor. In such embodiments, expression of the same marker is determined at both the first and second time points. In some embodiments, expression of a plurality of markers is determined at both the first and second time points. In such embodiments, expression of the same plurality of markers is determined at both the first and second time points. In some embodiments, marker expression is determined at a plurality of time points, each of which is subsequent to the first time point, and each of which is subsequent to providing the cell population with the candidate differentiation factor. In certain embodiments, marker expression is determined by Q-PCR. In other embodiments, marker expression is determined by immunocytochemistry.

In certain embodiments of the screening methods described herein, the marker having its expression is determined at the first and second time points is a marker that is associated with the differentiation of human trophectoderm to cells which are the precursors of cells which make up the mural or polar trophoblast. In some embodiments, the cells of the mural or polar trophoblast comprise terminally differentiated cells. In other embodiments of the screening methods described herein, the marker having its expression is determined at the first and second time points is a marker that is associated with the differentiation of human ectoderm to cells which are the precursors of cells which make up neural or non-neural ectoderm. In some embodiments, the cells of the neural or non-neural ectoderm comprise terminally differentiated cells. In certain embodiments of the screening methods described herein, the marker having its expression is determined at the first and second time points is a marker that is associated with the differentiation of human extraembryonic endoderm to cells which are the precursors of cells which make up the visceral endoderm or parietal endoderm. In some embodiments, the cells of the visceral endoderm or parietal endoderm comprise terminally differentiated cells.

In some embodiments of the screening methods described herein, sufficient time is allowed to pass between providing the cell population with the candidate differentiation factor and determining marker expression at the second time point. Sufficient time between providing the cell population with the candidate differentiation factor and determining expression of the marker at the second time point can be as little as from about 1 hour to as much as about 10 days. In some embodiments, the expression of at least one marker is determined multiple times subsequent to providing the cell population with the candidate differentiation factor. In some embodiments, sufficient time is at least about 1 hour, at least about 6 hours, at least about 12 hours, at least about 18 hours, at least about 24 hours, at least about 30 hours, at least about 36 hours, at least about 42 hours, at least about 48 hours, at least about 54 hours, at least about 60 hours, at least about 66 hours, at least about 72 hours, at least about 78 hours, at least about 84 hours, at least about 90 hours, at least about 96 hours, at least about 102 hours, at least about 108 hours, at least about 114 hours, at least about 120 hours, at least about 126 hours, at least about 132 hours, at least about 138 hours, at least about 144 hours, at least about 150 hours, at least about 156 hours, at least about 162 hours, at least about 168 hours, at least about 174 hours, at least about 180 hours, at least about 186 hours, at least about 192 hours, at least about 198 hours, at least about 204 hours, at least about 210 hours, at least about 216 hours, at least about 222 hours, at least about 228 hours, at least about 234 hours or at least about 240 hours.

In some embodiments of the methods described herein, it is further determined whether the expression of the marker at the second time point has increased or decreased as compared to the expression of this marker at the first time point. An increase or decrease in the expression of the at least one marker indicates that the candidate differentiation factor is capable of promoting the differentiation of the trophectoderm, ectoderm or extraembryonic endoderm cells. Similarly, if expression of a plurality of markers is determined, it is further determined whether the expression of the plurality of markers at the second time point has increased or decreased as compared to the expression of this plurality of markers at the first time point. An increase or decrease in marker expression can be determined by measuring or otherwise evaluating the amount, level or activity of the marker in the cell population at the first and second time points. Such determination can be relative to other markers, for example housekeeping gene expression, or absolute. In certain embodiments, wherein marker expression is increased at the second time point as compared with the first time point, the amount of increase is at least about 2-fold, at least about 5-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold or more than at least about 100-fold. In some embodiments, the amount of increase is less than 2-fold. In embodiments where marker expression is decreased at the second time point as compared with the first time point, the amount of decrease is at least about 2-fold, at least about 5-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold or more than at least about 100-fold. In some embodiments, the amount of decrease is less than 2-fold.

### Production of Mesendoderm and Definitive Endoderm Under Conditions That Decrease or Limit PI-3-K Signaling

Methods for differentiating human pluripotent cell cultures to form mesendoderm and/or definitive endoderm cells are known. For example, co-pending and co-owned U.S. patent application Ser. No. 11/021,618, entitled DEFINITIVE ENDODERM, filed Dec. 23, 2004, the disclosure of which is incorporated herein by reference in its entirety, describes methods of preparing in vitro human definitive endoderm cell cultures and/or purified cell populations from human pluripotent cells, such as human embryonic stem cells. Additionally, methods of monitoring the production of definitive endoderm cell cultures and/or cell populations is described in detail. Methods of preparing in vitro human pre-primitive streak and mesendoderm cell cultures and/or purified cell populations from human pluripotent cells, such as human embryonic stem cells, is described in co-pending and co-owned U.S. patent application Ser. No. 11/474,211, entitled PREPRIMITIVE STREAK AND MESENDODERM CELLS, filed Jun. 23, 2006, the disclosure of which is incorporated herein by reference in its entirety. Methods of monitoring the production of the pre-primitive streak and mesendoderm cell cultures and/or cell populations is also described in detail.

It will be appreciated that the methods described herein for decreasing or limiting phosphatidylinositol-3-kinase (PI-3-K) pathway signaling in the production of ectoderm, trophectoderm and extraembryonic endoderm cells can be also used in the production of mesendoderm and definitive endoderm cells from human pluripotent cells without undue experimentation. In some embodiments, one or more inhibitors of PI-3-K signaling pathway can be provided to a human pluripotent cell culture, such as a human embryonic stem cell culture, under conditions that promote differentiation of the cells to mesendoderm and/or definitive endoderm cells as described in U.S. patent application Ser. No. 11/021,618 and U.S. patent application Ser. No. 11/474,211. The production of mesendoderm cells and/or definitive endoderm cells in such cell cultures can be monitored using the methods described in the above-referenced patent applications. In other embodiments, a human pluripotent cell culture, such as a human embryonic stem cell culture, is differentiated to mesendoderm and/or definitive endoderm cells as described in U.S. patent application Ser. No. 11/021,618 and U.S. patent application Ser. No. 11/474,211 under conditions described herein, which decrease or limit the PI-3-K pathway signaling. The production of mesendoderm cells and/or definitive endoderm cells in such cell cultures can be monitored using the methods described in the above-referenced patent applications. Decreasing or limiting phosphatidylinositol-3-kinase (PI-3-K) pathway signaling during the differentiation of human pluripotent cells to human mesendoderm cells and/or human definitive endoderm cells was shown to improve the efficiency of the production of human mesendoderm cells and/or human definitive endoderm cells and to greatly increase the overall number and concentration of human mesendoderm cells and/or human definitive endoderm cells produced from the human pluripotent cell culture.

### EXAMPLES

Many of the examples below describe the use of pluripotent human cells. Methods of producing pluripotent human cells are well known in the art and have been described numerous scientific publications, including U.S. Pat. Nos. 5,453,357, 5,670,372, 5,690,926, 6,090,622, 6,200,806 and 6,251,671 as well as U.S. Patent Application Publication No. 2004/0229350, the disclosures of which are incorporated herein by reference in their entireties.

### Example 1

### Human ES cells

For our studies, we employed human embryonic stem cells, which are pluripotent and can divide seemingly indefinitely in culture while maintaining a normal karyotype. ES cells were derived from the 5-day-old embryo inner cell mass using either immunological or mechanical methods for isolation. In particular, the human embryonic stem cell line hESCyt-25 was derived from a supernumerary frozen embryo from an in vitro fertilization cycle following informed consent by the patient. Upon thawing the hatched blastocyst was plated on mouse embryonic fibroblasts (MEF), in ES medium ((DMEM, 20% FBS, non essential amino acids, beta-mercaptoethanol, and FGF2). The embryo adhered to the culture dish and after approximately two weeks, regions of undifferentiated hESCs were transferred to new dishes with MEFs. Transfer was accomplished with mechanical cutting and a brief digestion with dispase, followed by mechanical removal of the cell clusters, washing and re-plating. Since derivation, hESCyt-25 has been serially passaged over 100 times. We employed the hESCyt-25 human embryonic stem cell line as our starting material. Additionally, we have used other hESC lines developed both by us and by others including, but not limited to, Cyt-49, Cyt-203, BG01, BG02 and BG03.

It will be appreciated by those of skill in the art that stem cells or other pluripotent cells can also be used as starting material for the differentiation procedures described herein. For example, cells obtained from embryonic gonadal ridges, which can be isolated by methods known in the art, can be used as pluripotent cellular starting material.

### Example 2

### hESCyt-25 Characterization

The human embryonic stem cell line, hESCyt-25 has maintained a normal morphology, karyotype, growth and self-renewal properties over 18 months in culture. This cell line displays strong immunoreactivity for the OCT4, SSEA-4 and TRA-1-60 antigens, all of which are characteristic of undifferentiated hESCs and displays alkaline phosphatase activity as well as a morphology identical to other established hESC lines. Furthermore, the human stem cell line, hESCyt-25, also readily forms embryoid bodies (EBs) when cultured in suspension. As a demonstration of its pluripotent nature, hESCyT-25 differentiates into various cell types that represent the three principal germ layers. Ectoderm production was demonstrated by Q-PCR for ZIC1 as well as immunocytochemistry (ICC) for nestin and more mature neuronal markers. Immunocytochemical staining for β-III tubulin was observed in clusters of elongated cells, characteristic of early neurons. Cells differentiated in monolayer expressed AFP in sporadic patches as demonstrated by immunocytochemical staining. The hESCyT-25 cell line was also capable of forming definitive endoderm, as validated by real-time quantitative polymerase chain reaction (Q-PCR) and immunocytochemistry for SOX17, in the absence of AFP expression. To demonstrate differentiation to mesoderm, differentiating EBs were analyzed for Brachyury gene expression at several time points. Brachyury expression increased progressively over the course of the experiment. In view of the foregoing, the hESCyT-25 line is pluripotent as shown by the ability to form cells representing the three germ layers.

TABLE 2

| Germ Layer | Gene | Expression Domains |
|---|---|---|
| Endoderm | SOX17 | definitive, visceral and parietal endoderm |
| | MIXL1 | endoderm and mesoderm |
| | GATA4 | definitive and primitive endoderm |
| | HNF3b | definitive endoderm and primitive endoderm, |
| | | mesoderm, neural plate |
| | GSC | endoderm and mesoderm |
| Extra- | SOX7 | visceral endoderm |
| embryonic | AFP | visceral endoderm, liver |
| | SPARC | parietal endoderm |
| | TM | parietal endoderm/trophectoderm |
| Ectoderm | ZIC1 | neural tube, neural progenitors |
| Mesoderm | BRACH | nascent mesoderm |

### Example 3

### Control of hESCs Lineage Specification to Four Initial Cell Fates Using Three Different Signaling Environments

Human ESCs (CyT203) were grown in growth media on a mouse fibroblast feeder layer. On the day differentiation began, cultures were washed once with phosphate buffered saline (PBS) and then placed into differentiation media. Differentiation media consisted of RPMI with penicillin/streptomycin and Glutamax containing 0% FBS on the first day, 0.2% v/v FBS on the second day and 2% v/v FBS on days 3-5. Growth factors were used at the following concentrations: activin A at 100 ng/ml, follistatin at 100 ng/ml, noggin at 200 ng/ml, BMP4 at 100 ng/ml, and SU5402 at 5 μM. Individual cultures received the same growth factor cocktail with or without the addition of 10 μg/ml human recombinant insulin for all 5 days of differentiation as indicated. Samples were taken at daily intervals for analysis of gene expression using QPCR to monitor the differentiation of the populations in response to the different growth factor treatments. At the conclusion of the treatment phase the cells were fixed in 4% paraformaldehyde for 15 min at room temperature and reacted with antibodies to SOX17 or PAX6.

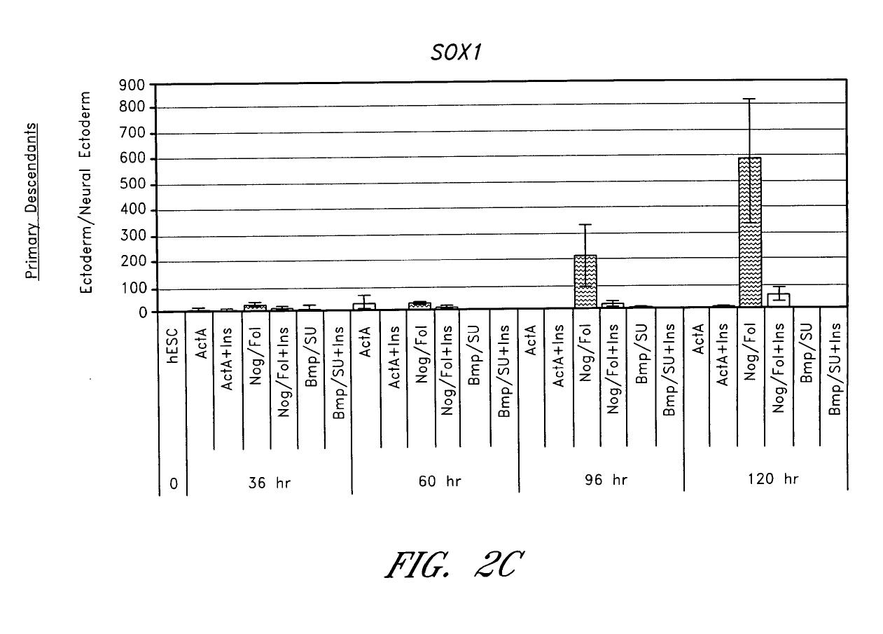

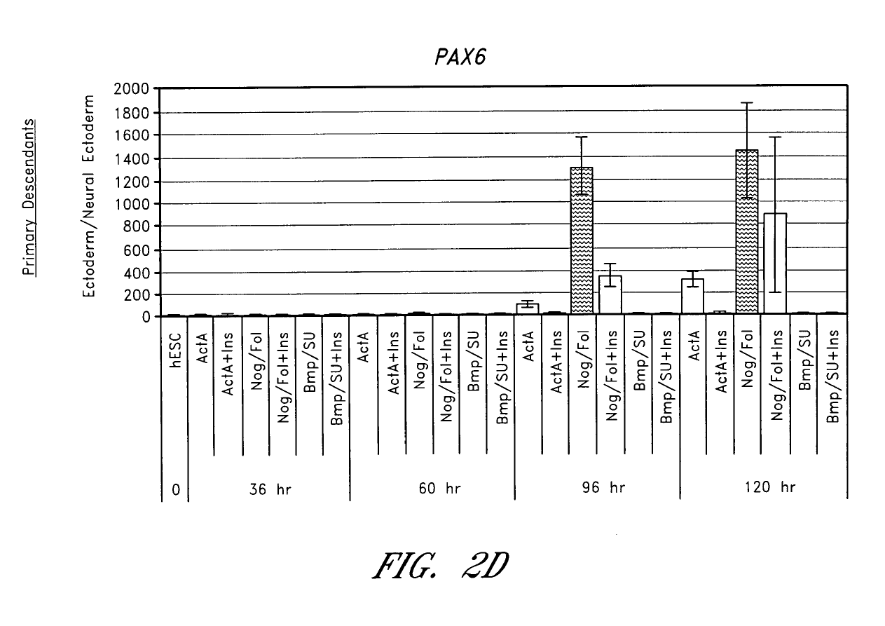

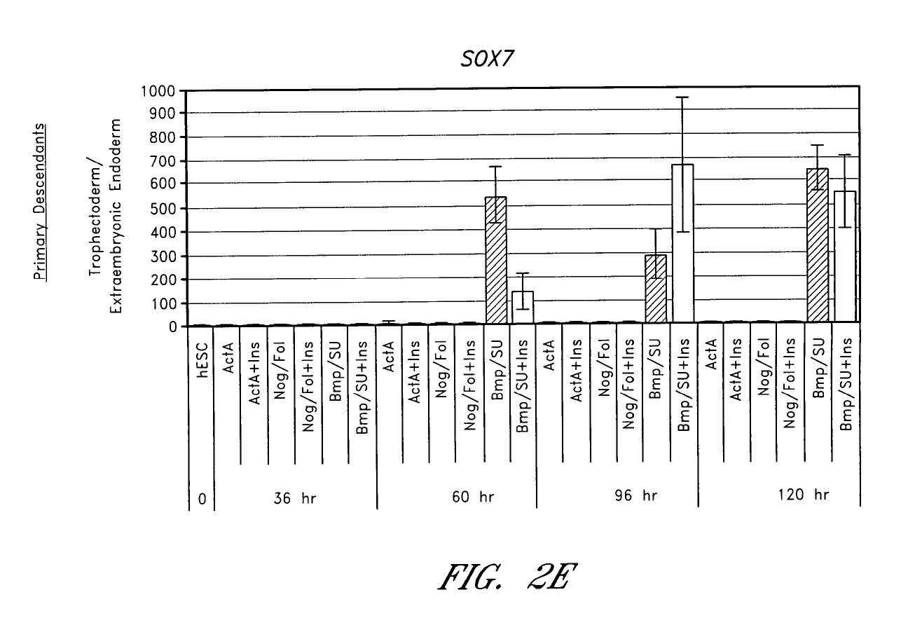

Three different growth factor conditions were found that efficiently induce differentiation of hESCs to the four initial fates of ESC differentiation in the cultures lacking insulin. The effects of these three growth factor conditions were demonstrated through specific patterns of gene expression of markers for these early lineages, as shown in FIG. 2A-H. The differentiation of hESCs using activin A induced initial fate commitment to mesendoderm as indicated by elevated expression of brachyury and WNT3A at 36 and 48 hours (FIGS. 2A and B). In the continued presence of activin A, the mesendoderm was further differentiated to definitive endoderm as indicated by elevated expression of SOX17 and GSC (FIGS. 2G and H). Treatment of hESCs with noggin and follistatin induced the differentiation to ectoderm and neural ectoderm as indicated by the expression of SOX1 and PAX6 (FIGS. 2C and D). The induction of PAX6 mRNA in the ActA treatment was likely a result of the formation of anterior definitive endoderm which has been shown in mouse embryos to be a robust inducer of early neurectoderm differentiation. Treatment of hESCs with BMP4 and the FGFR inhibitor SU5402 induced the differentiation to extraembryonic endoderm and trophectoderm as indicated by the expression of SOX7 and CDX2, respectively (FIGS. 2E and F). As can be seen from FIGS. 2A-H, the addition of insulin to the medium significantly reduced the production of cells of each of the primary lineages.

### Example 4

### Reduction in PI-3-K Signaling Creates a Permissive State for the Differentiation of hESCs

Human embryonic stem cells (CyT203) were grown in growth media on a mouse fibroblast feeder layer. On the day differentiation began, cultures were washed once with phosphate buffered saline (PBS) and then placed into differentiation media. Differentiation media consisted of RPMI with penicillin/streptomycin and Glutamax containing 0% FBS on the first day, 0.2% v/v FBS on the second day and 2% v/v FBS on days 3-5. Cultures were treated with SU5402 at 5 μM with or without the addition of human recombinant insulin at 10 μg/ml. Individual cultures received the same growth factor cocktail for all 5 days of differentiation as indicated. Samples were taken at daily intervals for analysis of gene expression using QPCR to monitor the differentiation of the populations in response to the different growth factor treatments. At the conclusion of the treatment phase the cells were fixed in 4% paraformaldehyde for 15 min at room temperature and reacted with antibodies to SOX17, OCT4 or PAX6.

It was observed that the presence of high levels of insulin in the media, which signals through the PI-3-kinase signal transduction pathway, resulted in a decrease in the efficiency with which the hESCs differentiated to each of the 4 primary lineages as indicated by the decreased expression of brachyury, WNT3A, PAX6, SOX1, SOX7, and CDX2 as shown in FIGS. 2A-H. This indicated that elevated signaling through the PI-3-kinase pathway created resistance to effective differentiation of hESCs and that removal of PI-3-kinase signaling activity, achieved in this case via the removal of insulin from the media, established a permissive state for hESC differentiation. Thus, in the presence of reduced insulin (reduced PI-3-kinase signaling) and when the hESCs were provided differentiation signals, they responded rapidly and synchronously to differentiate in a lineage-specific manner.

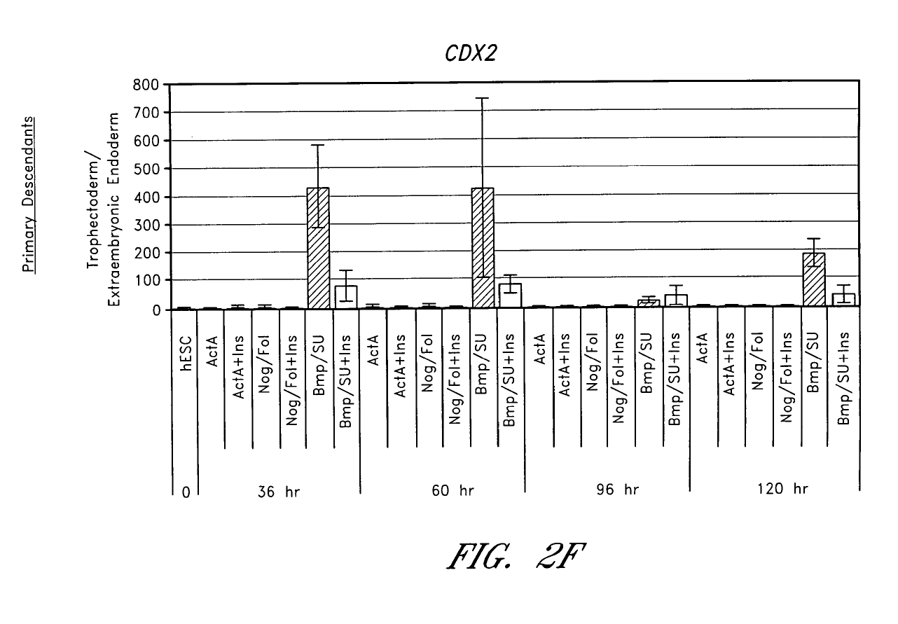

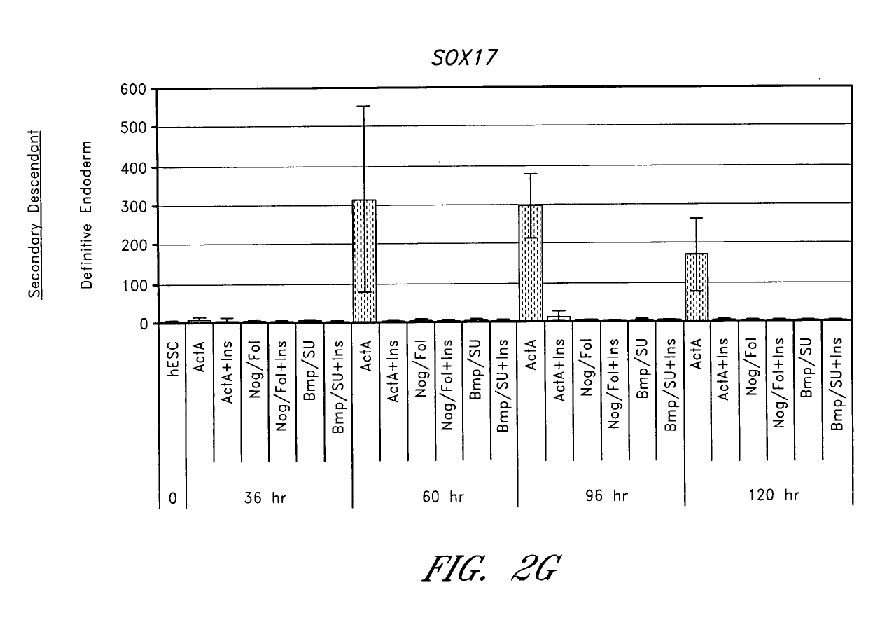

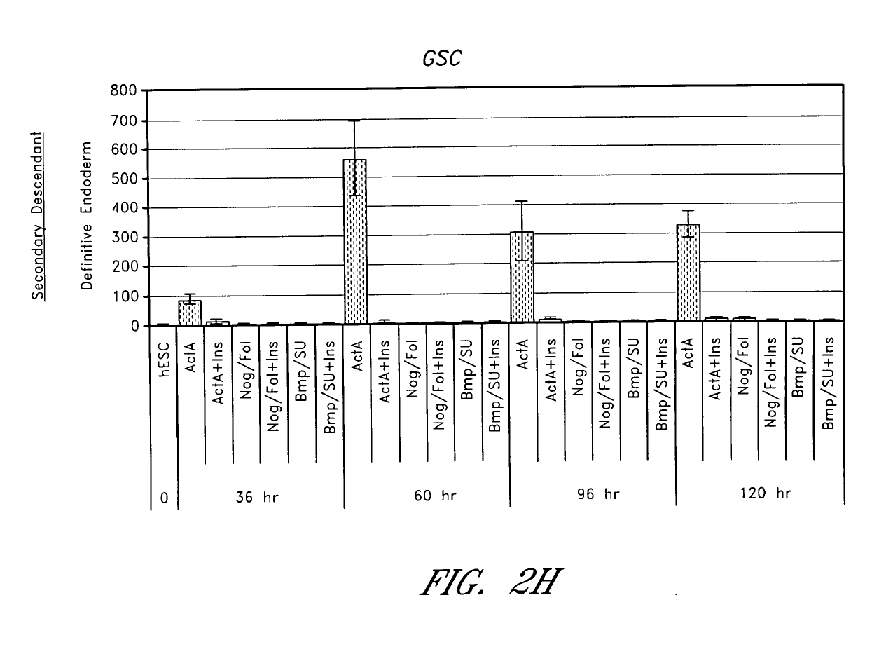

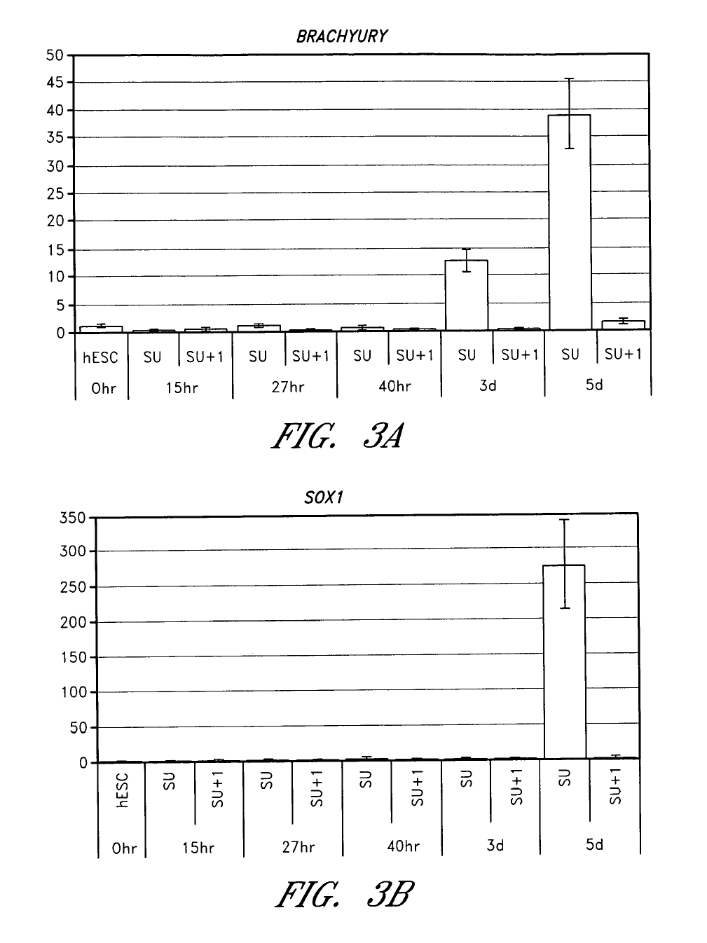

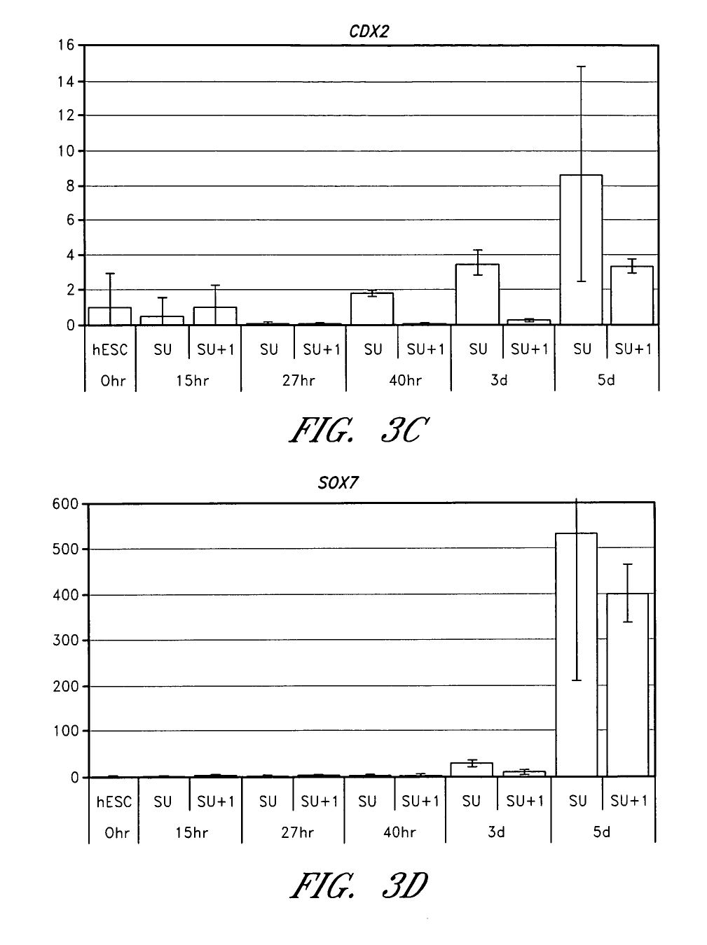

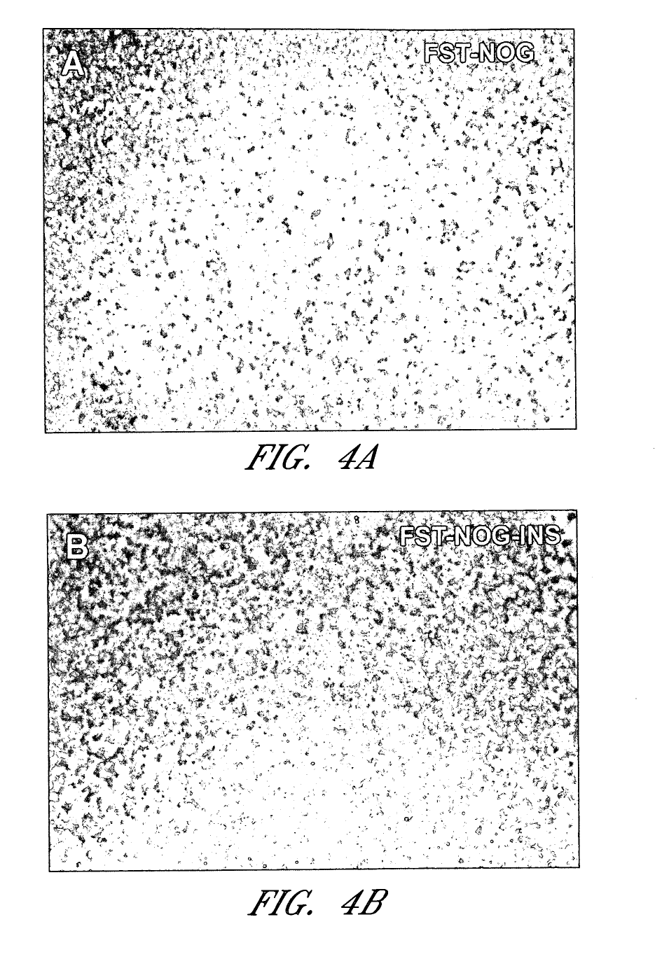

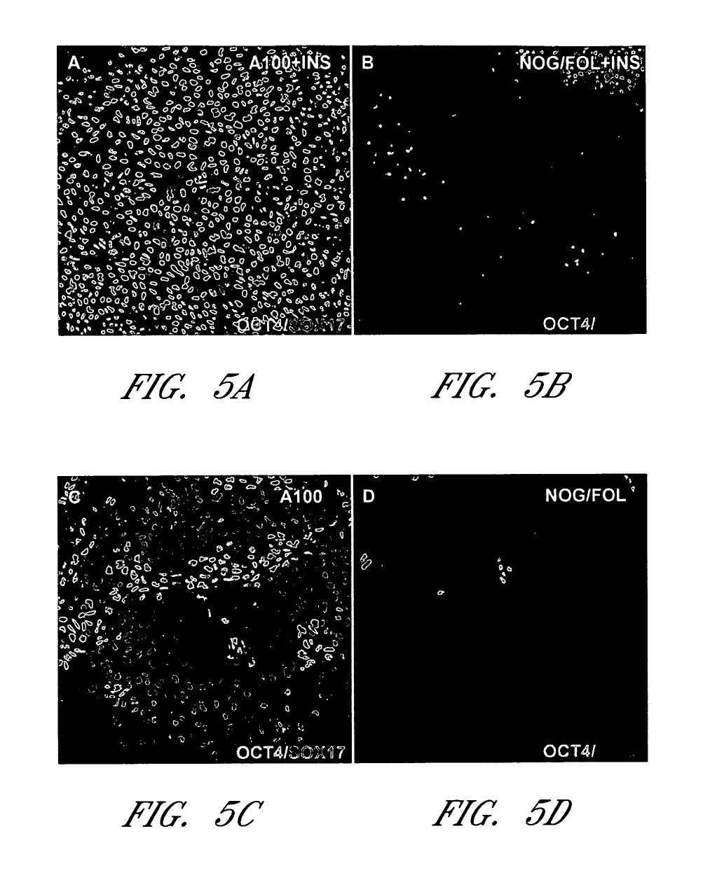

Additionally, it was found that treatment of hESCs with the FGFR inhibitor SU5402 provided a generalized signaling environment whereby the ESCs made all available fate choices as shown by elevated expression of brachyury, SOX1, SOX7 and CDX2. This generalized differentiation was also inhibited strongly by the presence of insulin as demonstrated in FIGS. 3A-D.

In addition to mRNA expression data, it was observed that the presence of insulin in the media reduced the number of PAX6-positive cells in response to noggin/follistatin treatment as demonstrated by immunofluorescence (FIGS. 4A-B). Also, it was observed by immunofluorescence that when high levels of insulin were present, there was maintenance of OCT4-positive cell numbers (FIGS. 5A,B) and a decrease in the number of SOX17-positive cells in response to activin A treatment (FIGS. 5A,C) or decrease in PAX6-positive cells in response to noggin/follistatin treatment (FIGS. 5B,D), as demonstrated by immunofluorescence.

### REFERENCES

Numerous literature and patent references have been cited in the present patent application. Each and every reference that cited in this patent application is incorporated by reference herein in its entirety.

For some references, the complete citation is in the body of the text. For other references the citation in the body of the text is by author and year, the complete citation being as follows:
Alexander, J., Rothenberg, M., Henry, G. L., and Stainier, D. Y. (1999). Casanova plays an early and essential role in endoderm formation in zebrafish. Dev Biol 215, 343-357.
Alexander, J., and Stainier, D. Y. (1999). A molecular pathway leading to endoderm formation in zebrafish. Curr Biol 9, 1147-1157.
Bongso, A., Fong, C. Y., Ng, S. C., and Ratnam, S. (1994). Isolation and culture of inner cell mass cells from human blastocysts. Hum Reprod 9, 2110-2117.
Cocco, M. T., Onnis, V., Ponticelli, G., Meier, B., Rehder, D., Garribba, E., Micera, G., (2006). Synthesis, characterization and insulin-mimetic activity of oxovanadium(IV) complexes with amidrazone derivatives. J. Inorg. Biochem.
Conlon, F. L., Lyons, K. M., Takaesu, N., Barth, K. S., Kispert, A., Herrmann, B., and Robertson, E. J. (1994). A primary requirement for nodal in the formation and maintenance of the primitive streak in the mouse. Development 120, 1919-1928.
Dougan, S. T., Warga, R. M., Kane, D. A., Schier, A. F., and Talbot, W. S. (2003). The role of the zebrafish nodal-related genes squint and cyclops in patterning of mesendoderm. Development 130, 1837-1851.
Feldman, B., Gates, M. A., Egan, E. S., Dougan, S. T., Rennebeck, G., Sirotkin, H. I., Schier, A. F., and Talbot, W. S. (1998). Zebrafish organizer development and germ-layer formation require nodal-related signals. Nature 395, 181-185.
Hogan, B. L. (1997). Pluripotent embryonic cells and methods of making same (U.S.A., Vanderbilt University).
Hudson, C., Clements, D., Friday, R. V., Stott, D., and Woodland, H. R. (1997). Xsox17alpha and -beta mediate endoderm formation in Xenopus. Cell 91, 397-405.
Kanai-Azuma, M., Kanai, Y., Gad, J. M., Tajima, Y., Taya, C., Kurohmaru, M., Sanai, Y., Yonekawa, H., Yazaki, K., Tam, P. P., and Hayashi, Y. (2002). Depletion of definitive gut endoderm in Sox17-null mutant mice. Development 129, 2367-2379.
Kikuchi, Y., Agathon, A., Alexander, J., Thisse, C., Waldron, S., Yelon, D., Thisse, B., and Stainier, D. Y. (2001). casanova encodes a novel Sox-related protein necessary and sufficient for early endoderm formation in zebrafish. Genes Dev 15, 1493-1505.
Kimelman, D., and Griffin, K. J. (2000). Vertebrate mesendoderm induction and patterning. Curr Opin Genet Dev 10, 350-356.
Mehdi M. Z., Vardatsikos, G., Pandey, S. K., Srivastava, A. K. (2006). Invovlement of insulin-like growth factor type 1 receptor and protein kinase C delta in Bis(maltolato)oxovanadium(IV)-induced phosphorylation of protein kinase B in HepG2 cells. Biochemistry 45, 11605-11615.
Reubinoff, B. E., Pera, M. F., Fong, C. Y., Trounson, A., and Bongso, A. (2000). Embryonic stem cell lines from human blastocysts: somatic differentiation in vitro. Nat Biotechnol 18, 399-404.
Sakurai H. and Adachi, Y. (2005). The pharmacology of the insulinomimetic effect of zinc complexes. BioMetals 18, 319-323.
Schoenwolf, G. C., and Smith, J. L. (2000). Gastrulation and early mesodermal patterning in vertebrates. Methods Mol Biol 135, 113-125.
Shamblott, M. J., Axelman, J., Wang, S., Bugg, E. M., Littlefield, J. W., Donovan, P. J., Blumenthal, P. D., Huggins, G. R., and Gearhart, J. D. (1998). Derivation of pluripotent stem cells from cultured human primordial germ cells. Proc Natl Acad Sci USA 95, 13726-13731.
Thomson, J. A., ltskovitz-Eldor, J., Shapiro, S. S., Waknitz, M. A., Swiergiel, J. J., Marshall, V. S., and Jones, J. M. (1998). Embryonic stem cell lines derived from human blastocysts. Science 282, 1145-1147.
Tremblay, K. D., Hoodless, P. A., Bikoff, E. K., and Robertson, E. J. (2000). Formation of the definitive endoderm in mouse is a Smad2-dependent process. Development 127, 3079-3090.
Vincent, S. D., Dunn, N. R., Hayashi, S., Norris, D. P., and Robertson, E. J. (2003). Cell fate decisions within the mouse organizer are governed by graded Nodal signals. Genes Dev 17, 1646-1662.
Wells, J. M., and Melton, D. A. (1999). Vertebrate endoderm development. Annu Rev Cell Dev Biol 15, 393-410.
Wells, J. M., and Melton, D. A. (2000). Early mouse endoderm is patterned by soluble factors from adjacent germ layers. Development 127, 1563-1572.
Zhou, X., Sasaki, H., Lowe, L., Hogan, B. L., and Kuehn, M. R. (1993). Nodal is a novel TGF-beta-like gene expressed in the mouse node during gastrulation. Nature 361, 543-547.

1.-125. (canceled)

126. A method of differentiating human pluripotent cells, said method comprising the steps of
obtaining a population of human pluripotent cells, providing said population of human pluripotent cells with at least one differentiation factor in an amount sufficient to promote differentiation of said human pluripotent cells to cells of a cell lineage selected from the group consisting of mesendoderm and definitive endoderm,
contacting said population of human pluripotent cells with a culture medium that decreases or limits phosphotidylinositol-3-kinase (PI-3-kinase) signaling, and
incubating said population of human pluripotent cells in said culture medium for a sufficient time to allow said pluripotent cells to differentiate into cells of a cell lineage selected from the group consisting of mesendoderm and definitive endoderm.

127. The method of claim 126, wherein the differentiation factor is selected from the group consisting of Activin A, Activin B, nodal and a combination thereof.

128. The method of claim 126, wherein the differentiation factor is Activin A.

129. The method of claim 126, wherein the differentiation factor is nodal.

130. The method of claim 126, further comprising providing said human pluripotent cell with a fibroblast growth factor receptor (FGFR) inhibitor.

131. The method of claim 130, wherein the FGFR inhibitor is SU5402.

132. The method of claim 126, wherein sufficient time to allow the pluripotent cells to differentiate into cells of a cell lineage is determined by detecting the presence of the cell lineage in the population of human pluripotent cells, wherein detecting the presence of the cell lineage in the cell population comprises detecting the expression of at least one marker selected from the group consisting of Brachyury, Wnt3, SOX17 and GSC.

133. The method of claim 126, wherein detecting the presence of mesendoderm cells in the population of human pluripotent cells comprises detecting the expression of at least Brachyury or Wnt3.

134. The method of claim 126, wherein detecting the presence of definitive endoderm in the population of human pluripotent cells comprises detecting the expression of at least SOX17 and GSC.

135. The method of claim 126, wherein the expression of at least one of said markers is determined by Q-PCR.

136. The method of claim 126, wherein the expression of at least one of said markers is determined by immunocytochemistry.

137. The method of claim 126, wherein said pluripotent cells are human embryonic stem cells.

138. The method of claim 136, wherein said human embryonic stem cell are derived from a tissue selected from either the morula or the inner cell mass (ICM) of an embryo.

139. The method of claim 136, wherein said human embryonic stem cells are derived from a preimplantation embryo.

140. The method of claim 126, wherein said PI-3-K pathway inhibitor is selected from the group consisting of rapamycin, LY 294002, wortmannin, lithium chloride, Akt inhibitor I, Akt inhibitor II (SH-5), Akt inhibitor III (SH-6), NL-71-101 and combinations thereof.

141. The method of claim 126 wherein said PI-3-K pathway inhibitor is LY 294002.

142. The method of claim 126, wherein said culture medium lacks a substantial concentration or an effective amount of a PI-3-K pathway activator.

143. The method of claim 126, wherein said culture medium lacks a substantial concentration or an effective amount of a PI-3-K pathway activator selected from the group consisting of serum, insulin, insulin analogs, insulin-like growth factors, insulin-like growth factor analogs, insulin mimetics, and combinations thereof.

144. The method of claim 126, wherein said culture medium lacks a substantial concentration or an effective amount of a PI-3-K pathway activator selected from the group consisting of insulin, insulin-like growth factor, and combinations thereof.

145. The method of claim 126, wherein said population of human pluripotent cells is an adherent cell culture.
